(19) United States
(12) Patent Application Publication
(10) Pub. No.: US 2011/0003808 A1
(43) Pub. Date: Jan. 6, 2011

(54) OCTAHYDROQUINOLIZINES FOR ANTIDIABETIC TREATMENT

(75) Inventors: Immanuel Adorjan, Tulln (AT); Leonhardt Bauer, Wien (AT); Klaus Frobel, Wuppertal (DE); Clemens Fuernsinn, Wien (AT)

Correspondence Address: WORKMAN NYDEGGER;1000 Eagle Gate Tower, 60 East South Temple, Salt Lake City, UT, 84111, US

(73) Assignee: 55PHARMA DRUG DISCOVERY & DEVELOPMENT AG, Wien (AT)

(21) Appl. No.: 12/809,513

(22) PCT Filed: Dec. 17, 2008
(86) PCT No.: PCT/AT08/00458

§ 371 Date: Aug. 4, 2010

(30) Foreign Application Priority Data
Dec. 19, 2007 (EP) 07450235.2
Aug. 25, 2008 (AT) A 1315/2008

(51) Int. Cl. A61K 31/5377; C07D 455/02; A61K 31/4375; C07D 405/14; A61K 31/438; C07D 413/14; C07D 401/14; A61K 31/496; A61P 3/10; A61P 9/00; A61P 3/00; A61P 3/04

(52) U.S. Cl. 514/233.2; 546/138; 514/306; 546/18; 514/278; 544/127; 544/362; 514/253.04

(57) ABSTRACT

This invention relates to novel octahydroquinolizines for treatment or prevention of diabetes mellitus and its complications, for treatment or prevention of hyperlipidemia, for treatment of diabetic dyslipidemia, for treatment or prevention of the metabolic syndrome, for treatment of diseases related to metabolic dysfunction, for treatment of obesity or obesity-related diseases. The invention also includes pharmaceutical compositions and kits comprising these compounds alone or in combination with other drugs or compounds aiming towards an improved treatment or prevention of the aforementioned diseases or syndromes in humans or animals.

### FIELD OF THE INVENTION

This invention relates to novel octahydroquinolizines for treatment or prevention of diabetes mellitus and its complications, for treatment or prevention of hyperlipidemia, for treatment of diabetic dyslipidemia, for treatment or prevention of the metabolic syndrome, for treatment of diseases related to metabolic dysfunction, for treatment of obesity or obesity-related diseases. The invention also includes pharmaceutical compositions and kits comprising these compounds alone or in combination with other drugs or compounds aiming towards an improved treatment or prevention of the aforementioned diseases or syndromes in humans or animals.

### BACKGROUND OF THE INVENTION

Diabetes mellitus is a chronic disease characterized by hyperglycaemia and deranged glucose metabolism. Hyperglycaemia results from either deficiency of the glucose-lowering hormone insulin or from resistance of peripheral tissues to the effects of insulin together with inadequate levels of insulin secretion to compensate. There are two main forms of diabetes: type 1 and type 2 diabetes mellitus. Type 1 diabetes is an autoimmune disease that results in the permanent destruction of insulin producing beta cells of the pancreas. Normally, type 1 diabetes manifests during adolescence and is life threatening unless treated with exogenous insulin via injection. Type 2 diabetes is a metabolic disorder that is primarily characterized by peripheral insulin resistance, relative insulin deficiency, and mild hyperglycaemia at onset. In contrast to type 1 diabetes, type 2 diabetes may go unnoticed for years before diagnosis. Risk factors of type 2 diabetes include obesity, age, first degree relatives with type 2 diabetes, history of gestational diabetes, hypertension and hypertriglyceridaemia. The most prevalent factors driving the development of insulin resistance and type 2 diabetes are life style associated, the main risk factor being obesity. Around 90% of the patients with type 2 diabetes are overweight or obese. Increased fat mass, especially an excess of abdominal fat causes insulin resistance, insulin resistance places a greater demand on the pancreatic beta-cells to produce insulin and due to exhaustion of the pancreas, insulin production declines with age leading to the development of apparent diabetes. In developed countries, type 2 diabetes represents about 90% of all diabetes.
Ref: Report of World Health Organisation: Definition and diagnosis of diabetes mellitus and intermediate hyperglycemia. WHO/IDF consultation, WHO, Geneva, 2006

Diabetes mellitus is a growing health burden across the world. It is one of the most common diseases globally and among the leading causes of death in developed countries. At present, the three countries estimated to have the highest number of people with diabetes are India, China and the USA. Although the number of people with diabetes is already very high, numbers continue to increase at an alarming rate. The prevalence of diabetes worldwide is expected to double between 2000 and 2030 (2.8% in 2000 and minimum 4.4% in 2030). The total number of people with diabetes is projected to rise from 171 million in 2000 to at least 366 million in 2030 with the greatest relative increase anticipated in the developing countries in the Middle East, Africa and India. Although there is also a noticeable increase in type 1 diabetes, presumably due to changes in environmental risk factors, the “diabetes epidemic” is driven mainly by an increasing number of patients with type 2 diabetes. This is attributed to population growth, ageing, urbanisation and increased prevalence of obesity and physical inactivity. In some parts of the world overweight (Body Mass Index, BMI >25) and obesity (BMI >30) have increased to epidemic proportions in association with rapid cultural and social changes, including the excessive consumption of diets high in fat and protein. The human and economic costs of this epidemic are enormous. Weight-related escalating diabetes prevalence and cardiovascular disease, which is associated with diabetes, are expected to be the most significant public health concerns throughout this century and will lead to an immense financial burden. At present, the annual direct healthcare costs of diabetes are estimated to be at least between 153 and 286 billion dollars. In the light of such development, there is a big requirement for effective interventions including dietary and behavioural changes as well as pharmacological approaches.
Ref.: Zimmet P, Alberti K G M M, Shaw J: Global and societal implications of the diabetes epidemic. Nature 414, 782-787, 2001; Wild S, Roglic G, Green A, Sicree R, King H: Global prevalence of diabetes, estimates for the year 2000 and projections for 2030. Diabetes Care 27, 1047-1053, 2004

While established treatment regimens allow the diabetic patient an almost normal life for the short term, prolonged presence of the disease over time leads to serious damage of tissues, especially nerves and blood vessels. The resulting late complications of diabetes include coronary artery and peripheral vascular disease, cerebrovascular disease, diabetic neuropathy, diabetic foot, nephropathy and retinopathy. This causes cumulative proportions of disabilities and increased mortality. In virtually every developed society, diabetes is ranked among the leading causes of blindness, renal failure and lower limb amputation and about half of the money spent on diabetes care goes towards the costs of managing complications. The mechanisms by which diabetes leads to complications are not fully understood, but large studies have clearly confirmed that intensified therapy aiming at an early and stringent control of blood glucose reduces the incidence and severity of complications. Although early intense intervention increases the initial costs, the long term human and economic costs resulting from complications are decreased. This highlights the rationale not only for early lifestyle intervention but also for early pharmacotherapy and for the definition of ambitioned target levels of a near-normal control of blood glucose. As a consequence, any new drug or drug combination that contributes to further improvement and optimisation of blood glucose control is a valuable tool to prevent late complications and to reduce the medical and economic burden of diabetes.
Ref.: DCCT Research Group: The effect of intensive treatment of diabetes on the development and progression of long-term complications insulin-dependent diabetes mellitus, N Engl J Med 329, 977-986, 1993; UK Prospective Diabetes Study (UKPDS) Group: Intensive blood-glucose control with sulphonylureas or insulin compared with conventional treatment and risk of complications in patients with type 2 diabetes (UKPDS 33). Lancet 352, 837-853, 1998 UK Prospective Diabetes Study Group, UKPDS: Effect of intensive blood-glucose control with metformin on complications in overweight patients with type 2 diabetes (UKPDS 34). Lancet 352, 854-65, 1998

Both, type 1 and 2 diabetes mellitus have no medically proven cure and, hence, the main goal of treatment is the reduction of morbidity and mortality from complications. This can be achieved through effective treatment of hyperglycaemia with HbA_{1c} as a valuable readout parameter for glucose control over time. In type 1 diabetes, treatment with exogenous insulin is essential and, hence, improvement of blood glucose control is mainly reached by more sophisticated insulin injection regimens. Type 2 diabetes is a chronic, progressive disease and its pathophysiology varies markedly more among patients than that of type 1 diabetes. This suggests versatile strategies for prevention, diagnostic screening and treatments of type 2 diabetes. Besides lifestyle management, blood pressure control, cardiovascular risk protection and diabetic complications screening, pharmaceuticals are needed to optimise the treatment and outcome. In this context, a variety of oral drugs is available for the treatment of type 2 diabetes. These drugs affect blood glucose via different mechanisms of action. According to the global guidelines for type 2 diabetes from the International Diabetes Foundation treatment recommendations are as follows: The insulin sensitising biguanide metformin is the drug of choice for first-line oral therapy of type 2 diabetes. Its major effect is to lower glycaemia by decreasing the hepatic glucose output. When metformin fails to sufficiently control blood glucose concentrations, sulfonylureas and/or PPARγ agonists should be added. Whereas sulfonylureas enhance insulin secretion, PPARγ agonists (thiazolidinediones) increase the sensitivity of muscle, fat, and liver to insulin. Further additional treatment options are α-glucosidase inhibitors, exenatide, glinides, or pramlintide. α-Glucosidase inhibitors reduce the rate of digestion of polysaccharides in the small intestine, which delays glucose absorption from the intestine and lowers postprandial plasma glucose concentrations. Glinides stimulate insulin secretion similar to sulfonylureas but with shorter half life. Exenatide (glucagon-like peptide 1 agonist) potentiates glucose mediated insulin secretion and pramlintide (amylin agonist) slows gastric emptying and inhibits glucagon production. If drugs and lifestyle-interventions are unable to maintain blood glucose control, insulin therapy is required at the late stage of the disease development.
Ref.: International Diabetes Foundation, Clinical Guidelines Task Force: Global guideline for type 2 diabetes, 2005. www.idf.org/webdata/docs/IDF%20GGT2D.pdf. Nathan D M Buse J B, Davidson M B, Heine R J, Holman R R, Sherwin R, Zinman B: Management of hyperglycemia in type 2 diabetes: a consensus algorithm for the initiation and adjustment of therapy: a consensus statement from the American Diabetes Association and the European Association for the Study of Diabetes. Diabetes Care 29, 1963-1972, 2006

Apart from varying pathophysiology among patients, type 2 diabetes is a progressive disease with worsening glycaemia over time. Since monotherapies fail to reach glycaemic goals in almost three out of four patients, more than one medication will be necessary for the majority of patients over time and combinations of drugs with different mechanisms of action will encounter best treatment success in most cases. Nevertheless, numerous medications in several combinations still fail to achieve and maintain glycaemic levels to provide optimal health care status for most individual patients, which emphasises the continuing requirement for new and better drugs. Apart from unsatisfactory performance with respect to the treatment targets in glycaemic control, the prescription of many glucose lowering drugs is limited by concerns about adverse effects. Metformin, recommended for first-line oral therapy of type 2 diabetes, is relatively well tolerated. The most common adverse effects of metformin are gastrointestinal problems, but metformin has also been associated with lactic acidosis as an extremely rare but also an extremely dangerous adverse effect. Gastrointestinal problems are even much more common for other classes of drugs for type 2 diabetes. At least one third of the patients taking glucosidase inhibitors, exenatide or pramlintide are afflicted by gastrointestinal side effects, which are a frequent cause for discontinuation of treatment. Gastrointestinal effects are not a problem with sulfonylureas and glinides, but these drugs act by inducing insulin secretion and bear the risk of hypoglycaemia, which in extreme cases can be life threatening. And finally, the thiazolidinediones, which initially produced high expectations because of their favourable insulin sensitising mechanism of action, revealed to induce fluid retention and have recently even been suspected of increasing myocardial infarction and the risk of death from cardiovascular causes. Unsatisfactory efficacy in reaching the treatment goals, frequent problematic adverse effects and in many cases high costs are therefore unresolved problems in the present pharmaceutical treatment options for type 2 diabetes. Considering available pharmaceutical tools in the light of the alarming epidemiology of type 2 diabetes, an urgent need is obvious for new drugs with a better therapeutic index, i.e. with an improved relation of efficacy per adverse effects.
Ref.: Nathan D M, Buse J B, Davidson M B, Heine R J, Holman R R, Sherwin R, Zinman B: Management of hyperglycemia in type 2 diabetes: a consensus algorithm for the initiation and adjustment of therapy: a consensus statement from the American Diabetes Association and the European Association for the Study of Diabetes. Diabetes Care 29, 1963-1972, 2006; Nissen S E, Wolski K: Effect of rosiglitazone on the risk of myocardial infarction and death from cardiovascular causes. N Engl J Med 356, 2457-2471, 2007

In search of novel glucose lowering agents, early preclinical examination and characterisation is usually based on the study of rodent strains with metabolic deviations resembling the diabetic state. In such animals, glucose homeostasis is usually charged by postprandial glycaemia and by a glucose tolerance test (GTT), which determines the increase in blood glucose after administration of a glucose solution. In the GTT, glucose can be administered intravenously (IVGTT), intraperitoneally (IPGTT) or orally (OGTT), the latter being the most physiological approach. Rodents most frequently used as models for type 2 diabetes include such, in which increased glycaemia is due to a genetic defect, to dietary intervention or to the administration of toxic pharmacological agents. Each specific approach has advantages and limitations. Commonly used genetic models are rats and mice afflicted by a gene defect that causes overeating and severe obesity (e.g., ZDF rats, db/db mice). In these animals, very severe insulin resistance is the driving force behind the development of hyperglycaemia and, hence, they are very responsive to some agents that act via insulin sensitisation. This reasonably mimics the situation in extremely obese patients with type 2 diabetes, but the predominance of insulin resistance often makes it difficult to demonstrate in such models the glucose lowering action of drugs, which act via mechanisms other than insulin sensitisation. Other prevalently used models are rodents injected with agents that destroy insulin producing cells (streptozotocin, alloxan) and, if dosed appropriately, cause relative insulin deficiency. However, this model lacks the component of primary insulin resistance, which is a crucial characteristic of type 2 diabetes. Dietary models, in particular animals fed with a diet of very high fat content (high fat-diet, HFD) simulate better the pathogenesis of type 2 diabetes in the prevalent overweight patient. Since the degree of metabolic derangement remains limited, these models are comparable only with the early stages of the development of type 2 diabetes. There are strain differences regarding the extent of the HFD-induced derangement of glucose homeostasis with, e.g., C57/BL mice being more susceptible to HFD-induced metabolic derangements than other strains. The degree and the characteristics of the derangement can also be modulated by the diet composition. Usually, HFDs have a fat content around 60% (of calories) and contain carbohydrates and protein at a rate comparable to humans eating too much fat. Alternative HFDs are almost completely free of carbohydrates, which has the advantage of leading to more severe metabolic consequences within a shorter period of time, but mimics the situation in obese patients less appropriately.
Ref.: Surwit R S, Kuhn C M, Cochrane C, McCubbin J A, Feinglos M N: Diet-induced type II diabetes in C57BL/6J mice. Diabetes 37, 1163-1167, 1988; Winzell M S, Ahrén B: The high-fat diet-fed mouse: a model for studying mechanisms and treatment of impaired glucose tolerance and type 2 diabetes. Diabetes 53 (Suppl 3), S215-219, 2004 Burcelin R, Crivelli V, Dacosta A, Roy-Tirelli A, Thorens B: Heterogeneous metabolic adaptation of C57BL/6J mice to high-fat diet. Am J Physiol 282, E834-E842, 2002

In summary, there is still an unfulfilled need for compounds, compound combinations and therapies that may be used to overcome the aforementioned set-backs of state of the art diabetic treatments. The present invention is directed to these, as well as other important ends.

Surprisingly, it could be shown within the scope of this invention that the therapeutic use of novel substituted octahydroquinolizines as drugs in the therapeutic fields described above crucially depends upon their distinct chemical nature, particularly their substitution pattern. Thus, although chemically similar in the backbone framework, specific changes in structure result in dramatic changes in the pharmaceutical usefulness of different octahydroquinolizine derivatives. This includes, but is not limited to, for example the structural changes regarding the stereochemistry, the positioning of substituents on the backbone and their spacial properties, the acidic/basic properties of substituents, the incorporation of aromatic or non-aromatic groups in specific positions and the conformational flexibility of the various substitutions linked to the octahydroquinolizine backbone.

As compared to formerly published octahydroquinolizines [WO2007/050802 A (Adolor Corp [US], Dolle Roland E [US], Le Bourdonnec Bertrand [US], 3 May 2007); Kubo H. et al., Biol. Pharm. Bull. 23(9), 1114-1117 (2000)] the novel compounds invented here mark a substantial superiority in the biological activity proven in animal models which are targeted towards the treatment of diabetes and the aforementioned diseases. These advantages include for example, but are not limited to, a superior dose-activity relationship and/or pharmacological profile or total lack or a significant reduction of acute toxicity in murine diabetic models and/or total lack or significant reduction of an unfavourable adverse effect profile in rodent or non-rodent animal models. Compounds showing adverse effects in animal models normally are excluded from clinical development and they are therefore not suitable for use in human treatment of diabetes and related diseases.

The compounds disclosed in this invention allow for a novel treatment or prevention of diabetes and related diseases. In particular, due to their particular mode of action, which is unprecedented in diabetic therapy, the disclosed compounds convey their therapeutic superiority devoid of side effects which significantly hamper the therapeutic benefit of the state of the art antidiabetic treatments. This includes, but is not limited to, side effects known to date as for example: intestinal side effects as observed in the course of the therapeutic use of e.g. glucosidase inhibitors or glucagon-like peptide 1 (GLP-1) mimics like exenatide; life threatening hypoglycemia documented with the use of insulin and/or insulin secreting drugs like sulfonylureas; dangerous lactic acidosis of which patients may suffer treated with biguanides; unwanted gastrointestinal or immune modulating side effects of state of the art drugs which act via the inhibition of dipeptidyl peptidase IV as for example the gliptins.

Therefore, the compounds disclosed in this invention mark an unpredicted and substantial progress in the aforementioned therapeutic use.

### SUMMARY OF THE INVENTION

The present invention is generally directed to substituted octahydroquinolizine derivatives, pharmaceutical compositions containing these compounds and methods of their pharmaceutical use.

In one embodiment, the invention is directed to compounds of formula I

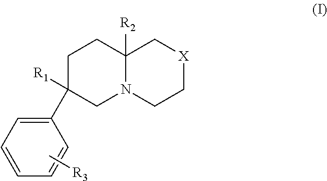

R¹=C₁-C₆ alkyl, Phenyl, substituted Phenyl
R²=H, C₁-C₆ alkyl, alkyl-cycloalkyl
R³=(R³¹)ₖ wherein k=0, 1, 2, 3
   R³¹=H, F, Cl, Br, CF₃, C₁-C₆ alkyl
(R³²)ₖ wherein k=4, 5
   R³²=H, F
X=Carbonyl, R⁹, CR⁴CN, CHR⁵, CH(COH(CH₃)₂), CR⁴(OR⁶), CR⁶(OR⁴), CR⁶benzyloxy, CR⁶(2-methoxyethoxy), CR⁶[(2-methoxyethoxy)methoxy], CR⁶[(2-methoxyethoxy)ethoxy], CR⁴(CO)OR⁴, CR⁴(CO)N(R⁴)₂, CR⁴(CO)R⁵, CR⁴(CO)R⁴ CR⁴(CH₂)ₖ(Y)ₘ(CH₂)ₙZ, C(OR⁴)(CH₂)ₖ(Y)ₘ(CH₂)ₙZ, C(Otrimethylsilyl)(CH₂)ₖ(Y)ₘ(CH₂)ₙZ
   wherein k=1, 2, 3, 4; m=0, 1; n=0, 1, 2, 3
      Y=CR⁴, R⁶, 1,1-cyclopentyl, 1,1-cyclohexyl,
      Z=R⁵, R⁶, R⁷, R⁸, CN, (CO)OR⁶, (CO)R⁴, OR⁶, OR⁷, O(CO)R⁵, (CO)R⁵, (CO)R⁸, O(CH₂)_{2 or 3}R⁵, O(CH₂)_{2 or 3}R⁶, O(CH₂)_{2 or 3}R⁷, 0 (CH₂)_{2 or 3}R⁸, O(CH₂)_{2 or 3}OR⁶, O(CH₂)_{2 or 3}OR⁷
         NR⁴(CO)OR⁶, NR⁴(CO)R⁵
         NR⁴(CH₂)_{2 or 3}R, NR⁴(CH₂)_{2 or 3}R⁶, NR⁴(CH₂)_{2 or 3}R⁷, NR⁴(CH₂)_{2 or 3}R⁸, NR⁴(CH₂)_{2 or 3}OR⁶, NR⁴(CH₂)_{2 or 3}OR⁷
            wherein:
R⁴=H, C₁-C₆ alkyl
R⁵=

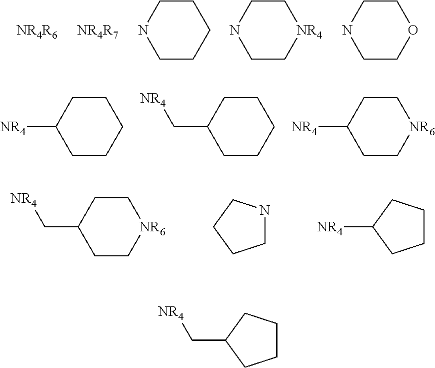

R⁶=H, C₁-C₆ alkyl, isopropyl, isobutyl, secbutyl, t-Butyl, cyclopentyl, cyclohexyl, cycloheptyl, cyclopentylmethylen, cyclohexylmethylen
R⁷=phenyl, monofluorophenyl, difluorophenyl, trifluorophenyl, trifluoromethylphenyl, chlorophenyl, dichlorophenyl, monofluoro-monochlorophenyl, difluoro-monochlorophenyl, monofluoro-monomethylphenyl, methylphenyl, dimethylphenyl
R⁸=

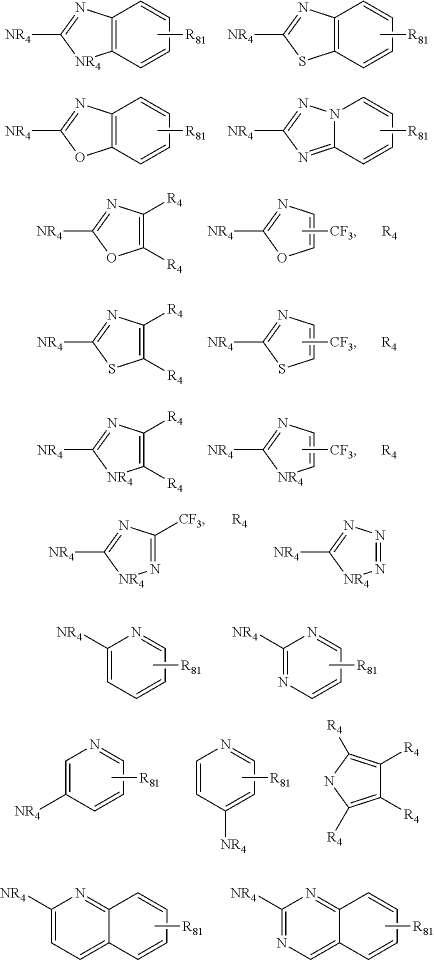

R⁸¹=(F, Cl, CF₃, C₁-C₆ alkyl)_{0, 1 or 2} independently of each other
R⁹=

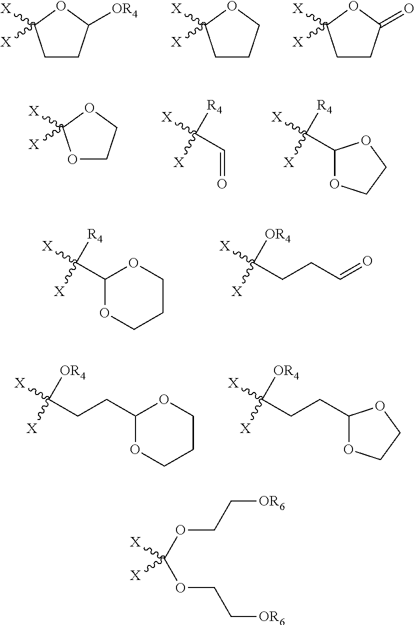

In another embodiment, the invention is directed to compounds of formula II:

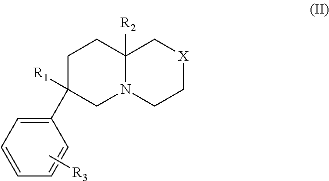

R¹=Methyl, Phenyl
R²=H, Methyl
R³=H, F, Cl, CF₃, difluoro, trifluoro, dichloro, monofluoro-monochloro, methyl, dimethyl, monofluoro-monomethyl
X=Carbonyl, R⁹, CR⁴CN, CHR⁵, CH(COH(CH₃)₂), CR⁴(OR⁶), CR⁶(OR⁴), CR⁶benzyloxy, CR⁶(2-methoxyethoxy), CR⁶[(2-methoxyethoxy)methoxy], CR⁶[(2-methoxyethoxy)ethoxy], CR⁴(CO)OR⁴, CR⁴(CO)N(R⁴)₂, CR⁴(CO)R⁵, CR⁴(CO)R⁴ CR⁴(CH₂)ₖ(Y)ₘ(CH₂)ₙZ, C(OR⁴)(CH₂)ₖ(Y)ₘ(CH₂)ₙZ, C(Otrimethylsilyl)(CH₂)ₖ(Y)ₘ(CH₂)ₙZ
   wherein k=1, 2, 3; m=0, 1; n=0, 1, 2,
      Y=CR⁴, R⁶, 1,1-cyclopentyl, 1,1-cyclohexyl,
      Z=R⁵, R⁶, R⁷, R⁸, CN, (CO)OR⁶, (CO)R⁴, OR⁶, (CO)R⁵, (CO)R⁸, NR⁴(CO)R⁵
         wherein:
R⁴=H, C₁-C₆ alkyl
R⁵=

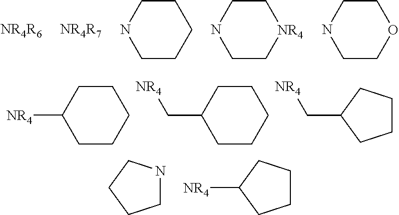

R⁶=H, C₁-C₆ alkyl, isopropyl, isobutyl, secbutyl, t-Butyl, cyclopentyl, cyclohexyl,
R⁷=phenyl, monofluorophenyl, difluorophenyl, trifluorophenyl, trifluoromethylphenyl, chlorophenyl, dichlorophenyl, monofluoro-monochlorophenyl, difluoro-monochlorophenyl, monofluoro-monomethylphenyl, methylphenyl, dimethylphenyl
R⁸=

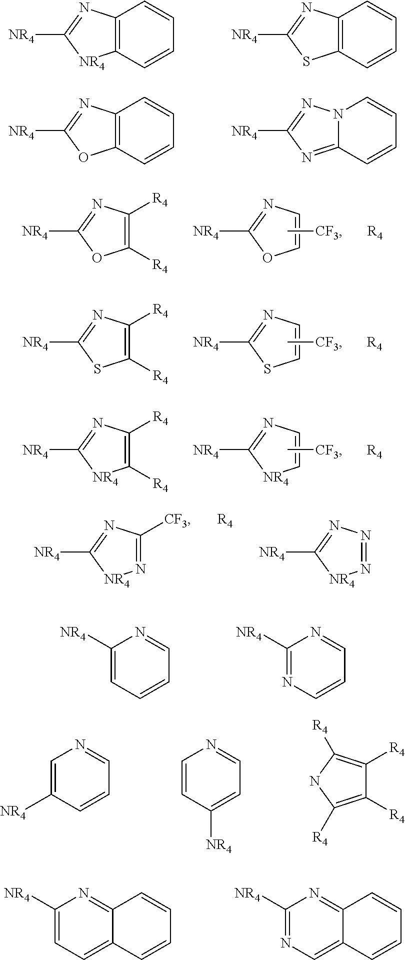

R⁹=

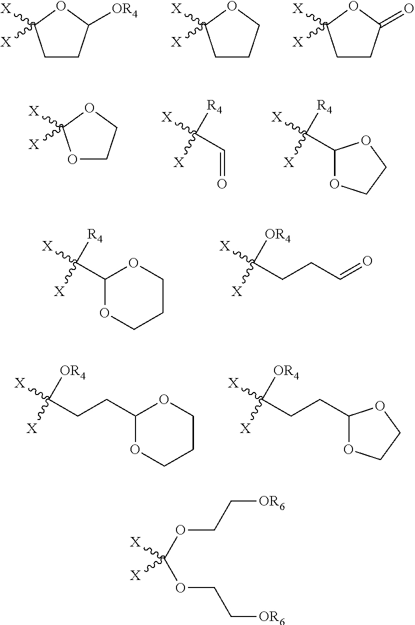

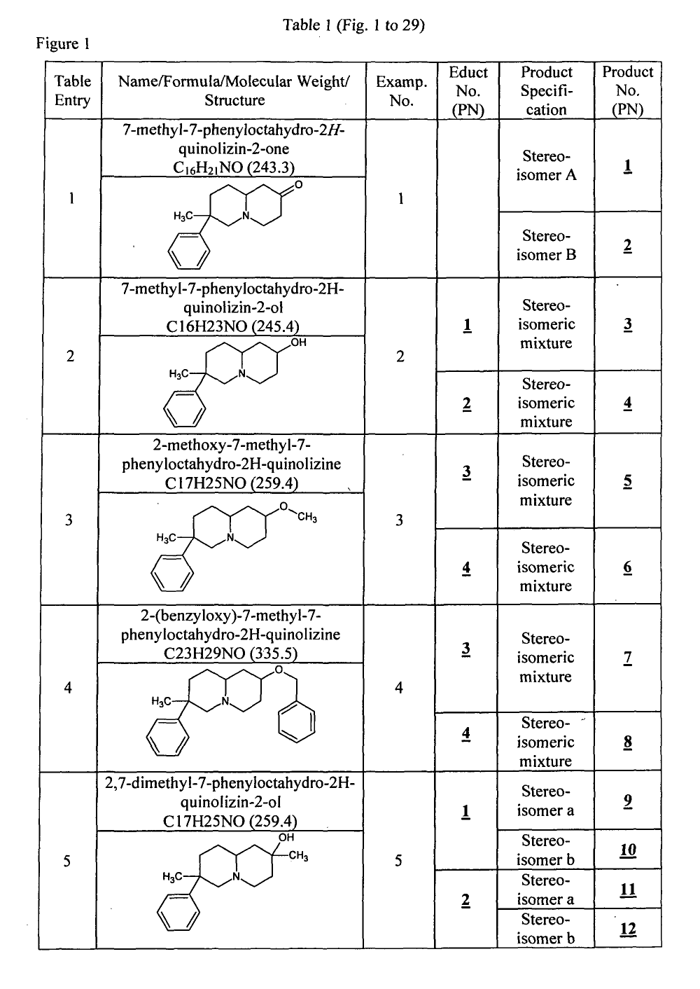

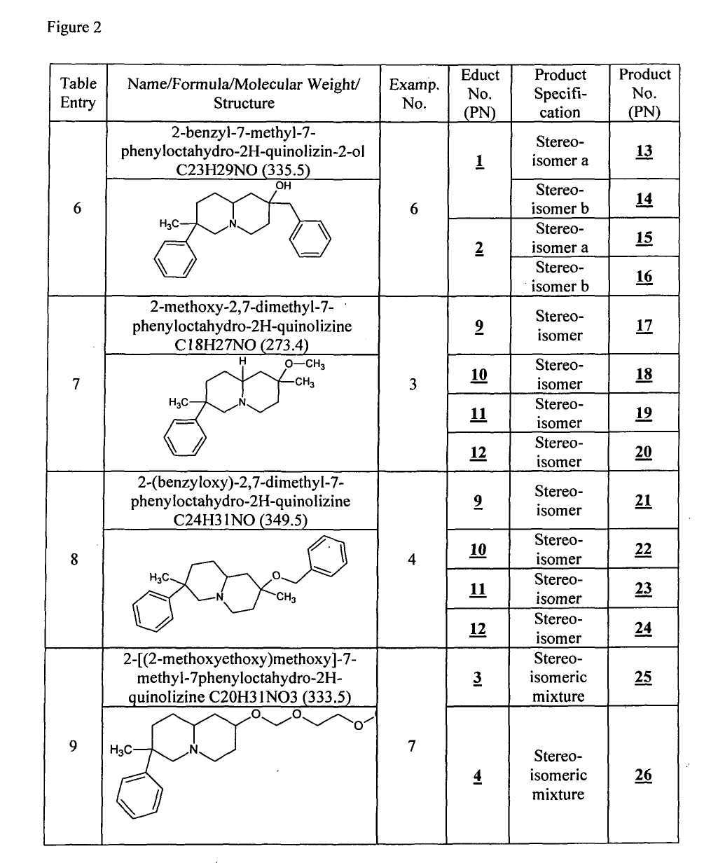

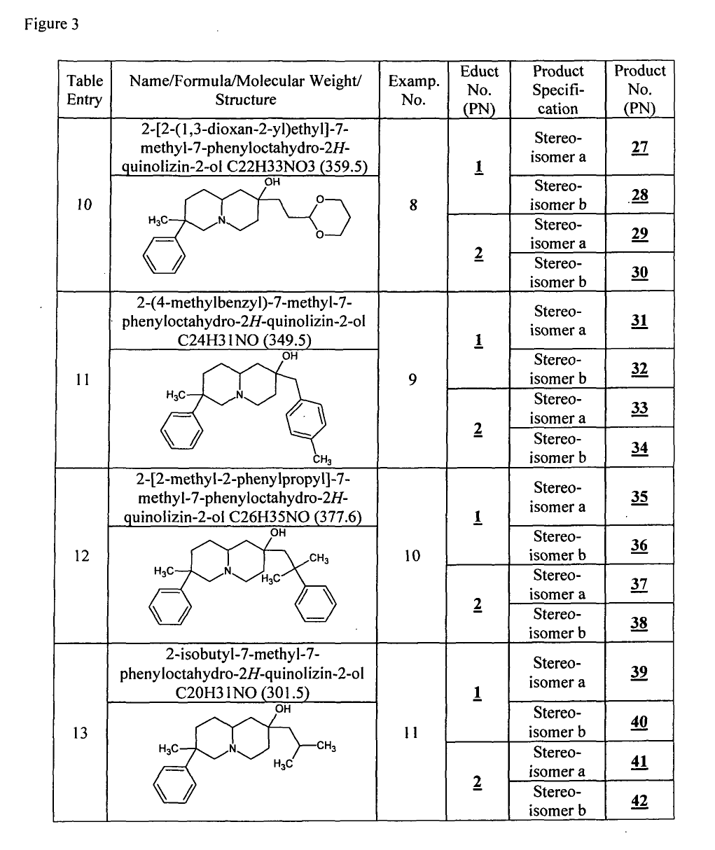

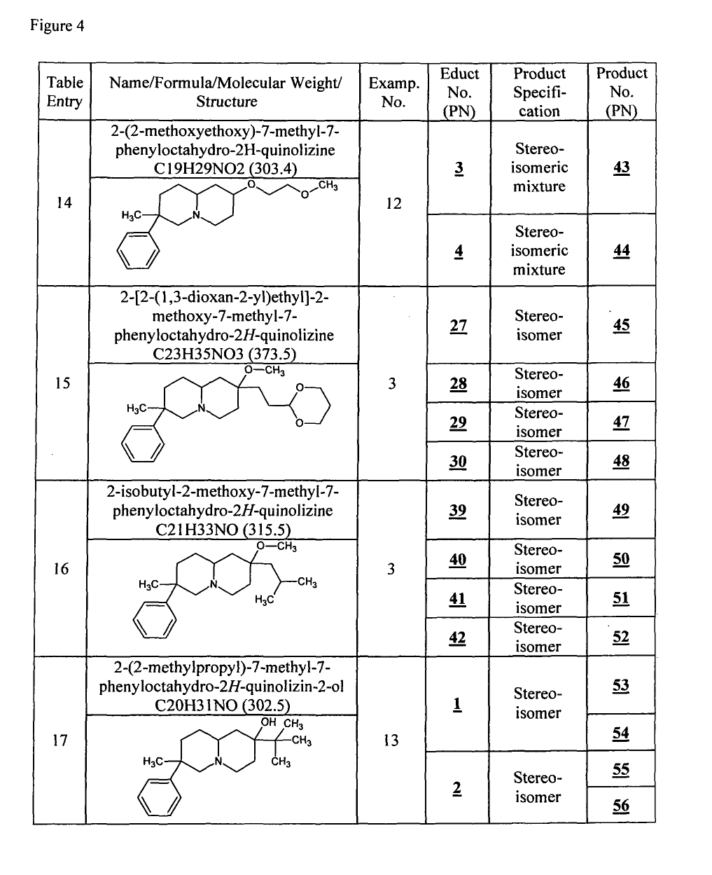

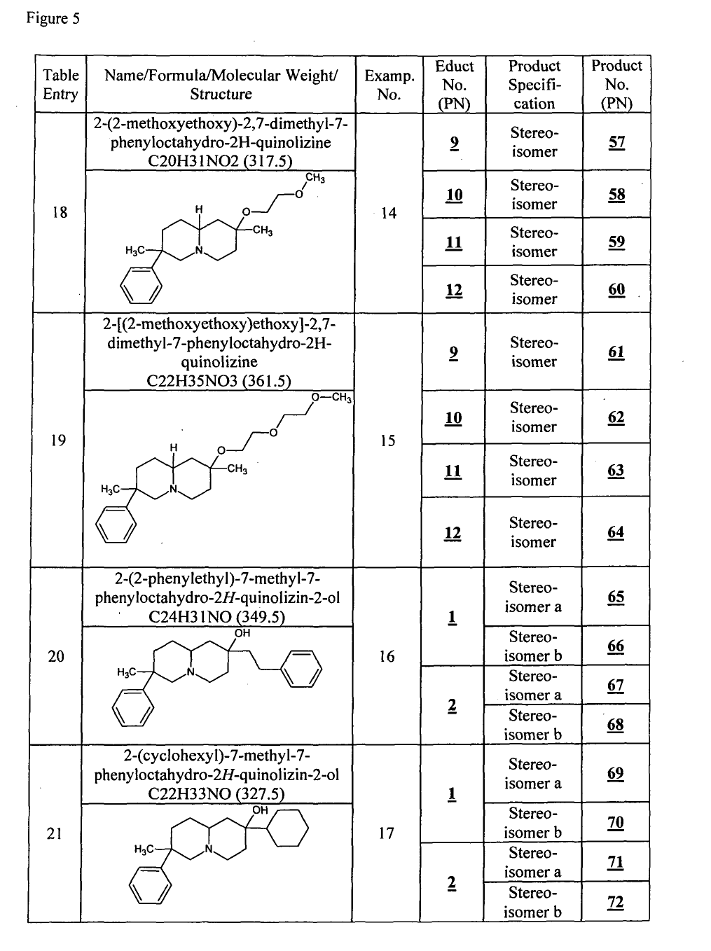

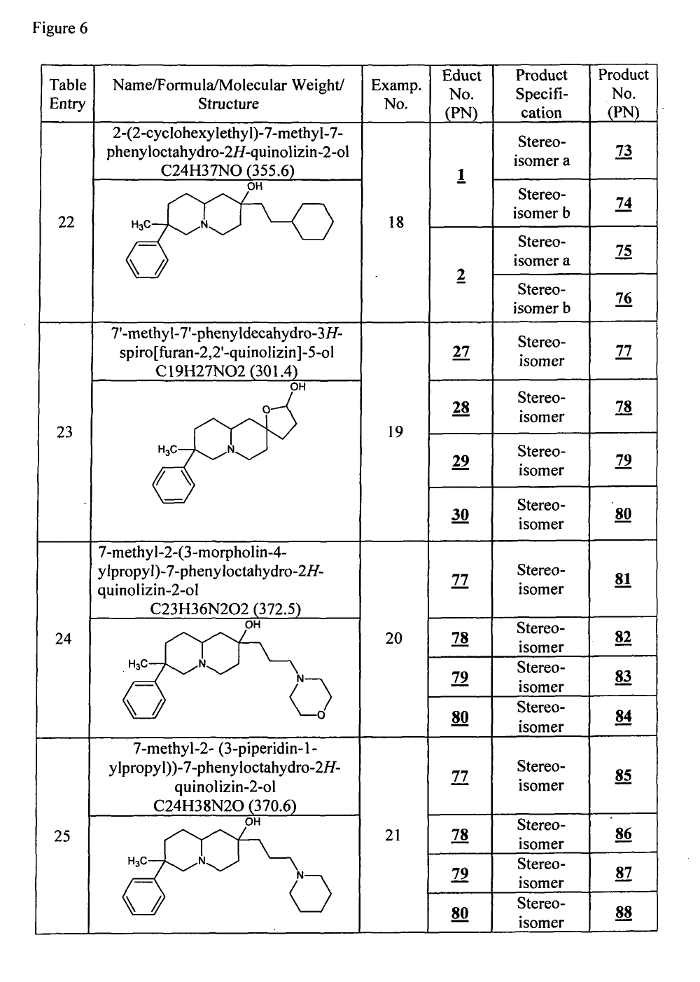

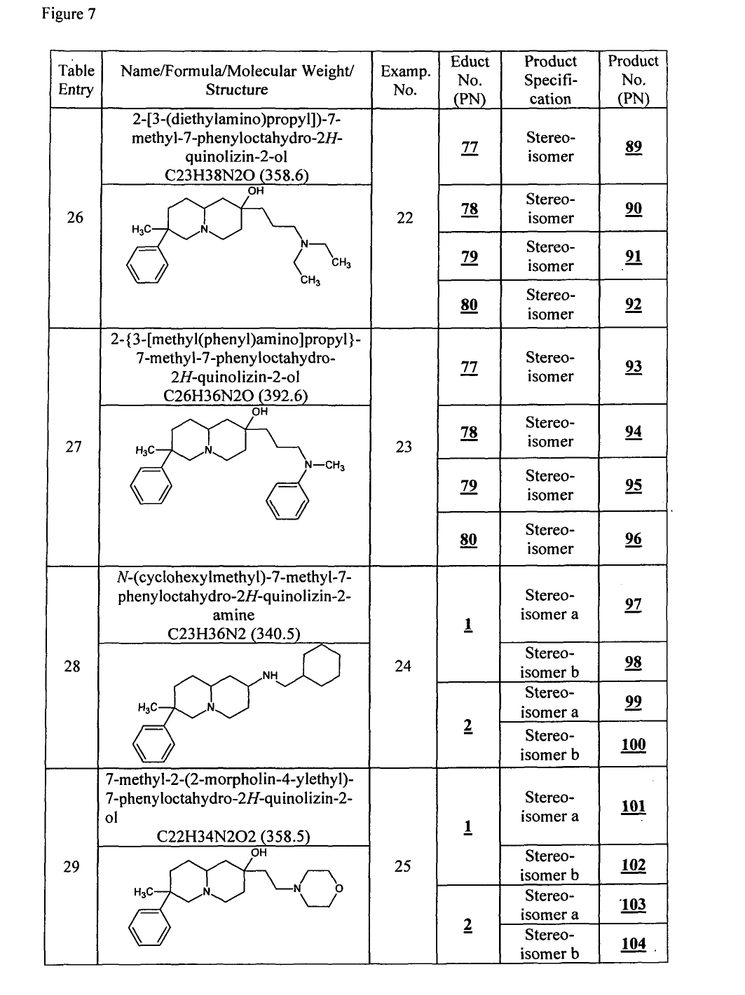

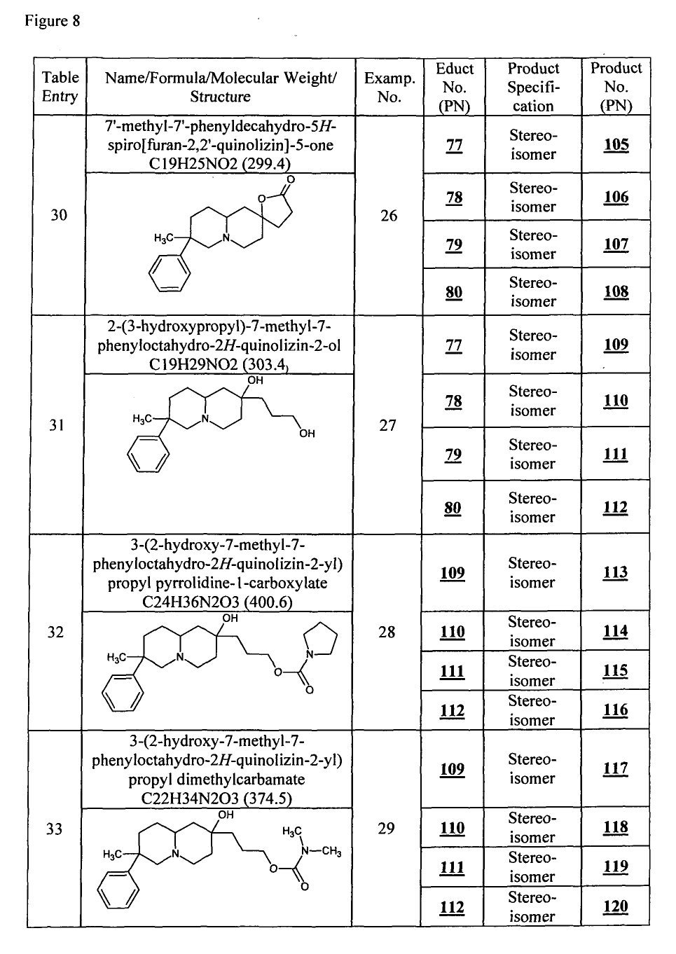

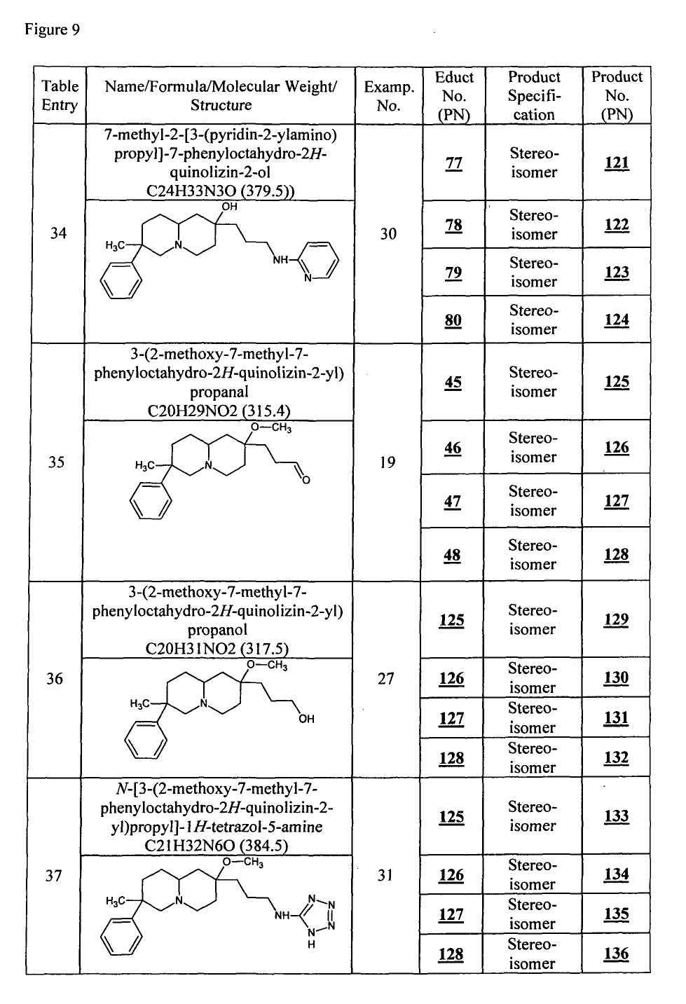

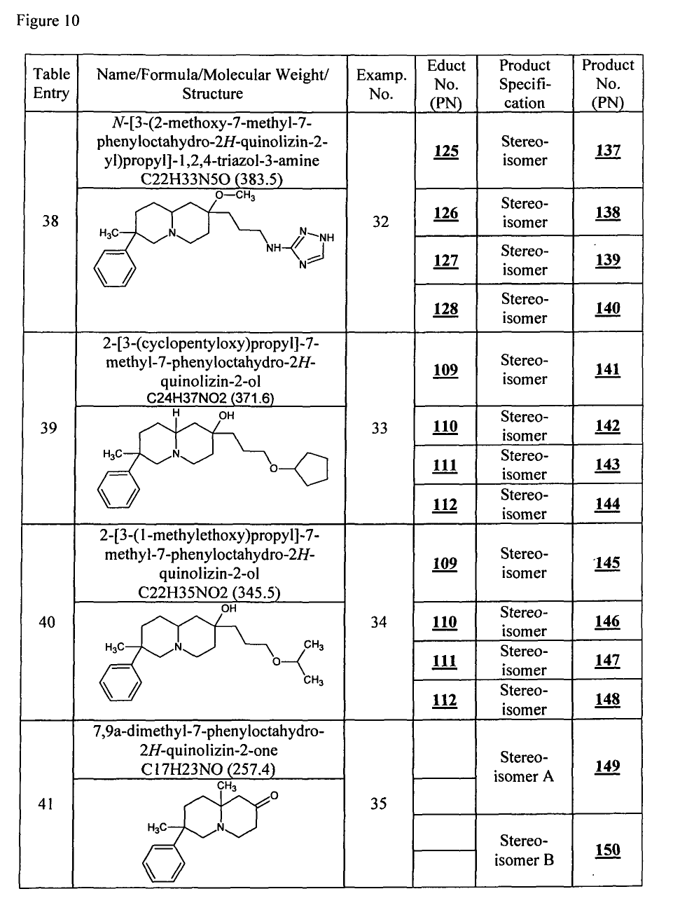

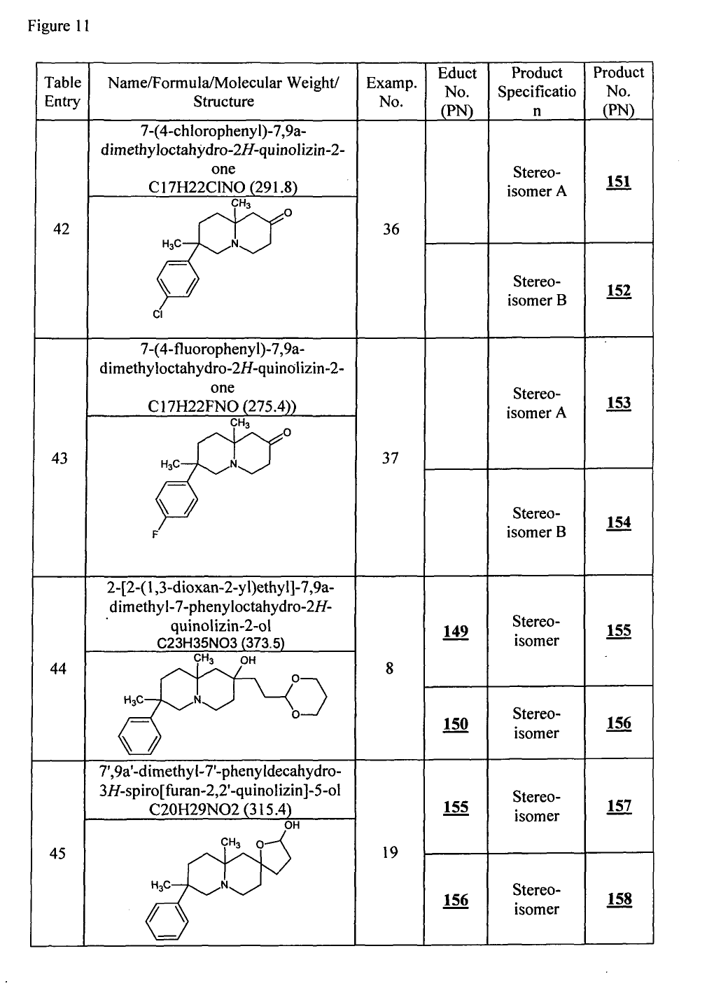

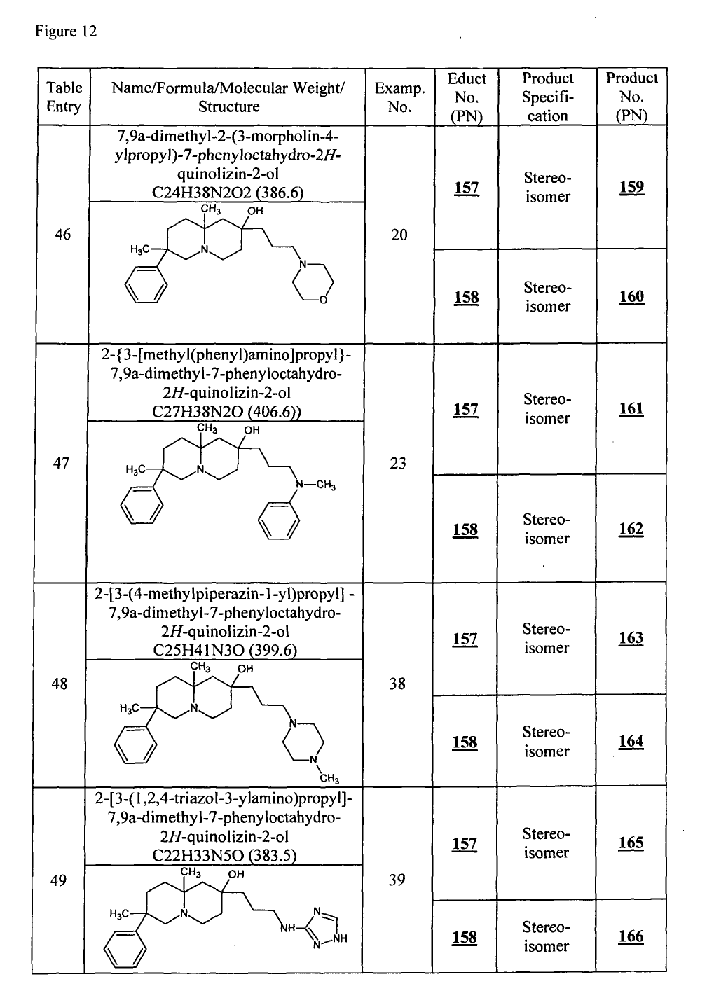

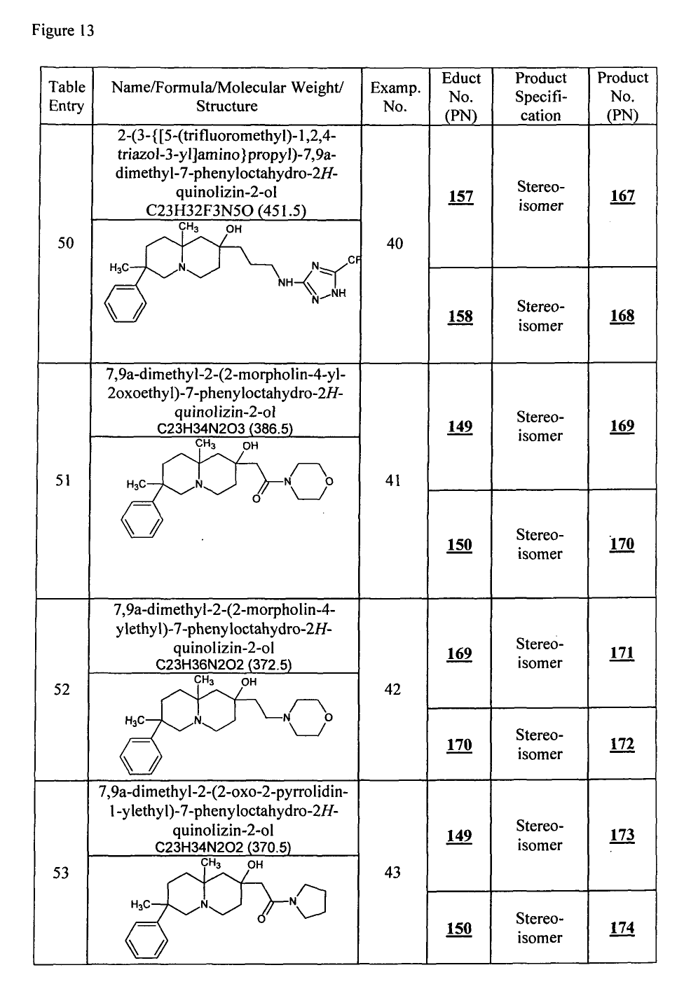

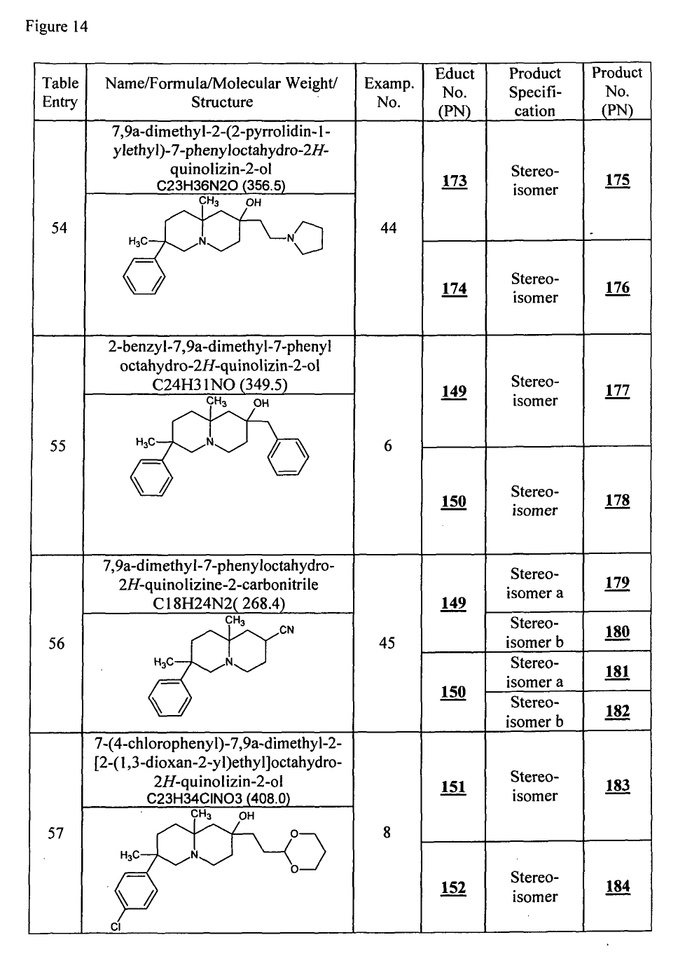

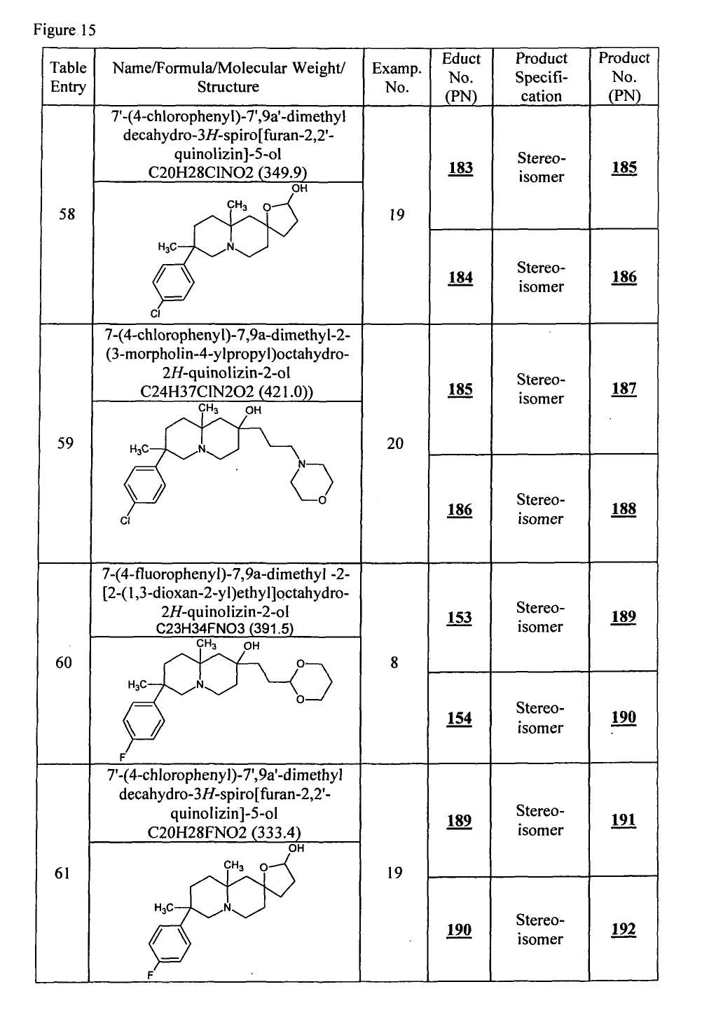

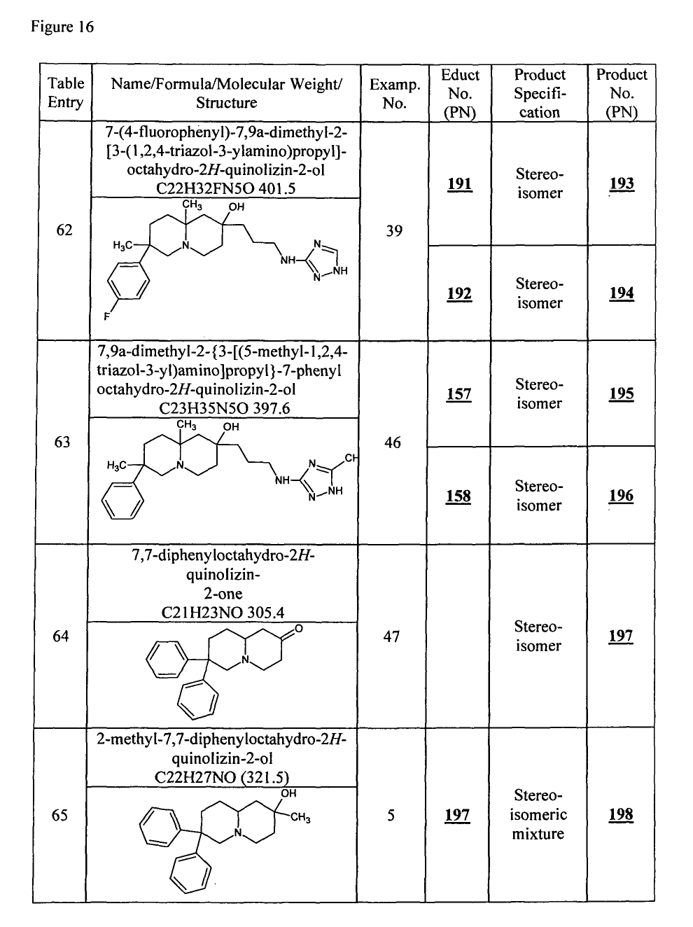

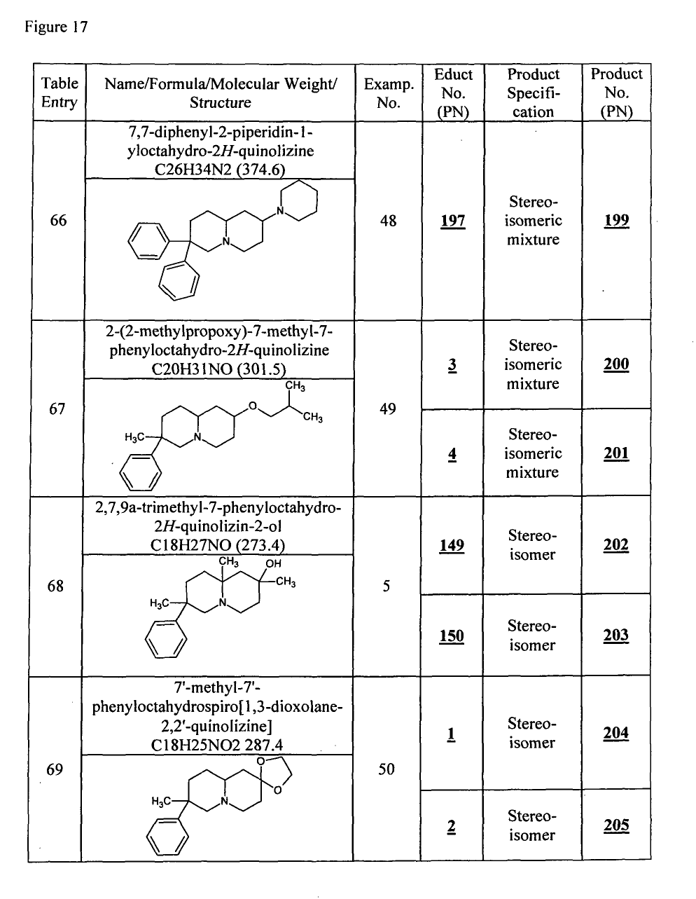

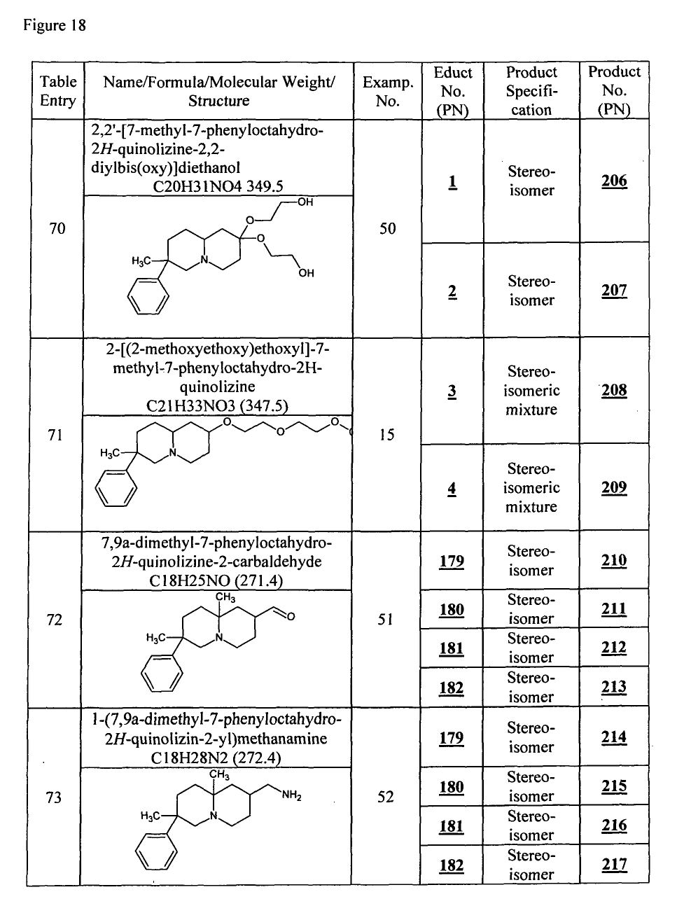

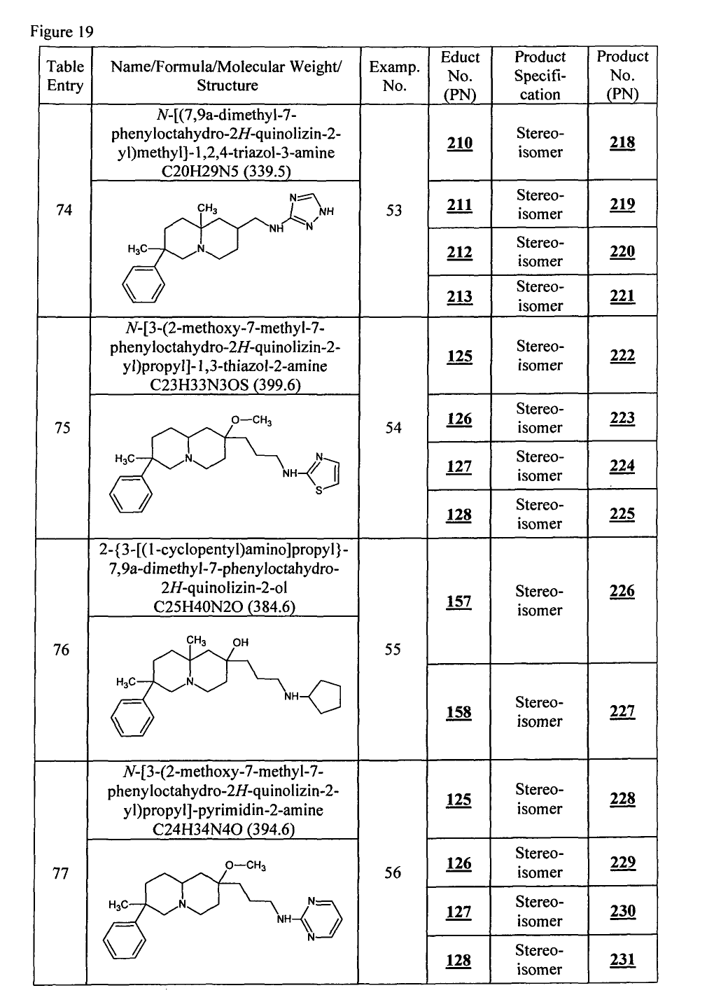

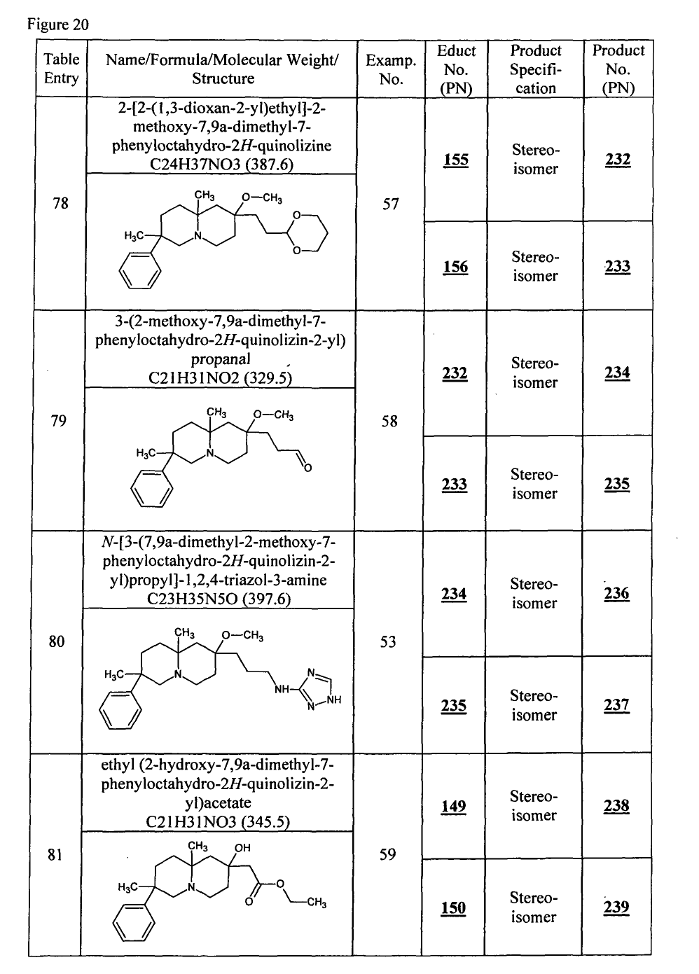

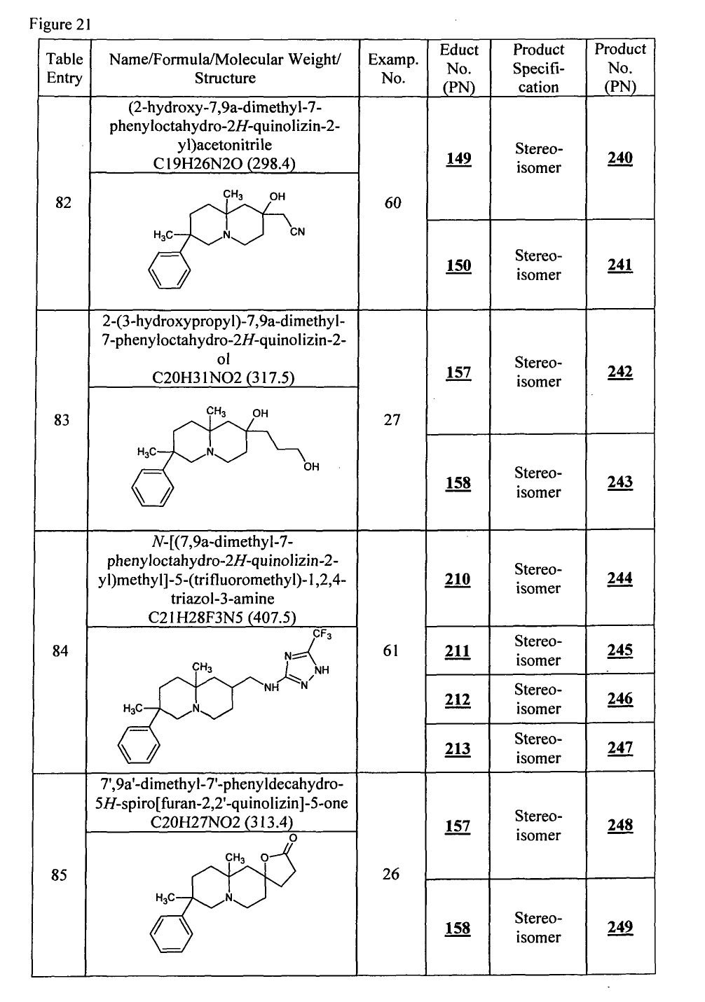

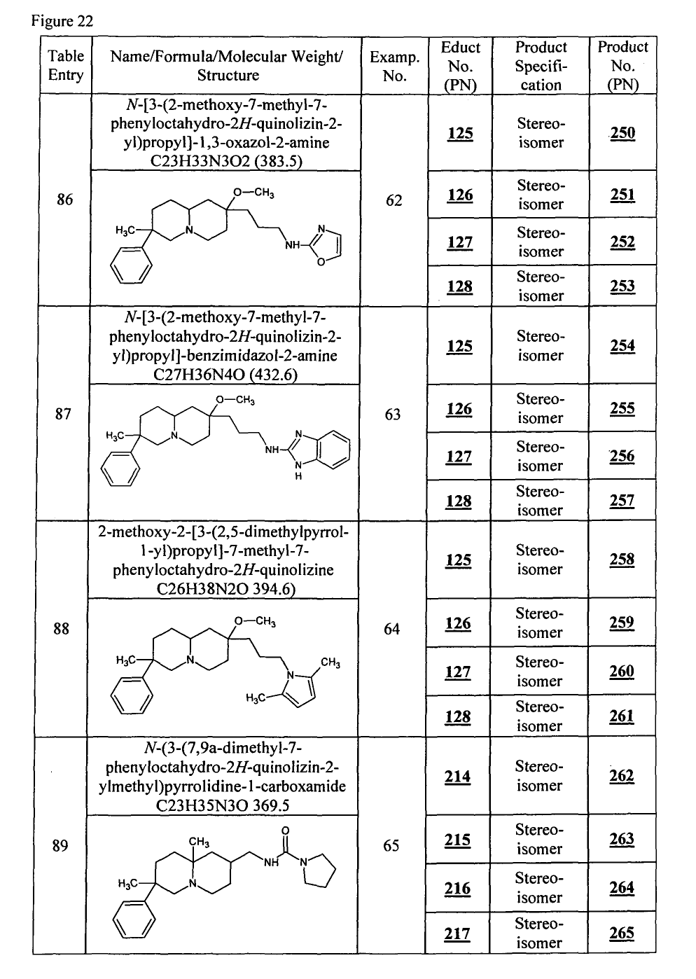

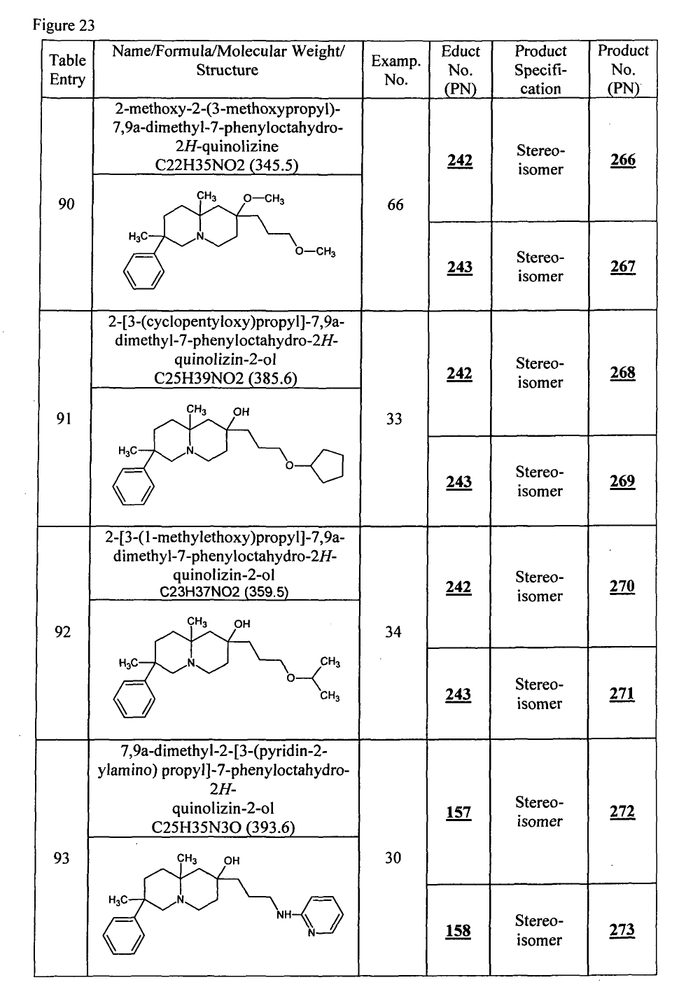

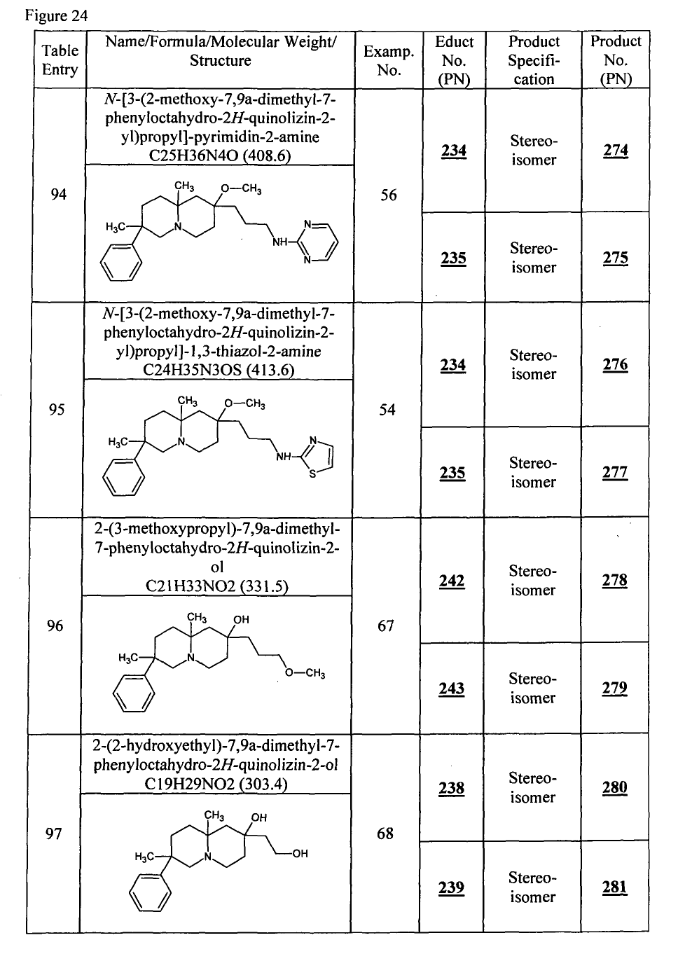

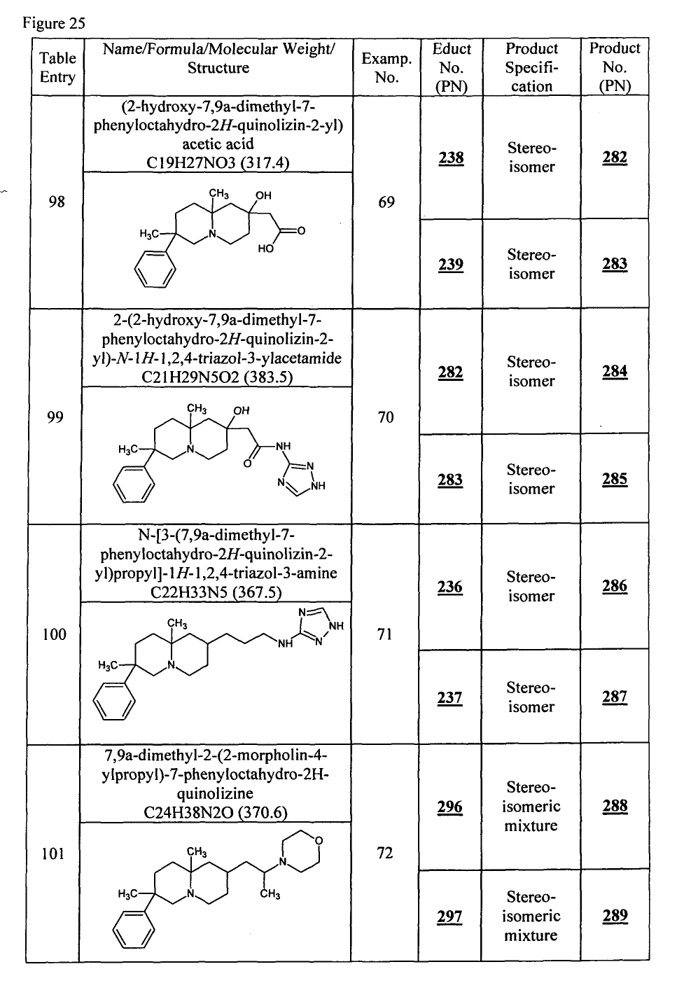

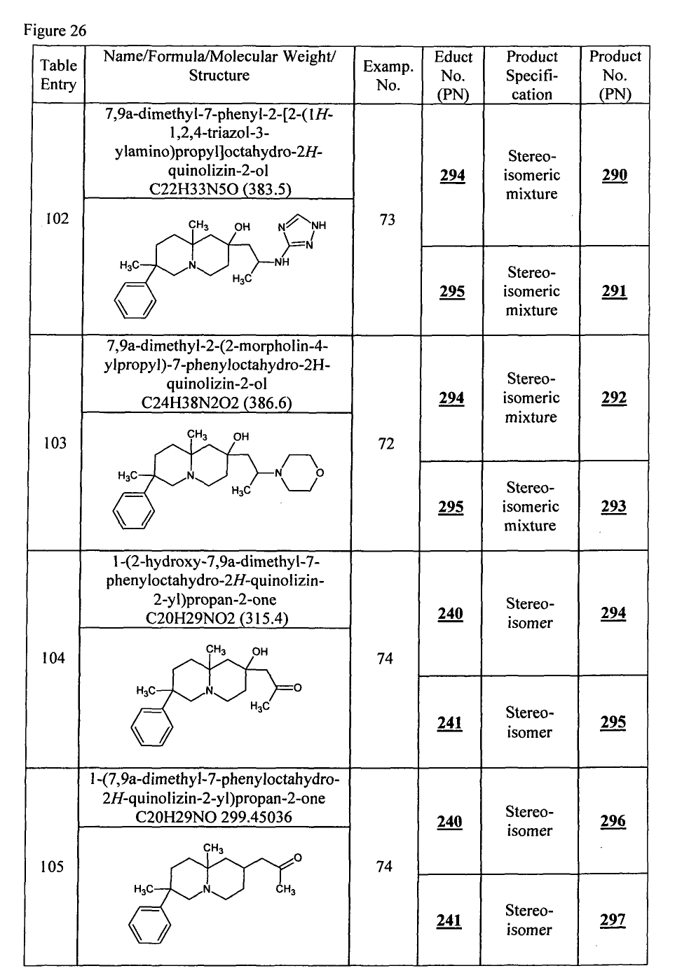

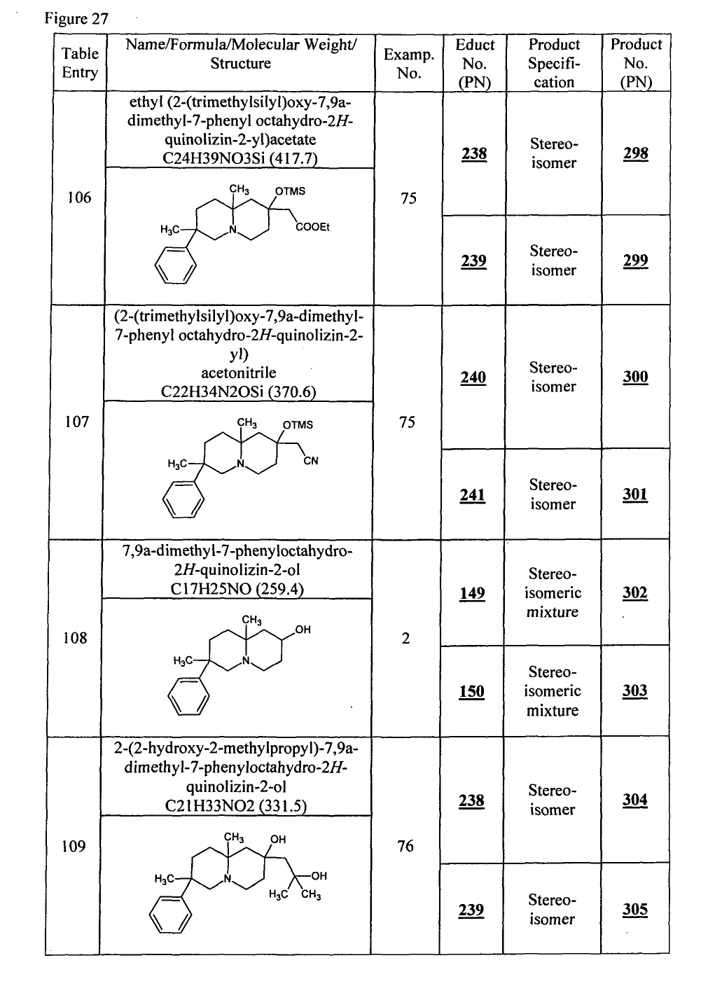

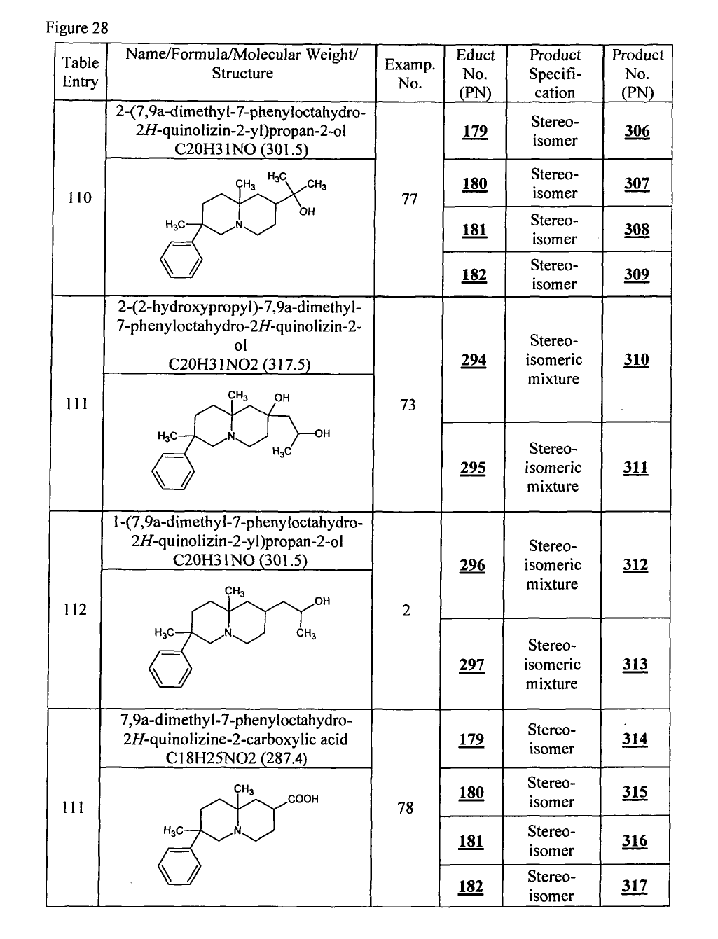

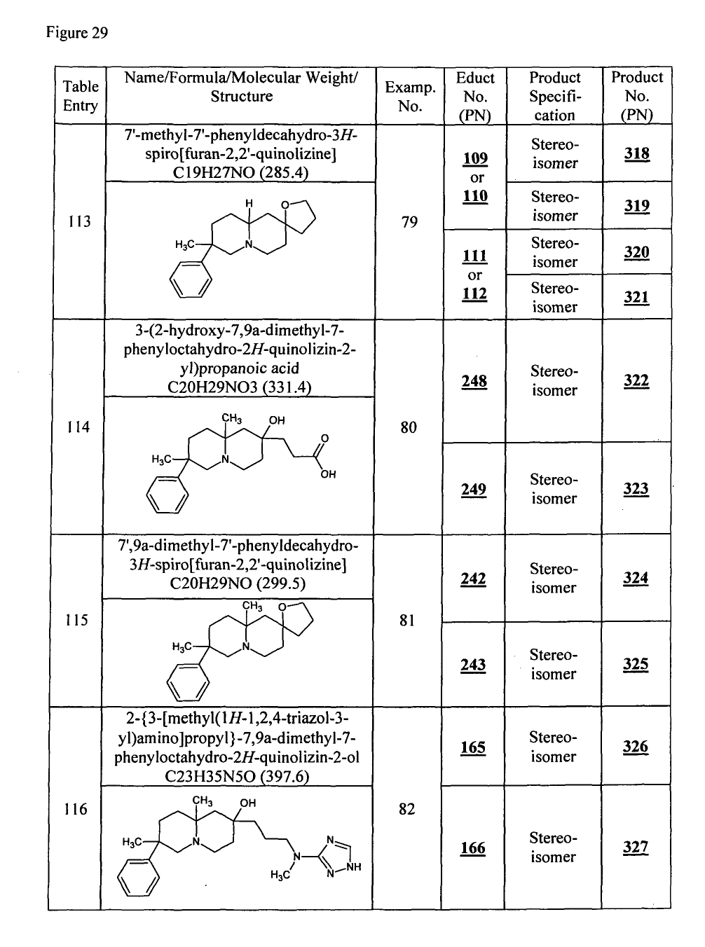

In still another embodiment, the invention is directed to compounds of formula I and II as listed in Table 1 (FIGS. 1 to 29).

A still another embodiment comprises the more preferred embodiments. More preferred embodiments are the compounds which are listed in Table 1 (FIGS. 1 to 29), marked with an asterix (*) and which have the following product numbers (PN): 29, 79, 83, 111, 131, 135, 139, 143, 147, 156, 158, 160, 162, 166, 168, 190, 194, 224, 230, 249, 287, 320.

A further aspect of the invention is a pharmaceutical composition containing a compound of formula I or II as drug substance.

A further aspect of the invention is the use of a compound of formula I or II according to claims 1 to 3 for the manufacture of a pharmaceutical composition for the treatment or prevention of diabetes mellitus.

A further aspect of the invention is the use of a compound of formula I or II according to claims 1 to 3 for the manufacture of a pharmaceutical composition for the treatment or prevention of hyperlipidemia.

A further aspect of the invention is the use of a compound of formula I or II according to claims 1 to 3 for the manufacture of a pharmaceutical composition for the treatment or prevention of diabetic dyslipidemia.

A further aspect of the invention is the use of a compound of formula I or II according to claims 1 to 3 for the manufacture of a pharmaceutical composition for the treatment or prevention of the metabolic syndrome.

A further aspect of the invention is the use of a compound of formula I or II according to claims 1 to 3 for the manufacture of a pharmaceutical composition for the treatment or prevention of obesity.

A further aspect of the invention is the use of a compound of formula I or II according to claims 1 to 3 for the manufacture of a pharmaceutical composition for the treatment or prevention of diseases related to metabolic dysfunction.

### Description of Compounds of Formula I and II

The present invention is generally directed to substituted octahydroquinolizine compounds, pharmaceutical compositions containing these compounds, and methods of their pharmaceutical use.

As used herein, the terms “stereoisomers” mean compounds that possess identical chemical constitution, but differ as in regard to the arrangement of the atoms or groups in space. As used herein, the term “partial stereoisomers” mean stereoisomers having two or more chiral centers wherein at least one of the chiral centers possesses defined stereochemistry. If not indicated otherwise, stereoisomeric mixtures may contain stereoisomers and/or partial stereoisomers in different relative quantitities, which may be racemic or optically enriched themselves.

Pharmaceutically acceptable salts include, but are not limited to, the conventional salts or the quaternary ammonium salts of the parent compound formed, e.g., from non-toxic inorganic or organic acids, which are for example, prepared by methods known in the art from common inorganic acids (e.g. hydrochloric, hydrobromic, phosphoric, nitric, sulfamic, sulphuric) or the salts prepared from organic acids (e.g. ascorbic, tartaric, citric, maleic, hydroxymaleic, fumaric, oxalic, acetic, propionic, succinic, toluenesulfonic, methanesulfonic, ethane disulfonic, stearic, palmeic, glycolic, lactic, malic, phenylacetic, sulfanilic, glutamic, benzoic, salicylic, 2-acetoxybenzoic, isothioilic).

The term “effective amount” means any amount of a product as described herein that may be therapeutically effective to prevent or treat the symptoms of a particular disease, disorder, or side effect which include, but are not limited to those pathological conditions associated with diabetes.

As used herein, the term “pharmaceutically acceptable” refers to those compounds, compositions, materials, and/or dosage forms that are, within the scope of sound medical judgment, suitable for contact with the tissues of animals and human beings without excessive irritation, toxicity, allergic response, or other complications or problems commensurate with a reasonable risk-benefit ratio. The term specifically encompasses veterinary uses.

As used herein, the term “dosage unit” means physically discrete units suited as unitary dosages for the treatment of the particular individual. Each unit may contain a predetermined quantity of active material as claimed in the invention calculated to produce the desired therapeutic effect(s) in association with an adequate pharmaceutical carrier. Dosage unit forms of the invention may be dictated by the particular therapeutic effect(s) to be achieved, the unique characteristics of the active compound(s), and the limitations inherent in the art of compounding such active compound(s).

The term “treatment” or “treating” as used herein includes, without limitation, preventive (e.g., prophylactic), curative or palliative treatment. As used herein, the term “patient” means animals, including mammals, preferably humans.

Although the compounds of the present invention may be administered as pure chemicals, it is preferable to provide the active ingredient as a pharmaceutical composition, comprising an effective amount of one or more of the compounds of the invention, preferably one or more compounds of formula I or II, as described herein, together with one or more pharmaceutically acceptable carriers, i.e. in the sense of being compatible with the other ingredients of the composition and not deleterious to the recipient thereof.

The compounds of the invention may be administered in an effective amount by any of the standard techniques well-established in the medical field. The compounds employed in the methods of the present invention may be given by any means that results in the contact of the active ingredient(s) with the relevant site(s) of action in the body of a patient. The compounds may be administered by any standard means available for use in conjunction with pharmaceuticals, either as individual therapeutic agents or in a combination of therapeutic and/or preventive agents. For example, they may be administered as the sole active ingredients in a pharmaceutical composition, or they can be used in combination with other therapeutically active ingredients or with a pharmaceutically acceptable carrier in quantities, which may be determined by the solubility, the chemical nature, the route of administration or other means being advantageous for an effective treatment by the targeted therapy.

The dosage of the compounds of this invention that will be most suitable for prevention or treatment will vary with the particular compound used, the form of administration, and the physiological characteristics of the particular patient under treatment. Generally, small dosages may be used initially and, if required, increased by small increments until the desired effect under the respective circumstances is reached. Generally speaking, oral administration may require higher dosages than parenteral administrations. Although the proper dosage of the products of this invention will be readily ascertainable by one skilled in the art, once armed with the present disclosure, by way of general guidance, for example, typically a daily dosage of the compound of the invention, i.e., a compound of formula I or II, as described herein, may range from about 0.001 to about 500 milligrams per kilogram of patient body weight (and all combinations and sub-combinations of ranges and specific dosage amounts therein). Preferably, the daily dosage may be about 0.01 to about 250 milligrams per kilogram of patient body weight of the compound of this invention, preferably a compound of formula I or II.

### Methods of Preparation

If not stated otherwise, the following materials and solvents have been used: HPLC: Acetonitril (ACN) LC-MS grade (Fisher Scientific or Fluka); Water, LC-MS grade (Fisher Scientific or Fluka); Formic acid, puriss. p.a. (eluent additive for LC-MS, Fluka); Dry solvents for chemical reactions: N,N-dimethylformamide (DMF), puriss., absolute, over molecular sieve H₂O ≦0.01%, ≧99.8% (GC) (Fluka); diethyl ether (DEE), puriss., dried over molecular sieve H₂O ≦0.005%, ≧99.8% (GC) (Fluka); Tetrahydrofurane (THF), puriss., absolute, over molecular sieve H₂O ≦0.005%, ≧99.5% (GC) (Fluka); 1,2-Dimethoxyethane (DME), puriss., dried over molecular sieve (Fluka); Dichloromethane (DCM), puriss., dried over molecular sieve H₂O ≦0.005% (Fluka); Methanol (MeOH), puriss., dried over molecular sieve H₂O ≦0.01% (Fluka); Acetonitrile, puriss., dried over molecular sieve H₂O ≦0.01% (Fluka); Ethyl acetate, puriss., dried over molecular sieve H₂O ≦0.005% (Fluka)

If not stated otherwise, the following materials and solvents have been used for extraction and/or column chromatography: Petrol ether (PE): bp: 40-60° C., Baker reinst (Baker); Ethyl acetate (EtOAc), Methanol (MeOH), Diethyl ether (Et₂O): GPR Rectapur (VWR Prolabo); Cyclohexane (CyclH): Normapur (VWR Prolabo); Dichloromethane (CH₂Cl₂): for synthesis (Merck Darmstadt) or GPR Rectapur (VWR Prolabo); Toluene (Tol): Baker analyzed (Baker) or Normapur (VWR Prolabo); Ethanol (EtOH): absolute, 99.9% (Australco)

If not stated otherwise, the following reagents have been used for chemical reactions: 3-Chloropropionaldehyde diethylacetal, tech., 290% (GC) (Fluka); 1-Methylbenzyl cyanide, 96% (Aldrich); Sodium hydride (NaH), 60% dispersion in mineral oil (Aldrich); Lithiumaluminiumhydride (LAH), reagent grade, 95%, powder (Aldrich); Sulfuric acid (H₂SO₄), 95-97%, Baker analyzed (Baker; was diluted with tab-water to used concentration); Sodium hydroxide (NaOH), Baker analyzed (Baker); 3-Buten-2-one (methyl vinyl ketone), 99% (Aldrich); Hydrochloric acid (HCl), 37-38%, Baker analyzed (Baker; was diluted with tab-water to used concentration); Potassium carbonate anhydrous, purum p.a., ≧99.0% (Fluka); Sodium hydrogen carbonate (NaHCO₃) (Fluka); Potassium sodium tartrate tetrahydrate, purum p.a. ≧99.8% (Fluka); Methyllithium (MeLi) solution, purum, ˜1 M in cumene/THF (Aldrich); Jodmethane (MeI), purum: ≧99.0% (GC) (Fluka), Benzyl chloride (BnCl), puriss., ≧99.5% (GC) (Fluka); Sodium sulfate (Na₂SO₄), p.a., ACS, ISO, anhydrous (Roth); Magnesium sulfate anhydrous (MgSO₄), puriss. p.a., drying agent, ≧98% (KT) (Fluka); Ammonium chloride (NH₄Cl), purum p.a., ≧99% (Fluka); Sodium carbonate (NaCO₃), purum, ≧98.0% (T) (Fluka); Ninhydrin, 97% (Aldrich); 2-Methyl-2-phenylpropylmagnesium chloride solution 0.5 M in diethyl ether (Aldrich); Isobutylmagnesium chloride solution 2.0 M in diethyl ether (Aldrich); (1,3-Dioxan-2-ylethyl)magnesium bromide solution 0.5 M in tetrahydrofuran (Aldrich); 2-Methoxyethoxymethyl chloride (MEMCl), technical grade (Aldrich); Triethylamine (TEA), puriss. p.a., ≧99.5% (GC) (Aldrich); Diphenylacetonitrile, 98% (Aldrich); Piperidine, puriss., p.a., ≧99% (GC/T) (Fluka); Sodium cyanoborohydride, purum, ≧95% (RT) (Fluka); Ammonium hydroxide solution, purum, ˜28% in Water (Fluka); Ethylene glycol, anhydrous, 99.8% (Aldrich); 1-Bromo-2-(2-methoxyethoxy)ethane, 95% (Aldrich); p-Toluenesulfonyl-methyl isocyanide, purum, ≧98% (HPLC) (Fluka); (2-Bromoethyl)methyl ether, 95% (Aldrich); 4-Fluorophenylacetonitrile, 99% (Aldrich); 3-Amino-5-Methyl-4H-1,2,4-triazole (Fluorochem); 4-Chloroacetophenone, 97% (Aldrich); 3-Amino-1,2,4-triazole, purum, ≧95% (NT) (Fluka); Sodium borohydride, purum p.a., ≧96% (gas-volumetric) (Fluka); tert-butyllithium, 1.7 M solution in pentane (Aldrich); Lithium diisopropylamide, 1.8 M solution in THF/Heptane/Ethylbenzene (Aldrich); 3-Amino-5-trifluoromethyl-1,2,4-triazole, (UkrOrgSynthesis Ltd.); 2,2-Dimethyl-1,3-propanediol, purum, ≧98% (GC) (Fluka); Triethyl orthoformate, purum, ≧98% (GC) (Fluka); p-Toluenesulfonic acid monohydrate (Aldrich); Potassium tert-butoxide, reagent grade, 95% (Aldrich); Acetic acid glacial, 99-100%, Baker analyzed (Baker); Pyridinium chlorochromate (PCC), 98% (Aldrich); Phenethylmagnesium chloride, 1.0 M solution in THF (Aldrich); Cyclohexylmagnesium chloride, 2.0 M solution in diethyl ether (Aldrich); 2-Bromopropane, purum, ≧99% (GC) (Fluka); Acetyl chloride, puriss. p.a. ≧99% (T) (Fluka); Acetic anhydride, puriss. p.a. ≧99% (NT) (Fluka); Morpholine, puriss. p.a. ≧99% (GC) (Fluka); Benzylmagnesium chloride, 2.0 M solution in THF (Aldrich); 1-Methylpiperazine purum (GC) (Fluka); 5-Aminotetrazole, 97% (Aldrich); Sodium chloride (NaCl), purum p.a. ≧99.5% (AT) (Fluka); Dicinnamalacetone, 98% (Aldrich); Diethylamine, ≧99.5% (Aldrich); 2-Aminopyridine, purum, ≧98% (NT) (Fluka); 1-Pyrrolidinecarbonyl chloride, 97% (Aldrich); Pyrrolidine, purum, ≧98% (GC) (Fluka); 1-Bromo-2-cyclohexylethane, 98% (Aldrich); Cyclopentyl bromide, 99% (GC) (Aldrich); Dimethylcarbamyl chloride, 98% (GC, T) (Fluka); Acetone, Baker analyzed (Baker); N-Methylaniline, purum, >98% (GC) (Fluka); Cyclohexanemethylamine, 98% (Aldrich); Hydrogen, 5.0 (Messer Schweiz AG); Methylmagnesium bromide, 3.0 M solution in diethyl ether (Aldrich); Diisobutylaluminium hydride, 1.0 M solution in dichloromethane (Aldrich); Chlorotrimethylsilane, puriss.; ≧99% (Fluka); Magnesium, purum for Grignard reactions (Fluka); Cyclopentyl bromide 99% (Aldrich); 2-Aminopyrimidine 97% (Aldrich); 2-Aminobenzimidazole, technical, ≧97% (Fluka); Cyclopentylamine 99% (Aldrich); 2-Aminothiazole 97% (Aldrich); 1,3-oxazol-2-amine (bionet Key Organics LTd.); 2,5-Dimethylpyrrole 98% (Aldrich); Palladium on activated charcoal (Pd/C), puriss.; 10% (Pd) (Fluka); Boron tribromide solution, purum, ˜1 M in dichloromethane (Fluka); O-(Benzotriazol-1-yl)-N,N,N′,N′-tetramethyluronium tetrafluoroborate (TBTU) 97% (Aldrich); Potassium hydroxide (KOH), purum p.a., ≧85%, pellets (Fluka); Paraformaldehyde, purum ≧95% (Fluka); Triphenylphosphine, purum ≧95% (Sigma-Aldrich); Diethyl azodicarboxylate solution (DEAD), purum ˜40% in toluene (Aldrich)

If not stated otherwise, reaction mixtures are purified by Column Chromatography (CC) on Silica gel (silica gel 60, 0.06-0.2 mm, Roth or Merck; Davisil LC60A 60-200μ, Grace Davison) following common procedures. In CC method A a gradient of CH₂Cl₂/MeOH is used, in CC method B a gradient of PE/EtOAc is used, in CC method C a gradient of PE/EtOAc with 1% TEA, in CC method D a gradient of CyclH/EtOAc with 1% TEA, in CC method E a gradient of CH₂Cl₂/MeOH with 0.1% NH₄OH (28% aq. solution), in CC method F a gradient of CyclH/EtOAc is used, in CC method G a gradient of Tol/EtOAc with 1% TEA. Eluates are examined by Thin Layer Chromatography (TLC), unified as single spot products or defined stereoisomeric mixtures and evaporated to dryness under reduced pressure (bath temperature 20-40° C.); TLC plates: TLC silica gel 60 F₂₅₄ glass plates 20*20 cm Multiformat pre-scored to 5*10 cm (Merck) or GraceResolv silica TLC plates, hard layer with organic binder, 254 nm fluorescent indicator, 20*20 cm scored plates (Grace Davison); detection by UV and/or staining with Ninhydrin solution (0.2 g in 100 ml ethanol, heat).

If not stated otherwise, the reaction products are identified and/or characterized by HPLC/MS or MS direct infusion. HPLC/MS: Instrumentation: SCL-10AVP, controller; DGU-20A5, degasser, FCV-10ALVP, low pressure gradient mixing unit, LC-10ADVP pump, SIL10AP, autosampler with 500 μl syringe and 400 μl injection loop, SPD-M10AVP, PDA detector, LCMS 2010A MS detector (Shimadzu); SmartMix, gradient mixer with 350 μl mixing chamber (Knauer); N₂ LCMS 1, nitrogen generator (Claind); E2M28, two stage rotary vacuum pump (Edwards); Software: LabSolutions—LCMSolution Ver. 3.41 (Shimadzu); Sample preparation: Samples are weighted, dissolved in acetonitril, and diluted to a final volume of 1 ml with a concentration of 0.5-0.05 mg/ml in acetonitril/water (with 0.1% formic acid)=9:1. The injection volume was adjusted (1-10 μl) to achieve an injection of 0.5 μg sample. Solvents: solvent A: water with 0.1% formic acid, solvent B: acetonitril with 0.1% formic acid. MS direct infusion: Instrumentation: LCMS 2010A MS detector (Shimadzu); N₂ LCMS 1, nitrogen generator (Claind); E2M28, two stage rotary vacuum pump (Edwards); Software: LabSolutions—LCMSolution Ver. 3.41 (Shimadzu); Sample preparation: Samples are weighted, dissolved in acetonitril/water containing 0.1% formic acid and diluted to a concentration of 1 ppm. Samples are continuously injected into MS (direct infusion mode).

Reaction products and stereoisomers are characterised by HPLC/MS via relative retention time in minutes after injection (RTT) or by MS direct infusion applying the following methods as mentioned in the examples below. Detected ions are given in intensities in percent relative to base peak (100%). HPLC/MS Method A: Column: Synergi 4μ Fusion-RP 80A 150×2.0 mm, with Security Guard Cartridge Fusion-RP 4×2.0 mm (Phenomenex Inc.); flow: 0.5 ml/min; linear gradient (% A is the difference to 100%): start at 10% B, in 10 min to 50% B, then in 2 min to 100% B, then kept for 10 min at 100% B, then in 3 min to 10% B, then 10 min equilibration at 10% B; total run time: 35 min; PDA detector: wavelength: 190-600 nm, sampling rate: 1.56 Hz, MS detector: ionization mode: ESI positive, mass range: 150-500±0.5 m/z; scan speed: 500 amu/sec; detector voltage: 1.25 kV; heat block temperature: 200° C.; CDL temperature: 250° C.; nebulizing gas flow: 1.5 L/min; dry gas pressure: 0.1 MPa. Method B: Column: Synergi 4μ Polar-RP 80A 150×2.0 mm, with Security Guard Cartridge Polar-RP 4×2.0 mm (Phenomenex Inc.); flow: 0.5 ml/min; linear gradient (% A is the difference to 100%): start at 10% B, in 10 min to 50% B, then in 2 min to 100% B, then kept for 10 min at 100% B, then in 3 min to 10% B, then 10 min equilibration at 10% B; total run time: 35 min; PDA detector: wavelength: 190-600 nm, sampling rate: 1.56 Hz, MS detector: ionization mode: ESI positive, mass range: 100 or 150-500 or 600±0.5 m/z; scan speed: 500 amu/sec; detector voltage: 1.25 kV; heat block temperature: 200° C.; CDL temperature: 250° C.; nebulizing gas flow: 1.5 L/min; dry gas pressure: 0.1 MPa. MS direct infusion: MS detector: continuous injection with 10 μl/min, ionization mode: ESI positive, mass range: 150-700±0.5 m/z; scan speed: 500 amu/sec; detector voltage: 1.3-1.5 kV; heat block temperature: 200° C.; CDL temperature: 250° C.; nebulizing gas flow: 1.5 L/min; dry gas pressure: 0.1 MPa.

If not stated otherwise, RT stands for room temperature or ambient temperature, which typically lies between 20 and 25° C.

### EXAMPLES

### Example 1

### Preparation of Products with Product No. 1 and 2 Following the Procedure of Frank D. King, J. Chem. Soc. Perkin Trans. 1, 447-453 (1986)

To a suspension of dry DMF (100 ml) and sodium hydride, 60% (4.1 g, 0.17 mol) at 70° C. in a dry and inert atmosphere (Argon) methylphenylacetonitrile (10 ml, 0.075 mol) is added. After stirring at 70° C. for 1 h 3-chloropropionaldehyde diethyl acetal (13.2 g, 0.079 mol) is added drop wise. After stirring at 70° C. for 1 h and cooling to RT, the reaction mixture is poured into 1 L of ice-water. The product is extracted with Et₂O (3×200 ml). The combined Et₂O extracts are filtered over Na₂SO₄ and evaporated in vac. to dryness yielding about 19.1 g of crude product which is directly used in the next step. To a suspension of dry THF (115 ml) and LAH (2.43 g, 0.065 mol) in a dry and inert atmosphere (Argon) concentrated sulphuric acid (1.6 ml, 0.03 mol) is added drop wise under cooling with ice-water. After stirring at 0° C. for 1 h, a solution of the crude product from the previous step (19.1 g, 0.073 mol) in dry THF (19 ml) is added drop wise and the reaction mixture is stirred at RT for 5 h. After cooling to 0° C. 1 M NaOH (11.3 ml) is added, the formed precipitate is removed by suction filtration and Et₂O is used to wash the precipitate. The filtrate is evaporated in vac. to dryness yielding about 16 g crude product, which is dissolved in 65 ml Et₂O. Methylvinylketone (6.1 ml, 0.073 mol) is added and the reaction mixture is stirred at RT for 2 h. The reaction mixture is added drop wise to 350 ml of 2.5 M HCl. The phases are separated with a separator funnel, the aqueous phase is taken and refluxed for 2 h. After addition of ice cubes, the mixture is neutralized with solid sodium carbonate and extracted with CH₂Cl₂. The combined organic layers are dried over Na₂SO₄ and the solvent is removed in vac. to dryness. The crude product (11.2 g) is purified by CC method B yielding 1 (2.9 g) and 2 (5.1 g).

HPLC/MS Method A: 1: RTT=2.7 [ms: 262.1 (M+H₃O⁺), 244.1 (35%, M+H⁺)]; 2: RTT=3.6 [ms: 262.1 (M+H₃O⁺), 244.1 (25%, M+H⁺)]

### Example 2

### Preparation of Products 3, 4, 302, 303, 312 and 313

Product 1, 2, 149, 150, 296 or 297, respectively, dissolved in dry DEE is added slowly to a stirred suspension of 1 eq. LAH in dry DEE in a dry and inert atmosphere (Argon) at RT. After 2 h the reaction mixture is quenched with water, rendered alkaline with NaOH and extracted three times with Et₂O. The combined organic layers are dried over Na₂SO₄, filtered and evaporated in vac. to dryness yielding 3, 4, 302, 303, 312 or 313, respectively.

HPLC/MS Method A: 3: Stereoisomer I: RTT=2.9 [ms: 246.1 (M+H⁺)], Stereoisomer II: RTT=3.4 [ms: 246.1 (M+H⁺)]; 4: Stereoisomer I: RTT=3.9 [ms: 246.1 (M+H⁺)], Stereoisomer II: RTT=4.6 [ms: 246.1 (M+H⁺)]; HPLC/MS Method B: 303: Stereoisomer I: RTT=8.9 [ms: 260.2 (M+H⁺)], Stereoisomer II: RTT=9.1 [ms: 260.2 (M+H⁺)]; 313: RTT=10.7 [ms: 302.3 (M+H⁺)]

### Example 3

### Preparation of Products 5, 6, 17 to 20, 45 to 52

Product 3, 4, 9 to 12, 27 to 30, 39 to 42, respectively, is dissolved in dry DMF, 2 eq. NaH are added and the reaction mixture is stirred at RT for 45 min. 1.2 to 5 eq. MeI are added and the reaction mixture is stirred over night. The reaction mixture is quenched with water and extracted with CH₂Cl₂. The combined organic phases are dried over MgSO₄, filtered and evaporated in vac. to dryness yielding 5, 6, 17 to 20, 45 to 52, respectively. Product 5, 6, 17 to 20, respectively, is purified by CC method B, product 47 and 49, respectively, is purified by CC method D.

HPLC/MS Method A: 5: Stereoisomer I: RTT=4.6 [ms: 260.1 (M+H⁺)], Stereoisomer II: RTT=6.2 [ms: 260.1 (M+H⁺)]; 6: Stereoisomer I: RTT=7.1 [ms: 260.1 (M+H⁺)], Stereoisomer II: RTT=8.4 [ms: 260.1 (M+H⁺)]; 17: RTT=6.0 [ms: 274.1 (M+H⁺), 242.1 (4%, M+H⁺-MeOH)]; 18: RTT=7.7 [ms: 274.2 (M+H⁺)]; 19: RTT=7.3 [ms: 274.1 (M+H⁺), 242.2 (8%, M+H⁺-MeOH)]; 20: RTT=8.9 [ms: 274.1 (M+H⁺)]; 49: RTT=10.9 [ms: 316.2 (M+H⁺)];

HPLC/MS Method B: 47: RTT=12.2 [ms: 374.3 (M+H⁺)]

### Example 4

### Preparation of Products 7, 8, 21 to 24

Product 3, 4, 9 to 12 respectively, is dissolved in dry DMF, 2 eq. NaH are added and the reaction mixture is stirred at RT for 45 min. 1.2 to 5 eq. BnCl are added and the reaction mixture is stirred over night. The reaction mixture is quenched with water and extracted with Et₂O. The combined organic phases are dried over MgSO₄, filtered and evaporated in vac. to dryness yielding 7, 8, 21 to 24. Product 7, 8, 21 to 24, respectively is purified by CC method B.

HPLC/MS Method A: 7: Stereoisomer I: RTT=10.7 [ms: 336.1 (M+H⁺)], Stereoisomer II: RTT=11.4 [ms: 336.1 (M+H⁺)]; 8: Stereoisomer I: RTT=11.4 [ms: 336.1 (M+H⁺)], Stereoisomer II: RTT=12.2 [ms: 336.1 (M+H⁺)]; 21: RTT=11.3 [ms: 350.2 (M+H⁺), 242.1 (4%, M+H⁺-BnOH)]; 22: RTT=11.8 [ms: 350.2 (M+H⁺)]; 23: RTT=11.6 [ms: 350.1 (M+H⁺), 242.1 (3%, M+H⁺-BnOH)]; 24: RTT=12.3 [ms: 350.1 (M+H⁺)]

### Example 5

### Preparation of Products 9 to 12, 198, 202 and 203

Product 1, 2, 197, 149 or 150, respectively, is dissolved in a dry inert atmosphere (Argon) in dry DEE and cooled to −70° C. 2 eq. MeLi are added and kept for 1 h at −70° C. without stirring. After warming to RT over a period of 1 h the reaction mixture is hydrolyzed with aq. NH₄Cl solution and extracted with Et₂O. The organic phase is dried over MgSO₄, filtered and evaporated in vac. to dryness yielding 9 and 10, 11 and 12, 198, 202 or 203, respectively. Product 9 to 12, 198, 203, respectively, is purified by CC method A.

HPLC/MS Method A: 9: RTT=3.3 [ms: 260.1 (M+H⁺), 242.1 (4%, M+H⁺—H₂O)]; 10: RTT=4.5 [ms: 260.1 (M+H⁺)]; 11: RTT=5.1 [ms: 260.1 (M+H⁺), 242.1 (2%, M+H⁺—H₂O)]; 12: RTT=5.6 [ms: 260.1 (M+H⁺)]; 203: RTT=6.5 [ms: 274.2 (M+H⁺)]; MS direct infusion: 198: [ms: 322.3 (M+H⁺)]

### Example 6

### Preparation of Products 13 to 16, 177 and 178

Product 1 or 2, 149 or 150, respectively, is dissolved in dry DEE, 2 eq. benzylmagnesium chloride solution is added and stirred at RT for 1.5 h. The reaction mixture is quenched with NH₄Cl solution and extracted with CH₂Cl₂. The combined organic phases are dried over MgSO₄, filtered and evaporated in vac. to dryness yielding 13 and 14, 15 and 16, 177 or 178, respectively. Product 13 to 16 and 178 are purified by CC method D.

HPLC/MS Method A: 13: RTT=10.1 [ms: 336.3 (M+H⁺)]; 14: RTT=9.7 [ms: 336.3 (M+H⁺), 318.3 (8%, M+H⁺—H₂O)]; 15: RTT=10.2 [ms: 336.3 (M+H⁺)]; 16: RTT=10.6 [ms: 336.3 (M+H⁺), 318.3 (8%, M+H⁺—H₂O)]; HPLC/MS Method B: 178: RTT=13.0 [ms: 350.3 (M+H⁺)]

### Example 7

### Preparation of Products 25 and 26

Product 3 or 4, respectively, is dissolved in dry DMF, 2 eq. NaH are added and the reaction mixture is stirred at RT for 45 min. 1.2 to 5 eq. MEMCl are added and the reaction mixture is stirred over night. The reaction mixture is quenched with water and extracted with CH₂Cl₂. The combined organic phases are dried over MgSO₄, filtered and evaporated in vac. to dryness yielding 25 or 26 respectively. Product 25 and 26 are purified by CC method C.

HPLC/MS Method A: 25: Stereoisomer I: RTT=7.8 [ms: 334.2 (M+H⁺)], Stereoisomer II: RTT=8.5 [ms: 334.1 (M+H⁺)]; 26: Stereoisomer I: RTT=8.7 [ms: 334.1 (M+H⁺)], Stereoisomer II: RTT=9.5 [ms: 334.1 (M+H⁺)]

### Example 8

### Preparation of Products 27 to 30, 155, 156, 183, 184, 189 and 190

Product 1 or 2, 149 to 154, respectively, is dissolved in dry DEE, 1.2 eq. (1,3-dioxan-2-ylethyl)magnesium bromide solution are added and the reaction mixture is stirred at RT for 1.5 h. The reaction mixture is quenched with NH₄Cl solution and extracted with Et₂O. The combined organic phases are dried over MgSO₄, filtered and evaporated in vac. to dryness yielding 27 and 28, 29 and 30, 155, 156, 183, 184, 189 and 190. Product 27 to 30 is purified by CC method B, product 155, 156, 184 and 190 is purified by CC method D.

HPLC/MS Method A: 27: RTT=8.3 [ms: 360.1 (M+H⁺)]; 28: RTT=7.8 [ms: 360.1 (M+H⁺), 342.2 (3%, M+H⁺—H₂O)]; 30: RTT=9.0 [ms: 360.2 (M+H⁺), 342.2 (4%, M+H⁺—H₂O)]; 155: RTT=9.0 [ms: 374.1 (M+H⁺)]; 156: RTT=9.0 [ms: 374.1 (M+H⁺)]; HPLC/MS Method B: 29: RTT=10.6 [ms: 360.3 (M+H⁺)]; 184: RTT=12.2 [ms: 408.3, 410.2 (37%) (M+H⁺)]; 190: RTT=11.3 [ms: 392.3 (M+H⁺)]

### Example 9

### Preparation of Products 31 to 34

Product 1 or 2, respectively, is dissolved in dry DEE, 1.2 eq. 4-methylbenzylmagesium chloride solution is added and the reaction mixture is stirred at RT for 1.5 h. The reaction mixture is quenched with NH₄Cl solution and extracted with Et₂O. The combined organic phases are dried over MgSO₄, filtered and evaporated in vac. to dryness yielding 31 and 32 or 33 and 34, respectively.

HPLC/MS Method A: 31: RTT=11.0 [ms: 350.3 (M+H⁺)]; 32: RTT=10.6 [ms: 350.3 (M+H⁺), 332.3 (9%, M+H⁺—H₂O)]; 33: RTT=11.1 [ms: 350.3 (M+H⁺)]; 34: RTT=11.4 [ms: 350.3 (M+H⁺), 332.3 (8%, M+H⁺—H₂O)]

### Example 10

### Preparation of Products 35 to 38

Product 1 or 2, respectively, is dissolved in dry DEE, 1.2 eq. 2-methyl-2-phenylpropylmagnesium chloride solution are added and the reaction mixture is stirred at RT for 1.5 h. The reaction mixture is quenched with NH₄Cl solution and extracted with Et₂O. The combined organic phases are dried over MgSO₄, filtered and evaporated in vac. to dryness yielding 35 and 36 or 37 and 38, respectively. Product 35 to 38 is purified by CC method B.

HPLC/MS Method A: 35: RTT=11.9 [ms: 378.2 (M+H⁺)]; 36: RTT=11.8 [ms: 378.2 (M+H⁺), 360.2 (7%, M+H⁺—H₂O)]; 37: RTT=12.1 [ms: 378.2 (M+H⁺)]; 38: RTT=12.3 [ms: 378.2 (M+H⁺), 360.2 (6%, M+H⁺—H₂O)]

### Example 11

### Preparation of Products 39 to 42

Product 1 or 2, respectively, is dissolved in dry DEE, 1.2 eq. isobutylmagnesium chloride solution are added and the reaction mixture is stirred at RT for 1.5 h. The reaction mixture is quenched with NH₄Cl solution and extracted with Et₂O. The combined organic phases are dried over MgSO₄, filtered and evaporated in vac. to dryness yielding 39 and 40 or 41 and 42, respectively. Product 39 to 41 is purified by CC method B.

HPLC/MS Method A: 39: RTT=9.7 [ms: 302.2 (M+H⁺)]; 40: RTT=9.2 [ms: 302.2 (M+H⁺), 284.1 (8%, M+H⁺—H₂O)]; 41: RTT=9.9 [ms: 302.2 (M+H⁺)]; 42: RTT=10.3 [ms: 302.2 (M+H⁺), 284.2 (11%, M+H⁺—H₂O)]

### Example 12

### Preparation of Products 43 and 44

Product 3 or 4, respectively, is dissolved in dry DMF, 2 eq. NaH are added and the reaction mixture is stirred at RT for 45 min. 1.2 to 5 eq. (2-bromoethyl)methylether are added and the reaction mixture is stirred over night. The reaction mixture is quenched with water and extracted with CH₂Cl₂. The combined organic phases are dried over MgSO₄, filtered and evaporated in vac. to dryness yielding 43 or 44, respectively. Product 44 is purified by CC method D.

HPLC/MS Method A: 44: Stereoisomer I: RTT=7.3 [ms: 304.2 (M+H⁺)], Stereoisomer II: RTT=8.4 [ms: 304.2 (M+H⁺)]

### Example 13

### Preparation of Products 53 to 56

Product 1 or 2, respectively, is dissolved in a dry inert atmosphere (Argon) in dry DEE and cooled to −70° C. 2 eq. tert-BuLi are added and the reaction mixture is kept for 1 h at −70° C. without stirring. After warming to RT over a period of 1 h the reaction mixture is hydrolyzed with aq. NH₄Cl solution and extracted with Et₂O. The organic phase is dried over MgSO₄, filtered and evaporated in vac. to dryness yielding 53 and 54 or 55 and 56, respectively.

HPLC/MS Method A: 55: RTT=9.2 [ms: 302.2 (M+H⁺)]; 56: RTT=9.9 [ms: 302.2 (M+H⁺), 284.1 (4%, M+H⁺—H₂O)]

### Example 14

### Preparation of Products 57 to 60

Product 9 to 12, respectively, is dissolved in dry DMF, 3 eq. NaH are added and the reaction mixture is stirred at RT for 45 min. 5 eq. (2-bromoethyl)methylether are added and the reaction mixture is stirred over night. The reaction mixture is quenched with water and extracted with Et₂O. The combined organic phases are dried over MgSO₄, filtered and evaporated in vac. to dryness yielding 57 to 60. Product 60 is purified by CC method D.

HPLC/MS Method A: 60: RTT=9.3 [ms: 318.2 (M+H⁺)]

### Example 15

### Preparation of Products 61 to 64, 208 and 209

Product, 9 to 12, 3 or 4, respectively, is dissolved in dry DMF, 3 eq. NaH are added and the reaction mixture is stirred at RT for 45 min. 5 eq. 1-Bromo-2-(2-methoxyethoxy)ethane are added and the reaction mixture is stirred over night. The reaction mixture is quenched with water and extracted with Et₂O. The combined organic phases are dried over MgSO₄, filtered and evaporated in vac. to dryness yielding 61 to 64, 208 or 209, respectively. Product 64 and 209 is purified by CC method D.

HPLC/MS Method A: 64: RTT=9.6 [ms: 362.2 (M+H⁺)]; 209: Steroisomere I: RTT=8.6 [ms: 348.1 (M+H⁺), 370.1 (13%, (M+Na⁺)], Steroisomere II: RTT=9.2 [ms: 348.1 (M+H⁺)]

### Example 16

### Preparation of Products 65 to 68

Product 1 or 2, respectively, is dissolved in dry DEE, 1.2 eq. phenylethylmagnesium chloride solution are added and the reaction mixture is stirred at RT for 1.5 h. The reaction mixture is quenched with NH₄Cl solution and extracted with Et₂O. The combined organic phases are dried over MgSO₄, filtered and evaporated in vac. to dryness yielding 65 and 66 or 67 and 68, respectively. Product 65 and 67 is purified by CC method D.

HPLC/MS Method A: 65: RTT=11.0 [ms: 350.1 (M+H⁺)]; 66: RTT=10.8 [ms: 350.1 (M+H⁺), 332.1 (3%, M+H⁺—H₂O)]; 67: RTT=11.1 [ms: 350.1 (M+H⁺)]; 68: RTT=11.4 [ms: 350.1 (M+H⁺), 332.1 (4%, M+H⁺—H₂O)]

### Example 17

### Preparation of Products 69 to 72

Product 1 or 2, respectively, is dissolved in dry DEE, 1.2 eq. cyclohexylmagnesium chloride solution are added and the reaction mixture is stirred at RT for 1.5 h. The reaction mixture is quenched with NH₄Cl solution and extracted with Et₂O. The combined organic phases are dried over MgSO₄, filtered and evaporated in vac. to dryness yielding 69 and 70 or 71 and 72, respectively. Product 71 is purified by CC method D.

HPLC/MS Method A: 69: RTT=10.6 [ms: 328.2 (M+H⁺)]; 70: RTT=10.1 [ms: 328.2 (M+H⁺), 310.2 (12%, M+H⁺—H₂O)]; 71: RTT=10.5 [ms: 328.2 (M+H⁺)]; 72: RTT=10.9 [ms: 328.2 (M+H⁺), 310.2 (12%, M+H⁺—H₂O)]

### Example 18

### Preparation of Products 73 to 76

2 eq. cyclohexylethyl bromide are dissolved in a dry inert atmosphere (Argon) in dry THF, 2 eq. tert-BuLi are added and kept for 30 min at −70° C. without stirring. Product 1 or 2, respectively, dissolved in dry THF is added and kept for 1 h at −70° C. without stirring. After warming to RT over a period of 1 h the reaction mixture is hydrolyzed with aq. NH₄Cl solution and extracted with Et₂O. The organic phase is dried over MgSO₄, filtered and evaporated in vac. to dryness yielding 73 and 74 or 75 and 76, respectively. Product 75 is purified by CC method D.

HPLC/MS Method A: 76: RTT=12.6 [ms: 356.2 (M+H⁺), 338.2 (3%, M+H⁺—H₂O)];

HPLC/MS Method B: 73: RTT=15.0 [ms: 356.3 (M+H⁺)]; 74: RTT=14.6 [ms: 356.3 (M+H⁺), 338.3 (7%, M+H⁺—H₂O)]; 75: RTT=14.8 [ms: 356.3 (M+H⁺)]

### Example 19

### Preparation of Products 77 to 80, 125 to 128, 157, 158, 185, 186, 191, 192

Product 27 to 30, 45 to 48, 155, 156, 183, 184, 189 or 190, respectively, is dissolved in a 5% aqueous HCl solution and stirred over night at RT. The reaction mixture is diluted with water, rendered alkaline with solid sodium carbonate (pH 11) and extracted with CH₂Cl₂. The organic phase is dried over MgSO₄, filtered and evaporated in vac. to dryness yielding 77 to 80, 125 to 128, 157, 158, 185, 186, 191 or 192 respectively.

HPLC/MS Method B: 79: RTT=10.1 [ms: 302.3 (M+H⁺)]; 127: RTT=10.8 [ms: 316.2 (M+H⁺), 334.3 (26%, M+H₃O⁺)]; 158: RTT=10.6 [ms: 316.3 (M+H⁺)]; 192: RTT=10.9 [ms: 334.3 (M+H⁺)]

### Example 20

### Preparation of Products 81 to 84, 159, 160, 187, 188

Product 77 to 80, 157, 158, 185 or 186, respectively, is dissolved in methanol, 2 eq. morpholine and 2 eq. acetic acid are added. After 1 h 4 eq. sodium cyanoborohydride are added and the reaction mixture is stirred at RT over night. The reaction mixture is poured into a saturated sodium bicarbonate solution and extracted with CH₂Cl₂. The organic phase is dried over MgSO₄, filtered and evaporated in vac. to dryness yielding 81 to 84, 159, 160, 187 or 188, respectively. Product 81 to 84, 159, 160 and 188 is purified by CC method E. delivered 81 to 84, 159, 160, 187 or 188 as pure products.

HPLC/MS Method A: 83: RTT=5.1 [ms: 187.3 (M+2H⁺), 207.7 (17%, M+ACN+2H⁺), 373.2 (69%, M+H⁺)]; HPLC/MS Method B: 81: RTT=3.8 [ms: 187.2 (M+2H⁺), 207.9 (53%, M+ACN+2H⁺), 373.3 (93%, M+H⁺)]; 82: RTT=3.0 [ms: 187.3 (M+2H⁺), 207.7 (36%, M+ACN+2H⁺), 373.3 (73%, M+H⁺)]; 84: RTT=4.1 [ms: 187.2 (94%, M+2H⁺), 207.7 (56%, M+ACN+2H⁺), 373.3 (M+H⁺)]; 159: RTT=4.4 [ms: 194.3 (M+2H⁺), 214.8 (38%, M+ACN+2H⁺), 387.4 (66%, M+H⁺)]; 160: RTT=5.3 [ms: 194.3 (38%, M+2H⁺), 214.8 (39%, M+ACN+2H⁺), 387.3 (M+H⁺)]; 188: RTT=8.7 [ms: 211.3 (59%, M+2H⁺); 231.8 (63%, M+ACN+2H⁺); 421.4, 423.4 (34%) (M+H⁺)]

### Example 21

### Preparation of Products 85 to 88

Product 77 to 80, respectively, is dissolved in methanol, 2 eq. piperidine and 2 eq. acetic acid are added. After 1 h 4 eq. sodium cyanoborohydride are added and the reaction mixture is stirred at RT over night. The reaction mixture is poured into a saturated sodium bicarbonate solution and extracted with CH₂Cl₂. The organic phase is dried over MgSO₄, filtered and evaporated in vac. to dryness yielding 85 to 88, respectively. Product 87 is purified by CC method E.

HPLC/MS Method B: 87: RTT=7.1 [ms: 186.2 (M+2H⁺), 206.9 (31%, M+ACN+2H⁺), 371.4 (94%, M+H⁺)]

### Example 22

### Preparation of Products 89 to 92

Product 77 to 80 respectively, is dissolved in methanol, 2 eq. diethylamine and 2 eq. acetic acid are added. After 1 h 4 eq. sodium cyanoborohydride are added and the reaction mixture is stirred at RT over night. The reaction mixture is poured into a saturated sodium bicarbonate solution and extracted with CH₂Cl₂. The organic phase is dried over MgSO₄, filtered and evaporated in vac. to dryness yielding 89 to 92 respectively. Product 91 is purified by CC method E.

HPLC/MS Method B: 91: RTT=6.1 [ms: 180.2 (M+2H⁺), 200.8 (24%, M+ACN+2H⁺), 359.3 (60%, M+H⁺)]

### Example 23

### Preparation of Products 93 to 96, 161 and 162

Product 77 to 80, 157 or 158, respectively, is dissolved in methanol, 1.3 eq. N-methylaniline and 1.3 eq. acetic acid are added. After 1 h 4 eq. sodium cyanoborohydride are added and the reaction mixture is stirred at RT over night. The reaction mixture is poured into a saturated sodium bicarbonate solution and extracted with CH₂Cl₂. The organic phase is dried over MgSO₄, filtered and evaporated in vac. to dryness yielding 93 to 96, 161 or 162 respectively. Product 95 and 162 is purified by CC method E.

HPLC/MS Method A: 95: RTT=7.7 [ms: 197.3 (M+2H⁺), 217.8 (26%, M+ACN+2H⁺), 393.2 (68%, M+H⁺)]; HPLC/MS Method B: 162: RTT=10.0 [ms: 204.3 (M+2H⁺), 224.8 (66%, M+ACN+2H⁺), 407.3 (99%, M+H⁺)]

### Example 24

### Preparation of Products 97 to 100

Product 1 or 2, respectively, is dissolved in methanol, 1.3 eq. cyclohexylmethylamine and 1.3 eq. acetic acid are added. After 1 h 3 eq. sodium cyanoborohydride are added and the reaction mixture is stirred at RT over night. The reaction mixture is poured into a saturated sodium bicarbonate solution and extracted with CH₂Cl₂. The organic phase is dried over MgSO₄, filtered and evaporated in vac. to dryness yielding 97 and 98 or 99 and 100, respectively. Product 99 and 100 is purified by CC method D.

HPLC/MS Method B: 99: RTT=8.8 [ms: 171.2 (3%, M+2H⁺), 191.9 (51%, M+ACN+2H⁺), 341.3 (M+H⁺)]; 100: RTT=9.0 [ms: 171.3 (6%, M+2H⁺), 191.7 (83%, M+ACN+2H⁺), 341.3 (M+H⁺)]

### Example 25

### Preparation of Products 101 to 104

Morpholine is dissolved in CH₂Cl₂ and acetic anhydride is added drop wise under ice/water cooling. After stirring at RT over night the reaction mixture is washed once with distilled water, once with 15% HCl solution and finally with a saturated sodium carbonate solution. The organic phase is dried over MgSO₄, filtered and evaporated in vac. to dryness yielding 4-acetylmorpholine sufficiently pure for further conversion.

1.6 eq 4-acetylmorpholine is dissolved in a dry inert atmosphere (Argon) in dry THF and cooled to −78° C. 1.9 eq. LDA solution are added and kept for 20 min at −78° C. Product 1 or 2, respectively, is dissolved in dry THF and added at −78° C. to the reaction mixture. The reaction mixture is warmed to RT and kept at RT for 1 h. After cooling to −78° C. 2 eq. LAH are added, warmed to RT and stirred over night. 5 eq. of potassium sodium tartrate tetrahydrate are added, the precipitate is filtered of and the filtrate is evaporated in vac. to dryness yielding 101 and 102 or 103 and 104, respectively. Product 103 and 104 is purified by CC method E.

HPLC/MS Method B: 103: RTT=4.3 [ms: 180.2 (87%, M+2H⁺), 200.9 (53%, M+ACN+2H⁺), 359.2 (M+H⁺)]; 104: RTT=3.2 [ms: 180.3 (M+2H⁺), 200.8 (29%, M+ACN+2H⁺), 359.2 (92%, M+H⁺)]

### Example 26

### Preparation of Products 105 to 108, 248, 249

Product 77 to 80, 157 or 158, respectively, is dissolved in acetone, 10 eq. PCC are added and the reaction mixture is stirred at RT over night. After evaporation of the solvent the residue is partitioned between water and CH₂Cl₂, the organic phase is dried over MgSO₄, filtered and evaporated in vac. to dryness. The residue is redissolved in a mixture of CyclH/EtOAc (1:1) containing 1% of TEA and filtered over aluminium oxide yielding 105 to 108, 248 or 249, respectively.

HPLC/MS Method B: 107: RTT=10.6 [ms: 300.1 (M+H⁺)]; 249: RTT=10.4 [ms: 314.3 (M+H⁺)]

### Example 27

### Preparation of Products 109 to 112, 129 to 132, 242 and 243

Product 77 to 80, 125 to 128, 157 or 158, respectively, is dissolved in methanol, cooled to 0° C. and 10 eq. sodium borohydride were added. After 2 h at RT the reaction mixture is poured into a saturated sodium bicarbonate solution and extracted with CH₂Cl₂. The organic phase is dried over MgSO₄, filtered and evaporated in vac. to dryness yielding 109 to 112, 129 to 132, 242 or 243, respectively.

HPLC/MS Method B: 109: RTT=8.4 [ms: 304.2 (M+H⁺)]; 111: RTT=9.0 [ms: 304.2 (M+H⁺)]; 131: RTT=10.4 [ms: 318.2 (M+H⁺)]; 243: RTT=9.3 [ms: 318.3 (M+H⁺)]

### Example 28

### Preparation of Products 113 to 116

Product 109 to 112, respectively, is dissolved in dry DMF and 3 eq. sodium hydride are added under stirring. After 45 min 1.1 eq 1-pyrrolidinecarbonyl chloride are added and the reaction mixture is stirred for 2 h at RT. The reaction mixture is quenched with distilled water and the aqueous phase is extracted Et₂O. The organic phase is dried over MgSO₄, filtered and evaporated in vac. to dryness yielding 113 to 116, respectively. Product 113 and 115 is purified by CC method E.

HPLC/MS Method B: 113: RTT=11.9 [ms: 401.2 (M+H⁺)]; 115: RTT=12.2 [ms: 401.2 (M+H⁺)]

### Example 29

### Preparation of Products 117 to 120

Product 109 to 112, respectively, is dissolved in dry DMF and 3 eq. sodium hydride are added under stirring. After 45 min 1.1 eq N,N-dimethylcarbamyl chloride are added and the reaction mixture is stirred for 2 h at RT. The reaction mixture is quenched with distilled water and the aqueous phase is extracted Et₂O. The organic phase is dried over MgSO₄, filtered and evaporated in vac. to dryness yielding 117 to 120, respectively. Product 119 is purified by CC method E.

HPLC/MS Method B: 119: RTT=11.4 [ms: 375.2 (M+H⁺)]

### Example 30

### Preparation of Products 121 to 124, 272 and 273

Product 77 to 80, 157 or 158, respectively, is dissolved in DMF/AcOH=9:1, 10 eq. 2-aminopyridine are added and the reaction mixture is heated to 95° C. for 2 h. After cooling to RT 10 eq. sodium cyanoborohydride are added and the reaction mixture is stirred at RT over night. The reaction mixture is poured into a saturated sodium bicarbonate solution and extracted with CH₂Cl₂. The organic phase is dried over MgSO₄, filtered and evaporated in vac. to dryness. The crude product is dissolved in dry DEE, 3 eq. LAH are added and the reaction mixture is stirred for 1 h. The reation mixture is quenched with water and extracted with Et₂O. The organic phase is dried over MgSO₄, filtered and evaporated in vac. to dryness yielding 121 to 124, 272 or 273, respectively. Product 123 and 273 is purified by CC method E.

HPLC/MS Method B: 123: RTT=8.4 [ms: 190.8 (M+2H⁺), 211.4 (59%, M+ACN+2H⁺), 380.3 (40%, M+H⁺)]; 273: RTT=8.3 [ms: 197.8 (M+2H⁺), 218.2 (27%, M+ACN+2H⁺), 394.3 (82%, M+H⁺)]

### Example 31

### Preparation of Products 133 to 136

Product 125 to 128 respectively, is dissolved in methanol, 10 eq. 5-aminotetrazole and 5 eq. sodium cyanoborohydride are added and the reaction mixture is stirred at RT over night. The reaction mixture is diluted with water and extracted with CH₂Cl₂. The organic phase is dried over MgSO₄, filtered and evaporated in vac. to dryness yielding 133 to 136 respectively. Product 135 is purified by CC method E.

HPLC/MS Method B: 135: RTT=10.8 [ms: 385.3 (M+H⁺)]

### Example 32

### Preparation of Products 137 to 140

Product 125 to 128 respectively, is dissolved in methanol, 10 eq. 3-amino-1,2,4-triazole and 10 eq. acetic acid are added. After 2 h 4 eq. sodium cyanoborohydride are added and the reaction mixture is stirred at RT over night. The reaction mixture is poured into a saturated sodium bicarbonate solution and extracted with CH₂Cl₂. The organic phase is dried over MgSO₄, filtered and evaporated in vac. to dryness yielding 137 to 140, respectively. Product 139 is purified by CC method E.

HPLC/MS Method B: 139: RTT=8.9 [ms: 192.7 (91%, M+2H⁺), 213.2 (M+ACN+2H⁺), 384.3 (88%, M+H⁺)]

### Example 33

### Preparation of Products 141 to 144, 268 and 269

Product 109 to 112, 242 or 243, respectively, is dissolved in dry DMF, 40 eq. NaH are added and stirred at RT for 30 min. Cyclopentyl bromide is added to give a ratio of DMF:cyclopentyl bromide=1:1 and the reaction mixture is stirred over night. The reaction mixture is quenched with water and extracted with CH₂Cl₂. The combined organic phases are dried over MgSO₄, filtered and evaporated in vac. to dryness yielding 141 to 144, 268 or 269, respectively. Product 143 is purified by CC method D.

HPLC/MS Method B: 143: RTT=13.1 [ms: 372.3 (M+H⁺)]; 269: RTT=13.5 [ms: 386.4 (M+H⁺)]

### Example 34

### Preparation of Products 145 to 148, 270 and 271

Product 109 to 112, 242 or 243, respectively, is dissolved in dry DMF, 40 eq. NaH are added and stirred at RT for 30 min. 2-bromopropane is added to give a ratio of DMF:2-bromo-propane=1:1 and the reaction mixture is stirred over night. The reaction mixture is quenched with water and extracted with CH₂Cl₂. The combined organic phases are dried over MgSO₄, filtered and evaporated in vac. to dryness yielding 145 to 148, 270 or 271 respectively. Product 147 and 271 is purified by CC method D.

HPLC/MS Method B: 147: RTT=11.8 [ms: 346.3 (M+H⁺)]; 271: RTT=12.1 [ms: 360.3 (M+H⁺)]

### Example 35

### Preparation of Products 149 and 150 Following the Procedure of Frank D. King, J. Chem. Soc. Perkin Trans. 1, 447-453 (1986).

Anhydrous hydrogen chloride gas is bubbled through 3-butene-2-one containing a catalytical amount of dicinnamalacetone under cooling. After the reaction mixture turned red the formed 4-chloro-2-butanone is purified by distillation (bp: 62° C. at 92 mbar). 4-chloro-2-butanone is dissolved in CH₂Cl₂, 1 eq. 2,2-dimethyl-1,3-propanediol, 1 eq. triethylorthoformate and catalytic amounts of p-toluensulfonic acid are added and the reaction mixture is stirred over night. The reaction mixture is concentrated in vac., poured into saturated sodium bicarbonate solution and extracted with CH₂Cl₂. The combined organic phases are dried over anhydrous potassium carbonate, filtered and concentrated in vac. The formed 2-(2-chloroethyl)-2,5,5-trimethyl-1,3-dioxane is purified by distillation (bp: 86-90° C. at 22 mbar).

Methylphenylacetonitrile is added to a suspension of dry DMF and 2.3 eq. NaH at 70° C. in a dry and inert atmosphere (Argon). After stirring at 70° C. for 1 h 1.2 eq. 2-(2-chloroethyl)-2,5,5-trimethyl-1,3-dioxane are added drop wise. After stirring at 70° C. for 3 h and cooling to RT, the reaction mixture is poured into 1 L of ice-water. The product is extracted with Et₂O, the organic phase is dried over Na₂SO₄ filtered and evaporated in vac. to dryness yielding crude product which is directly used in the next step. To a cooled suspension (5° C.) of dry DEE and 1.5 eq. LAH in a dry and inert atmosphere crude product from the previous step dissolved in dry DEE is added drop wise. After stirring the reaction mixture at RT over night it is cooled with ice-water and water is added drop wise until no further gas formation is observed. The formed precipitate is filtered of, the filtrate is dried over Na₂SO₄ filtered and evaporated in vac. to dryness yielding a crude amine which is dissolved in Et₂O. 1 eq. 3-butene-2-one is added and the reaction mixture is stirred at RT for 2 h. The reaction mixture is added drop wise to 2.5 M HCl solution. The phases are separated and the aqueous phase is taken and refluxed for 4 h. After the addition of ice cubes, the mixture is neutralized with solid sodium carbonate and extracted with CH₂Cl₂. The combined organic layers are dried over Na₂SO₄ and the solvent is removed in vac. to dryness yielding 149 and 150. Product 149 and 150 are purified by CC method D.

HPLC/MS Method B: 149: RTT=8.6 [ms: 258.3 (M+H⁺), 276.2 (3%, M+H₃O⁺)]; 150: RTT=9.2 [ms: 258.3 (M+H⁺), 276.3 (3%, M+H₃O⁺)]

### Example 36

### Preparation of Products 151 and 152 Following the Procedure of Frank D. King, J. Chem. Soc. Perkin Trans. 1, 447-453 (1986).

Anhydrous hydrogen chloride gas is bubbled through 3-butene-2-one containing a catalytical amount of dicinnamalacetone under cooling. After the reaction mixture turned red the formed 4-chloro-2-butanone is purified by distillation (bp: 62° C. at 92 mbar). 4-chloro-2-butanone is dissolved in CH₂Cl₂, 1 eq. 2,2-dimethyl-1,3-propanediol, 1 eq. triethylorthoformate and catalytic amounts of p-toluensulfonic acid are added and the reaction mixture is stirred over night. The reaction mixture is concentrated in vac., poured into saturated sodium bicarbonate solution and extracted with CH₂Cl₂. The combined organic phases are dried over anhydrous potassium carbonate, filtered and concentrated in vac. The formed 2-(2-chloroethyl)-2,5,5-trimethyl-1,3-dioxane is purified by distillation (bp: 86-90° C. at 22 mbar).

4-Chloroacetophenone is dissolved in dry DME, abs. ethanol (catalytic amount) and 1.3 eq. p-toluenesulfonylmethylisocyanide are added. 2 eq. potassium-tert-butoxide are added in 4 portions under stirring and cooling. After stirring at RT over night the reaction mixture is filtered and the filtrate is concentrated in vac. The concentrated reaction mixture is filtrated over aluminum oxide and extracted with PE. The filtrate is evaporated in vac. to dryness yielding the desired 2-(4-chlorophenyl)propanenitrile.

2-(4-chlorophenyl)propanenitrile is added to a suspension of dry DMF and 2.3 eq. NaH at 70° C. in a dry and inert atmosphere (Argon). After stirring at 70° C. for 1 h 1.2 eq. 2-(2-chloroethyl)-2,5,5-trimethyl-1,3-dioxane are added drop wise. After stirring at 70° C. for 3 h and cooling to RT, the reaction mixture is poured into 1 L of ice-water. The product is extracted with Et₂O, the organic phase is dried over Na₂SO₄ filtered and evaporated in vac. to dryness yielding crude product which is directly used in the next step. To a cooled suspension (5° C.) of dry DEE and 1.5 eq. LAH in a dry and inert atmosphere crude product from the previous step dissolved in dry DEE is added drop wise. After stirring the reaction mixture at RT over night it is cooled with ice-water and water is added drop wise until no further gas formation was observed. The formed precipitate is filtered of, the filtrate is dried over Na₂SO₄ filtered and evaporated in vac. to dryness yielding a crude amine which is purified by CC method E. Purified amine from the previous step is dissolved in Et₂O, 1 eq. 3-butene-2-one is added and the reaction mixture is stirred at RT for 2 h. The reaction mixture is added drop wise to 2.5 M HCl solution. The phases are separated and the aqueous phase is taken and refluxed for 4 h. After addition of ice cubes, the mixture is neutralized with solid sodium carbonate and extracted with CH₂Cl₂. The combined organic layers are dried over Na₂SO₄ and the solvent is removed in vac. to dryness yielding 151 and 152. Product 151 and 152 are purified by CC method F.

HPLC/MS Method B: 151: RTT=10.7 [ms: 292.2, 294.2 (42%) (M+H⁺); 310.2 (9%, M+H₃O⁺)]; 152: RTT=11.1 [ms: 292.2, 294.2 (37%) (M+H⁺); 310.2 (4%, M+H₃O⁺)]

### Example 37

### Preparation of Products 153 and 154 Following the Procedure of Frank D. King, J. Chem. Soc. Perkin Trans. 1, 447-453 (1986)

Anhydrous hydrogen chloride gas is bubbled through 3-butene-2-one containing a catalytical amount of dicinnamalacetone under cooling. After the reaction mixture turned red the formed 4-chloro-2-butanone is purified by distillation (bp: 62° C. at 92 mbar). 4-chloro-2-butanone is dissolved in CH₂Cl₂, 1 eq. 2,2-dimethyl-1,3-propanediol, 1 eq. triethylorthoformate and catalytic amounts of p-toluensulfonic acid are added and the reaction mixture is stirred over night. The reaction mixture is concentrated in vac., poured into saturated sodium bicarbonate solution and extracted with CH₂Cl₂. The combined organic phases are dried over anhydrous potassium carbonate, filtered and concentrated in vac. The formed 2-(2-chloroethyl)-2,5,5-trimethyl-1,3-dioxane is purified by distillation (bp: 86-90° C. at 22 mbar).

p-Fluorobenzyl cyanide is added to a suspension of dry DMF and 1.03 eq. NaH at 70° C. in a dry and inert atmosphere (Argon). After stirring at 80° C. for 1 h 1.1 eq. 2-(2-chloroethyl)-2,5,5-trimethyl-1,3-dioxane are added drop wise. After stirring at 80° C. for 1.5 h the reaction mixture is cooled to 70° C., 1.03 eq. sodium hydride are added and the mixture is stirred for 1 h at 70° C. The reaction mixture is cooled to RT and 5 eq. MeI are added drop wise. After stirring the reaction mixture for 2 h at 35° C., the reaction mixture is poured into 1 L of ice-water. The product is extracted with Et₂O, the organic phase is dried over Na₂SO₄ filtered and evaporated in vac. to dryness yielding crude product which is directly used in the next step. To a cooled suspension (5° C.) of dry DEE and 1.5 eq. LAH in a dry and inert atmosphere crude product from the previous step dissolved in dry DEE is added drop wise. After stirring the reaction mixture at RT over night it is cooled with ice-water and water is added drop wise until no further gas formation is observed. The formed precipitate is filtered of, the filtrate is dried over Na₂SO₄ filtered and evaporated in vac. to dryness yielding a crude amine which is purified by CC method E. Purified amine from the previous step is dissolved in Et₂O, 0.5 eq. 3-butene-2-one is added and the reaction mixture is stirred at RT for 2 h. The reaction mixture is added drop wise to 2.5 M HCl solution. The phases are separated and the aqueous phase is taken and refluxed for 2.5 h. After addition of ice cubes, the mixture is neutralized with solid sodium carbonate and extracted with CH₂Cl₂. The combined organic layers are dried over Na₂SO₄ and the solvent is removed in vac. to dryness yielding 153 and 154. Product 153 and 154 are purified by CC method F.

HPLC/MS Method B: 153: RTT=9.2 [ms: 276.2 (M+H⁺)]; 154: RTT=9.5 [ms: 276.2 (M+H⁺)]

### Example 38

### Preparation of Products 163 and 164

Product 157 or 158 respectively, is dissolved in methanol, 2 eq. N-methylpiperazine and 2 eq. acetic acid are added. After 1 h 4 eq. sodium cyanoborohydride are added and the reaction mixture is stirred at RT over night. The reaction mixture is poured into a saturated sodium bicarbonate solution and extracted with CH₂Cl₂. The organic phase is dried over MgSO₄, filtered and evaporated in vac. to dryness yielding 163 or 164, respectively. Product 164 is purified by CC method E.

HPLC/MS Method B: 164: RTT=2.6 [ms: 200.8 (63% M+2H⁺), 221.3 (37%, M+ACN+2H⁺), 400.3 (M+H⁺)]

### Example 39

### Preparation of Products 165, 166, 193 and 194

Product 157, 158, 191 or 192 respectively, is dissolved in DMF/AcOH=9:1, 10 eq. 3-amino-1,2,4-triazole are added and the reaction mixture is heated to 95° C. for 2 h. After cooling to RT 10 eq. sodium cyanoborohydride are added and the reaction mixture is stirred at 50° C. over night. The reaction mixture is poured into a saturated sodium bicarbonate solution and extracted with CH₂Cl₂. The organic phase is dried over MgSO₄, filtered and evaporated in vac. to dryness yielding 165, 166, 193 or 194, respectively. Product 166 and 194 is purified by CC method E.

HPLC/MS Method B: 166: RTT=7.3 [ms: 192.8 (10%, M+2H⁺), 213.3 (36%, M+ACN+2H⁺), 366.2 (9%, M+H⁺—H₂O), 384.2 (M+H⁺)]; 194: RTT=7.9 [ms: 201.7 (63%, M+2H⁺), 222.3 (83%, M+ACN+2H⁺), 384.3 (3%, M+H⁺—H₂O), 402.3 (M+H⁺)]

### Example 40

### Preparation of Products 167 and 168

Product 157 or 158, respectively, is dissolved in DMF/AcOH=9:1, 10 eq. 5-(trifluoromethyl)-1,2,4-triazol-3-amine are added and the reaction mixture is heated to 95° C. for 2 h. After cooling to RT 10 eq. sodium cyanoborohydride are added and the reaction mixture is stirred at 50° C. over night. The reaction mixture is poured into a saturated sodium bicarbonate solution and extracted with CH₂Cl₂. The organic phase is dried over MgSO₄, filtered and evaporated in vac. to dryness yielding 167 or 168 respectively. Product 168 is purified by CC method E.

HPLC/MS Method B: 168: RTT=11.7 [ms: 452.3 (M+H⁺)]

### Example 41

### Preparation of Products 169 and 170

Morpholine is dissolved in CH₂Cl₂ and acetic anhydride is added drop wise under ice-water cooling. After stirring at RT over night the reaction mixture is washed once with distilled water, once with 15% HCl solution and finally with saturated sodium carbonate solution. The organic phase is dried over MgSO₄, filtered and evaporated in vac. to dryness yielding 4-acetylmorpholine sufficiently pure for further conversion.

1.6 eq. 4-acetylmorpholine are dissolved in a dry inert atmosphere (Argon) in dry THF and cooled to −78° C. 1.9 eq. LDA solution are added and kept for 20 min at −78° C. Product 149 or 150, respectively, is dissolved in dry THF and added at −78° C. to the reaction mixture. After stirring the reaction mixture for 1 h at RT the reaction mixture is hydrolyzed with aq. NH₄Cl solution and extracted with Et₂O. The organic phase is dried over MgSO₄, filtered and evaporated in vac. to dryness yielding 169 or 170, respectively. Product 170 is purified by CC method D.

HPLC/MS Method B: 170: RTT=10.5 [ms: 387.2 (M+H⁺)]

### Example 42

### Preparation of Products 171 and 172

Product 169 or 170, respectively, is dissolved in a dry inert atmosphere (Argon) in dry THF and cooled to −78° C. 2 eq. LAH are added at −78° C. and the reaction mixture is stirred at RT over night. 5 eq. of potassium sodium tartrate tetrahydrate are added, the precipitate is filtered of and the filtrate is evaporated in vac. to dryness yielding 171 or 172, respectively. Product 172 is purified by CC method E.

HPLC/MS Method B: 172: RTT=4.7 [ms: 187.2 (96%, M+2H⁺), 207.9 (18%, M+CAN+2H⁺), 373.2 (M+H⁺)]

### Example 43

### Preparation of Products 173 and 174

Pyrrolidine is dissolved in CH₂Cl₂ and 1 eq. TEA is added. 1.2 eq. acetyl chloride are added drop wise under ice-water cooling. After stirring at RT over night the reaction mixture is poured into saturated Na₂CO₃ solution and extracted with CH₂Cl₂. The organic phase is dried over MgSO₄, filtered and evaporated in vac. to dryness yielding N-acetylpyrrolidine sufficiently pure for further conversion.

10 eq. N-acetylpyrrolidine are dissolved in a dry inert atmosphere (Argon) in dry THF and cooled to −70° C. 10 eq. LDA solution are added and kept for 1 h at −70° C. Product 149 or 150, respectively, is dissolved in dry THF and added at −70° C. to the reaction mixture which is kept at −70° C. over night. After warming to RT the reaction mixture is hydrolyzed with aq. NH₄Cl solution and extracted with CH₂Cl₂. The organic phase is dried over MgSO₄, filtered and evaporated in vac. to dryness yielding 173 or 174, respectively. Product 174 is purified by CC method D.

HPLC/MS Method B: 174: RTT=11.1 [ms: 371.3 (M+H⁺)]

### Example 44

### Preparation of Products 175 and 176

Product 173 or 174, respectively, is dissolved in a dry inert atmosphere (Argon) in dry THF and cooled to 0° C. 2 eq. LAH are added at 0° C. and the reaction mixture is stirred at RT over night. 5 eq. of potassium sodium tartrate tetrahydrate are added, the precipitate is filtered of and the filtrate is evaporated in vac. to dryness yielding 175 or 176, respectively.

HPLC/MS Method B: 176: RTT=5.1 [ms: 179.3 (M+2H⁺), 199.7 (16%, M+CAN+2H⁺), 357.3 (42%, M+H⁺)]

### Example 45

### Preparation of Products 179 to 182

Product 149 or 150, respectively, is dissolved in dry DME, abs. ethanol (catalytic amount) and 1.3 eq. p-toluenesulfonylmethylisocyanide are added. 4 eq. potassium-tert-butoxide are added in portions under stirring and cooling. After stirring at RT over night the reaction mixture is diluted with water and extracted Et₂O. The organic phase is dried over MgSO₄, filtered and evaporated in vac. to dryness yielding 179 and 180 or 181 and 182, respectively. Product 181 and 182 are purified by CC method F.

HPLC/MS Method B: 181: RTT=9.9 [ms: 269.2 (M+H⁺)]; 182: RTT=10.3 [ms: 269.3 (M+H⁺)]

### Example 46

### Preparation of Products 195 and 196

Product 157 or 158, respectively, is dissolved in DMF/AcOH=9:1, 10 eq. 5-methyl-1,2,4-triazol-3-amine are added and the reaction mixture is heated to 50° C. for 5 h. After cooling to RT 10 eq. sodium cyanoborohydride are added and the reaction mixture is stirred at RT over night. The reaction mixture is poured into a saturated Na₂CO₃ solution and extracted with CH₂Cl₂. The organic phase is dried over MgSO₄, filtered and evaporated in vac. to dryness yielding 195 or 196 respectively. Product 196 is purified by CC method E.

HPLC/MS Method B: 196: RTT=7.5 [ms: 199.8 (M+2H⁺), 220.4 (42%, M+ACN+2H⁺), 380.3 (3%, M+H⁺—H₂O), 398.3 (59%, M+H⁺)]

### Example 47

### Preparation of 197 Following the Procedure of Frank D. King, J. Chem. Soc. Perkin Trans. 1, 447-453 (1986)

To a suspension of dry DMF and 2.3 eq. NAH at 70° C. in a dry and inert atmosphere (Argon) diphenylacetonitrile is added. After stirring at 70° C. for 1 h 1.05 eq. 3-chloropropion-aldehyde diethyl acetal are added drop wise. After stirring at 70° C. for 1 h and cooling to RT, the reaction mixture is poured into of ice-water. The product is extracted with Et₂O, the organic phase is dried over Na₂SO₄, filtered and evaporated in vac. to dryness. The crude product is directly used in the next step. To a suspension of 0.9 eq. LAH in dry THF 0.4 eq concentrated H₂SO₄ is added drop wise under cooling in a dry and inert atmosphere (Argon). After stirring at 0° C. for 1 h, a solution of the crude product from the previous step in dry THF is added drop wise and the reaction mixture is stirred at RT for 5 h. After cooling to 0° C. 1 M NaOH solution is added, the formed precipitate is removed by suction filtration and Et₂O is used to wash the precipitate. The filtrate is evaporated in vac. to dryness yielding crude amine, which is dissolved in Et₂O. 1 eq. 3-buten-2-one is added and the reaction mixture is stirred at RT for 2 h. The reaction mixture is added drop wise to a 2.5 M HCl solution and the aqueous phase is refluxed for 2 h. After addition of ice cubes, the mixture is neutralized with Na₂CO₃ and extracted with CH₂Cl₂. The combined organic layers are dried over Na₂SO₄ and the solvent is removed in vac. to dryness yielding 197 which is purified by CC method B. MS direct infusion: 197: [ms: 324.2 (M+H₃O⁺), 306.2 (60%, M+H⁺)]

### Example 48

### Preparation of Product 199

Product 197 is dissolved in methanol, 2 eq. piperidine and 2 eq. acetic acid are added. After 1 h 4 eq. sodium cyanoborohydride are added and the reaction mixture is stirred at RT over night. The reaction mixture is poured into a saturated NaHCO₃ solution and extracted with CH₂Cl₂. The organic phase is dried over MgSO₄, filtered and evaporated in vac. to dryness yielding 199 which is purified by CC method D.

MS direct infusion: 199: [ms: 375.3 (M+H⁺)]

### Example 49

### Preparation of Products 200 and 201

Product 3 or 4, respectively, is dissolved in dry DMF, 5 eq. NaH are added and stirred at RT for 45 min. 5 eq. isobutyl bromide are added and the reaction mixture is stirred over night. The reaction mixture is quenched with water and extracted with CH₂Cl₂. The combined organic phases are dried over MgSO₄, filtered and evaporated in vac. to dryness yielding 200 or 201 respectively. Product 201 is purified by CC method D.

HPLC/MS Method A: 201: Stereoisomer I: RTT=10.8 [ms: 302.1 (M+H⁺)], Stereoisomer II: RTT=11.9 [ms: 302.2 (M+H⁺)]

### Example 50

### Preparation of Products 204 to 207

Product 1 or 2, respectively, is dissolved in dry ethane-1,2-diol, conc. H₂SO₄ (2%) is added and the reaction mixture is stirred for 3 days at RT. The reaction mixture is diluted with water, neutralized with Na₂CO₃ and extracted with CH₂Cl₂. The organic phase is dried over MgSO₄, filtered and evaporated in vac. to dryness yielding 204 and 206 or 205 and 207, respectively. Product 205 and 207 are purified by CC method C.

HPLC/MS Method A: 205: RTT=8.1 [ms: 288.1 (M+H⁺)]; 207: RTT=5.1 [ms: 350.1 (M+H⁺), 288.1 (78%, M−HOCH₂CH₂OH+H⁺), 372.1 (8%, (M+Na⁺)]

### Example 51

### Preparation of Products 210 to 213

Product 179 to 182, respectively, is dissolved in dry THF in a dry and inert atmosphere (Argon) and cooled to −78° C. 5 eq. DIBAL-H solution are added and the mixture is kept at −78° C. over night. The reaction mixture is poured into 1.2 M HCl solution and stirred for 15 min at RT. The reaction mixture is rendered alkaline with Na₂CO₃ and extracted with Et₂O. The organic phase is dried over MgSO₄, filtered and evaporated in vac. to dryness yielding 210 to 213, respectively. Product 212 is purified by CC method D.

HPLC/MS Method B: 212: RTT=9.3 [ms: 290.3 (M+H₃O⁺), 272.3 (37%, M+H⁺)]

### Example 52

### Preparation of Products 214 to 217

Product 179 to 182, respectively, is dissolved in dry THF and added drop wise to a cooled suspension (5° C.) of dry THF and 1.5 eq. LAH in a dry and inert atmosphere (Argon). After stirring the reaction mixture at RT over night it is cooled with ice-water and water is added drop wise until no further gas formation is observed. The formed precipitate is filtered of, the filtrate is dried over Na₂SO₄, filtered and evaporated in vac. to dryness yielding 214 to 217, respectively. Product 216 is purified by CC method D.

HPLC/MS Method B: 216: RTT=3.7 [ms: 157.8 (15% M+ACN+2H⁺), 273.3 (M+H⁺)]

### Example 53

### Preparation of Products 218 to 221, 236 and 237

Product 210 to 213, 234 or 235, respectively, is dissolved in methanol, 7 eq. 3-amino-1,2,4-triazole and 7 eq. acetic acid are added. After stirring the reaction mixture at RT over night 8 eq. sodium cyanoborohydride are added. After stirring for 4 h at RT the reaction mixture is poured into saturated NaHCO₃ solution and extracted with CH₂Cl₂. The combined organic phases are dried over MgSO₄, filtered and evaporated in vac. to dryness yielding 218 to 221, 236 or 237, respectively. Product 220 and 237 is purified by CC method E.

HPLC/MS Method B: 220: RTT=8.0 [ms: 170.8 (23%, M+2H⁺), 191.3 (M+ACN+2H⁺), 340.3 (77%, M+H⁺)]; 237: RTT=9.2 [ms: 199.8 (63%, M+2H⁺), 220.4 (M+ACN+2H⁺), 398.4 (62%, M+H⁺)]

### Example 54

### Preparation of Products 222 to 225, 276 and 277

Product 125 to 128, 234 or 235, respectively, is dissolved in methanol, 10 eq. 2-amino-thiazole, 5 eq. acetic acid and MgSO₄ are added. After stirring the reaction mixture at 50° C. over night 1.5 eq. sodium cyanoborohydride are added at RT. After stirring for 7 h at RT the reaction mixture is diluted with water, rendered alkaline with Na₂CO₃ and extracted with CH₂Cl₂. The combined organic phases are dried over MgSO₄, filtered and evaporated in vac. to dryness yielding 222 to 225, 276 or 277, respectively. Product 224 is purified by CC method E.

HPLC/MS Method B: 224: RTT=9.5 [ms: 200.9 (M+2H⁺), 221.4 (70%, M+ACN+2H⁺), 400.3 (40%, M+H⁺)]; 277: RTT=9.7 [ms: 207.9 (M+2H⁺), 228.4 (55%, M+ACN+2H⁺), 414.3 (31%, M+H⁺)]

### Example 55

### Preparation of Products 226 and 227

Product 157 or 158, respectively, is dissolved in methanol, 2 eq. cyclopentylamine and 2 eq. acetic acid are added. After stirring the reaction mixture for 2 h at RT 4 eq. sodium cyanoborohydride are added and the reaction mixture is stirred at RT over night. The reaction mixture is diluted with water, rendered alkaline with Na₂CO₃ and extracted with CH₂Cl₂. The combined organic phases are dried over MgSO₄, filtered and evaporated in vac. to dryness yielding 226 or 227, respectively. Product 227 is purified by CC method E.

HPLC/MS Method B: 227: RTT=8.1 [ms: 193.4 (M+2H⁺), 213.9 (99%, M+ACN+2H⁺), 385.4 (47%, M+H⁺)]

### Example 56

### Preparation of Products 228 to 231, 274 and 275

Product 125 to 128, 234 or 235, respectively, is dissolved in methanol, 5 eq. 2-amino-pyrimidine, 5 eq. acetic acid and MgSO₄ are added. After stirring the reaction mixture at 50° C. over night 1.5 eq. sodium cyanoborohydride are added at RT. After stirring for 7 h at RT the reaction mixture is diluted with water, rendered alkaline with Na₂CO₃ and extracted with CH₂Cl₂. The combined organic phases are dried over MgSO₄, filtered and evaporated in vac. to dryness yielding 228 to 231, 274 or 275, respectively. Product 230 is purified by CC method E.

HPLC/MS Method B: 230: RTT=11.0 [ms: 198.3 (51%, M+2H⁺), 218.9 (M+ACN+2H⁺), 395.3 (51%, M+H⁺)]; 275: RTT=11.3 [ms: 205.4 (46%, M+2H⁺), 225.9 (M+ACN+2H⁺), 409.4 (59%, M+H⁺)]

### Example 57

### Preparation of Products 232 and 233

Product 155 or 156, respectively, is dissolved in dry DMF, 4 eq. NaH are added and the reaction mixture is stirred at RT for 2 h. 10 eq. MeI are added and the reaction mixture is stirred for 4 h.

The reaction mixture is quenched with water and extracted with Et₂O. The combined organic phases are dried over MgSO₄, filtered and evaporated in vac. to dryness yielding 232 or 233, respectively. Product 233 is purified by CC method D.

HPLC/MS Method B: 233: RTT=12.6 [ms: 388.4 (M+H⁺)]

### Example 58

### Preparation of Products 234 and 235

Product 232 or 233, respectively, is dissolved in a 5% aqueous HCl solution and stirred for 4 h at 90° C. After cooling to RT the reaction mixture is diluted with water, rendered alkaline with Na₂CO₃ (pH 11) and extracted with CH₂Cl₂. The organic phase is dried over MgSO₄, filtered and evaporated in vac. to dryness yielding 234 or 235, respectively.

HPLC/MS Method B: 235: RTT=11.2 [ms: 330.3 (M+H⁺), 348.3 (40%, M+H₃O⁺)]

### Example 59

### Preparation of Products 238 and 239

To a stirred solution of 1.5 eq. dry ethyl acetate in dry THF 1.5 eq. LDA solution is added drop wise at −77° C. in a dry and inert atmosphere (Argon). After stirring for 1 h at −77° C. this reaction mixture is added to a cooled solution of product 149 or 150, respectively, in dry THF at −77° C. The reaction mixture is stirred for 1 h at −77° C., quenched with a saturated NH₄Cl solution and then warmed to RT. The mixture is extracted with Et₂O, the organic phase is dried over MgSO₄, filtered and evaporated in vac. to dryness yielding 238 or 239, respectively. Product 239 is purified by CC method G.

HPLC/MS Method B: 239: RTT=11.4 [ms: 346.3 (M+H⁺)]

### Example 60

### Preparation of Products 240 and 241

To a stirred solution of 5 eq. dry acetonitrile in dry THF 5 eq. LDA solution is added drop wise at −77° C. in a dry and inert atmosphere (Argon). After stirring for 1 h at −77° C. this reaction mixture is added to a cooled solution of product 149 or 150, respectively, in dry THF at −77° C. The reaction mixture is stirred for 1 h at −77° C., quenched with a saturated NH₄Cl solution and then warmed to RT. The reaction mixture is extracted with Et₂O, the organic phase is dried over MgSO₄, filtered and evaporated in vac. to dryness yielding 240 or 241, respectively. Product 241 is purified by CC method G.

HPLC/MS Method B: 241: RTT=9.8 [ms: 299.3 (M+H⁺)]

### Example 61

### Preparation of Products 244 to 247

Product 210 to 213, respectively, is dissolved in methanol, 7 eq. 5-(trifluoromethyl)-1,2,4-triazol-3-amine and 7 eq. acetic acid are added. After stirring the reaction mixture at RT over night 8 eq. sodium cyanoborohydride are added. After stirring for 4 h at RT the reaction mixture is poured into saturated NaHCO₃ solution and extracted with CH₂Cl₂. The combined organic phases are dried over MgSO₄, filtered and evaporated in vac. to dryness yielding 244 to 247, respectively. Product 246 is purified by CC method E.

HPLC/MS Method B: 246: RTT=12.3 [ms: 408.3 (M+H⁺)]

### Example 62

### Preparation of Products 250 to 253

Product 125 to 128, respectively, is dissolved in methanol, 10 eq. 2-aminooxazole, 5 eq. acetic acid and MgSO₄ are added. After stirring the reaction mixture at 50° C. over night 1.5 eq. sodium cyanoborohydride are added at RT. After stirring for 7 h at RT the reaction mixture is diluted with water, rendered alkaline with Na₂CO₃ and extracted with CH₂Cl₂. The combined organic phases are dried over MgSO₄, filtered and evaporated in vac. to dryness yielding 250 to 253, respectively.

HPLC/MS Method B: 252: RTT=9.4 [ms: 192.9 (M+2H⁺), 213.4 (89%, M+ACN+2H⁺), 384.3 (75%, M+H⁺)]

### Example 63

### Preparation of Products 254 to 257

Product 125 to 128, respectively, is dissolved in methanol, 10 eq. 2-aminobenzimidazole, 5 eq. acetic acid and MgSO₄ are added. After stirring the reaction mixture at 50° C. over night 1.5 eq. sodium cyanoborohydride are added at RT. After stirring for 7 h at RT the reaction mixture is diluted with water, rendered alkaline with Na₂CO₃ and extracted with CH₂Cl₂. The combined organic phases are dried over MgSO₄, filtered and evaporated in vac. to dryness yielding 254 to 257, respectively.

HPLC/MS Method B: 256: RTT=10.6 [ms: 217.2 (M+2H⁺), 237.9 (35%, M+ACN+2H⁺), 433.4 (55%, M+H⁺)]

### Example 64

### Preparation of Products 258 to 261

Product 125 to 128, respectively, is dissolved in 1,2 dichloroethane, 5 eq. 2,5-dimethylpyrrole, 5 eq. acetic acid and MgSO₄ are added. After stirring the reaction mixture at 70° C. over night 1.5 eq. sodium cyanoborohydride are added at RT. After stirring for 4 h at RT the reaction mixture is diluted with water, rendered alkaline with Na₂CO₃ and extracted with CH₂Cl₂. The combined organic phases are dried over MgSO₄, filtered and evaporated in vac. to dryness yielding 258 to 261, respectively.

HPLC/MS Method B: 260: RTT=15.4 [ms: 395.3 (M+H⁺)]

### Example 65

### Preparation of Products 262 to 265

Product 214 to 217, respectively, is dissolved in dry DMF and 2 eq. TEA are added. After stirring for 5 min at RT 1.3 eq. 1-pyrrolidinecarbonyl chloride are added and the reaction mixture is stirred over night at RT. The reaction mixture is quenched with water, rendered alkaline with Na₂CO₃ and extracted with CH₂Cl₂. The combined organic phases are dried over MgSO₄, filtered and evaporated in vac. to dryness yielding 262 to 265, respectively. Product 264 is purified by CC method E.

HPLC/MS Method B: 264: RTT=11.3 [ms: 370.4 (M+H⁺), 392.3 (5%, M+Na⁺)]

### Example 66

### Preparation of Products 266 and 267

Product 242 or 243, respectively, is dissolved in dry DMF, 10 eq. NaH are added and stirred at RT for 1 h. 5 eq. MeI are added and the reaction mixture is stirred for 3 h. The reaction mixture is quenched with water and extracted with Et₂O. The combined organic phases are dried over MgSO₄, filtered and evaporated in vac. to dryness yielding 266 or 267, respectively.

HPLC/MS Method B: 267: RTT=12.2 [ms: 346.3 (M+H⁺)]

### Example 67

### Preparation of Products 278 and 279

Product 242 or 243, respectively, is dissolved in dry DMF, 2.5 eq. NaH are added and stirred at RT for 1 h. 1 eq. MeI is added and the reaction mixture is stirred for 2 h. The reaction mixture is quenched with water and extracted with Et₂O. The combined organic phases are dried over MgSO₄, filtered and evaporated in vac. to dryness yielding 278 or 279, respectively. Product 279 is purified by CC method D.

HPLC/MS Method B: 279: RTT=10.6 [ms: 332.3 (M+H⁺)]

### Example 68

### Preparation of Products 280 and 281

To a cooled (0° C.) suspension of 3 eq. LAH in dry THF in a dry inert atmosphere (Argon) 1.5 eq. H₂SO₄ in THF are added and stirred for 1 h at 0° C. Product 238 or 239, respectively, dissolved in dry THF is added at 0° C. After stirring over night at RT, 3 eq. of potassium sodium tartrate tetrahydrate are added to the reaction mixture, the precipitate is filtered of and the filtrate is evaporated in vac. to dryness yielding 280 or 281, respectively.

HPLC/MS Method B: 281: RTT=9.1 [ms: 304.2 (M+H⁺)]

### Example 69

### Preparation of Products 282 and 283

Product 238 or 239, respectively, is dissolved in THF, potassium hydroxide dissolved in methanol/water is added and the reaction mixture is stirred at RT for 2 days. The organic solvent is removed in vac. The aqueous phase is neutralized with a 6 N HCl solution and the solvent is removed by freeze drying yielding 282 or 283 respectively, as hydrochlorides.

HPLC/MS Method B: 283: RTT=9.1 [ms: 318.2 (M+H⁺)]

### Example 70

### Preparation of Products 284 and 285

Product 282 or 283, respectively, is dissolved in a dry inert atmosphere (Argon) in dry DMF, 5 eq. TEA, 10 eq. 3-amino-1,2,4-triazole and 10 eq. TBTU are added. After stirring the reaction mixture over night at RT, it is quenched at 0° C. with a saturated NaHCO₃ solution and extracted with CH₂Cl₂. The organic phase is dried over MgSO₄, filtered and evaporated in vac. to dryness yielding 284 or 285, respectively.

HPLC/MS Method B: 285: RTT=10.1 [ms: 384.2 (M+H⁺)]

### Example 71

### Preparation of Products 286 and 287

Product 236 or 237, respectively, is dissolved in dry DCM and 16 eq. boron tribromide solution is added at −78° C. After stirring the reaction mixture at RT for 1.5 h it is added drop wise to a stirred 15% Na₂CO₃ solution and extracted with CH₂Cl₂. The organic phase is dried over MgSO₄, filtered and evaporated in vac. to dryness. The crude product is dissolved in methanol, 10% Pd/C is added and the reaction mixture is stirred over night under hydrogen atmosphere. After filtering of the catalyst, the organic phase is dried over MgSO₄, filtered and evaporated in vac. to dryness yielding 286 or 287 respectively. Product 287 is purified by CC method E.

HPLC/MS Method B: 287: RTT=9.3 [ms: 184.9 (74%, M+2H⁺), 205.4 (M+ACN+2H⁺), 368.3 (71%, M+H⁺)]

### Example 72

### Preparation of Products 288, 289, 292 and 293

Product 296, 297, 294 or 295, respectively, is dissolved in methanol, 10 eq. morpholine and 10 eq. acetic acid are added. After stirring the reaction mixture for 2 h at RT 4 eq. sodium cyanoborohydride are added and the reaction mixture is stirred at RT for one week. The reaction mixture is diluted with water, rendered alkaline with Na₂CO₃ and extracted with CH₂Cl₂. The combined organic phases are dried over MgSO₄, filtered and evaporated in vac. to dryness yielding 288, 289, 292 or 293, respectively. Product 289 is purified by CC method E.

HPLC/MS Method B: 289: RTT=Isomer I: 7.5 [ms: 186.3 (M+2H⁺), 206.8 (81%, M+ACN+2H⁺), 371.3 (87%, M+H⁺)]; Isomer II: 8.0 [ms: 186.3 (M+2H⁺), 206.9 (88%, M+ACN+2H⁺), 371.3 (78%, M+H⁺)]; 293: RTT=5.6 [ms: 194.3 (M+2H⁺), 214.8 (26%, M+ACN+2H⁺), 387.3 (92%, M+H⁺)]

### Example 73

### Preparation of Products 290 and 310 or 291 and 311

Product 294 or 295, respectively, is dissolved in methanol, 10 eq. 3-amino-1,2,4-triazole and 10 eq. acetic acid are added. After stirring the reaction mixture over night at 50° C. 4 eq. sodium cyanoborohydride are added and the reaction mixture is stirred at RT for two weeks. The reaction mixture is diluted with water, rendered alkaline with Na₂CO₃ and extracted with CH₂Cl₂. The combined organic phases are dried over MgSO₄, filtered and evaporated in vac. to dryness yielding 290 and 310 or 291 and 311, respectively.

HPLC/MS Method B: 291: RTT=6.4 [ms: 192.8 (25%, M+2H⁺), 213.2 (34%, M+ACN+2H⁺), 384.3 (M+H⁺)]; 311: RTT=9.7 [ms: 318.2 (M+H⁺)]

### Example 74

### Preparation of Products 294 and 296 or 295 and 297

Product 240 or 241 respectively, is dissolved in a dry inert atmosphere (Argon) in dry DEE and 7 eq. methyl magnesium bromide solution are added at RT. After stirring the reaction mixture overnight it is acidified by addition of a 1% aqueous formic acid solution and stirred for 20 min. The mixture is rendered alkaline by addition of NaHCO₃ and extracted with Et₂O. The combined organic phases are dried over MgSO₄, filtered and evaporated in vac. to dryness. The crude product is dissolved in methanol, 10% Pd/C is added and the reaction mixture is stirred over night under hydrogen atmosphere. After filtering of the catalyst, the filtrate is evaporated in vac. to dryness yielding 294 and 296 or 295 and 297, respectively. Product 295 is purified by CC method D.

HPLC/MS Method B: 295: RTT=10.2 [ms: 316.3 (M+H⁺)], 297: RTT=11.3 [ms: 300.3 (M+H⁺)]

### Example 75

### Preparation of Products 298 to 301

Product 238 to 241, respectively, is dissolved in a dry inert atmosphere (Argon) in dry DMF, 30 eq. magnesium flaks and 50 eq. trimethylsilyl chloride are added and the reaction mixture is stirred over night at RT. The reaction mixture is quenched by addition of water containing 1% TEA and extracted with Et₂O. After washing the organic phase with brine and water it is dried over MgSO₄, filtered and evaporated in vac. to dryness yielding 298 to 301, respectively. Product 299 and 301 is purified by CC method D.

HPLC/MS Method B: 299: RTT=15.9 [ms: 418.3 (M+H⁺)], 301: RTT=15.1 [ms: 371.3 (M+H⁺)]

### Example 76

### Preparation of Products 304 and 305

Product 238 or 239, respectively, is dissolved in a dry inert atmosphere (Argon) in dry DEE and 4 eq. methyl magnesium bromide solution are added at RT. After stirring the reaction mixture for 3 h it is quenched with NH₄Cl solution and extracted with CH₂Cl₂. The combined organic phases are dried over MgSO₄, filtered and evaporated in vac. to dryness yielding 304 or 305, respectively.

HPLC/MS Method B: 305: RTT=10.5 [ms: 332.3 (M+H⁺)]

### Example 77

### Preparation of Products 306 to 309

Product 179 to 182, respectively, is dissolved in a dry inert atmosphere (Argon) in dry DEE and 5 eq. methyl magnesium bromide solution are added at RT. After stirring the reaction mixture over night it is added drop wise to a 0.1 M HCl solution and stirred for 10 min. The mixture is rendered alkaline by addition of Na₂CO₃ and extracted with Et₂O. The combined organic phases are dried over MgSO₄, filtered and evaporated in vac. to dryness. The crude product is dissolved in a dry inert atmosphere (Argon) in dry DEE, 5 eq. methyl magnesium bromide solution are added and the reaction mixture is heated to reflux for 2.5 h. The reaction mixture is quenched with a saturated NH₄Cl solution and extracted with Et₂O. The combined organic phases are dried over MgSO₄, filtered and evaporated in vac. to dryness yielding 306 to 309, respectively. Product 308 is purified by CC method D.

HPLC/MS Method B: 308: RTT=10.6 [ms: 302.3 (M+H⁺)]

### Example 78

### Preparation of Products 314 to 317

Product 179 to 182, respectively, is dissolved in 37% HCl and refluxed over night. The reaction mixture is diluted with water and freeze dried yielding 314 to 317 as hydrochlorides.

HPLC/MS Method B: 316: RTT=10.2 [ms: 288.3 (M+H⁺)]

### Example 79

### Preparation of Products 318 to 321

Product 109 to 112 respectively, is dissolved in toluene, 4% conc. H₂SO₄ is added and the reaction mixture is stirred at 50° C. for 2 h. The reaction mixture is poured into water, rendered alkaline with Na₂CO₃ and extracted with CH₂Cl₂. The combined organic phases are dried over MgSO₄, filtered and evaporated in vac. to dryness yielding 318 and 319 or 320 and 321, respectively. Product 320 and 321 is purified by CC method D.

HPLC/MS Method B: 318: RTT=11.0 [ms: 286.2 (M+H⁺)]; 319: RTT=10.1 [ms: 286.3 (M+H⁺)]; 320: RTT=11.6 [ms: 286.2 (M+H⁺)]; 321: RTT=11.0 [ms: 286.2 (M+H⁺)]

### Example 80

### Preparation of Products 322 and 323

Product 248 or 249, respectively, is dissolved in methanol containing 10% KOH. The reaction mixture is stirred for 2 h yielding 322 or 323, respectively.

HPLC/MS Method B: 323: RTT=9.8 [ms: 332.3 (M+H⁺)]

### Example 81

### Preparation of Products 324 and 325

Product 242 or 243, respectively, is dissolved in a dry inert atmosphere (Argon) in dry THF, 1.5 eq. triphenylphosphine and 1.5 eq. DEAD are added and the reaction mixture is stirred at RT for 1 h yielding 324 or 325, respectively.

HPLC/MS Method B: 325: RTT=12.3 [ms: 300.3 (M+H⁺)]

### Example 82

### Preparation of Products 326 and 327

Product 165 or 166, respectively, is dissolved in a dry inert atmosphere (Argon) in dry DMF and cooled to 0° C. A gentle stream of formaldehyde gas, which is produced by pyrolysis of paraformaldehyde (240 eq.), is bubbled through the mixture via a Teflon tube under stirring. After stirring the reaction mixture over night 38 eq. sodium cyanoborohydride are added and the reaction mixture is stirred for 2 h. The reaction mixture is poured into a NaHCO₃ solution and extracted with CH₂Cl₂. The organic phase is dried over MgSO₄, filtered and evaporated in vac. to dryness yielding 326 or 327, respectively.

HPLC/MS Method B: 327: RTT=9.0 [ms: 199.8 (M+2H⁺), 220.4 (75%, M+ACN+2H⁺), 398.3 (72%, M+H⁺)]

### Biological Methods

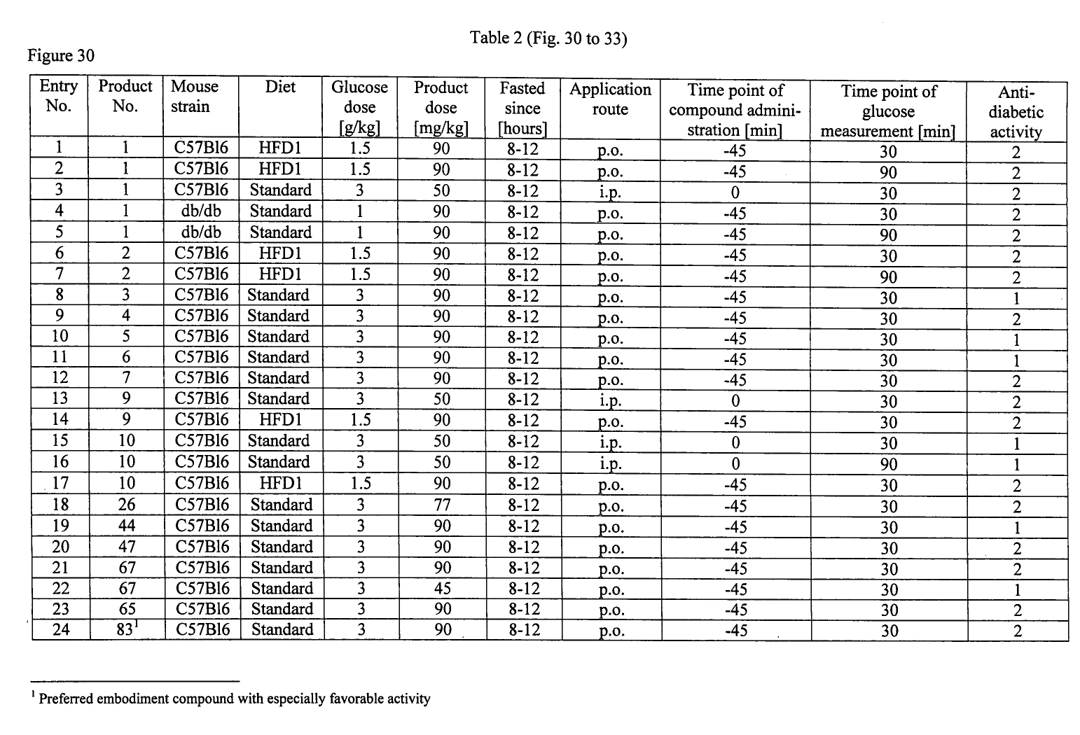

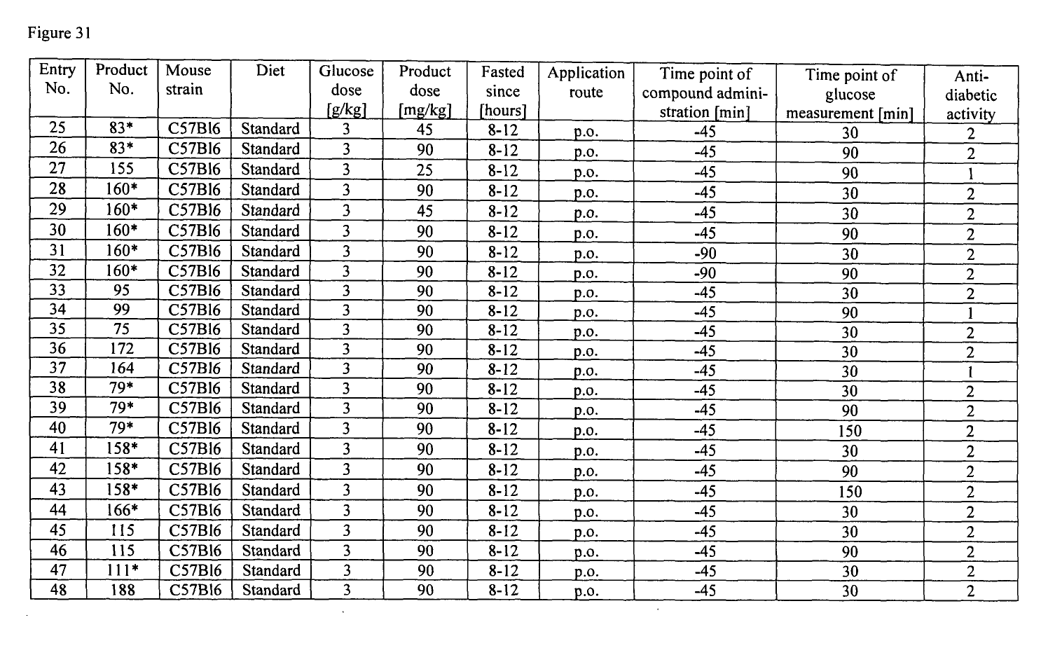

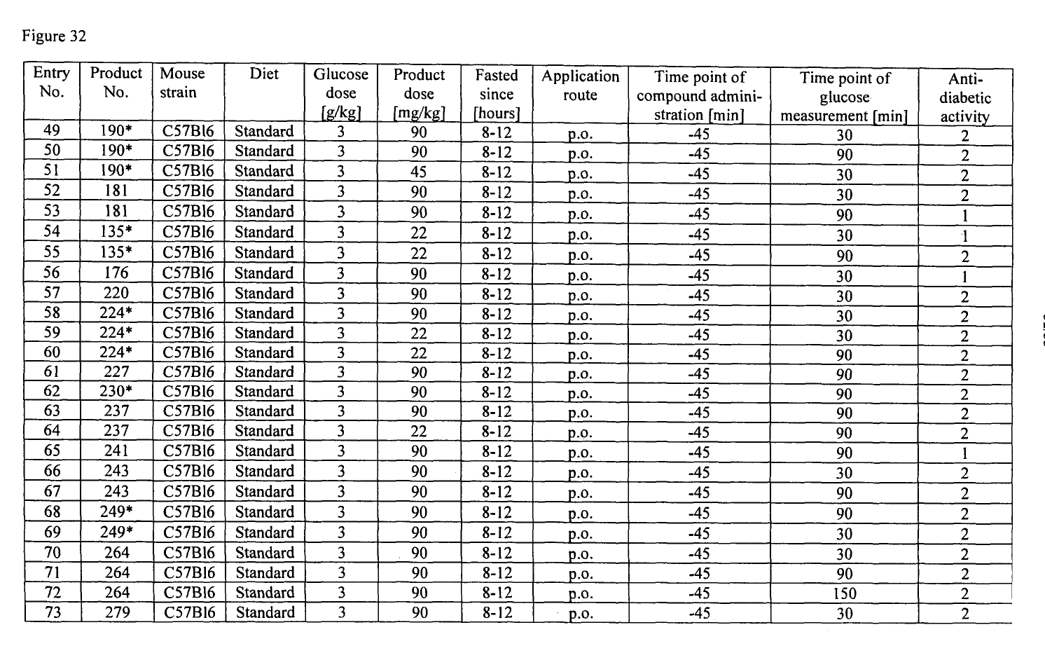

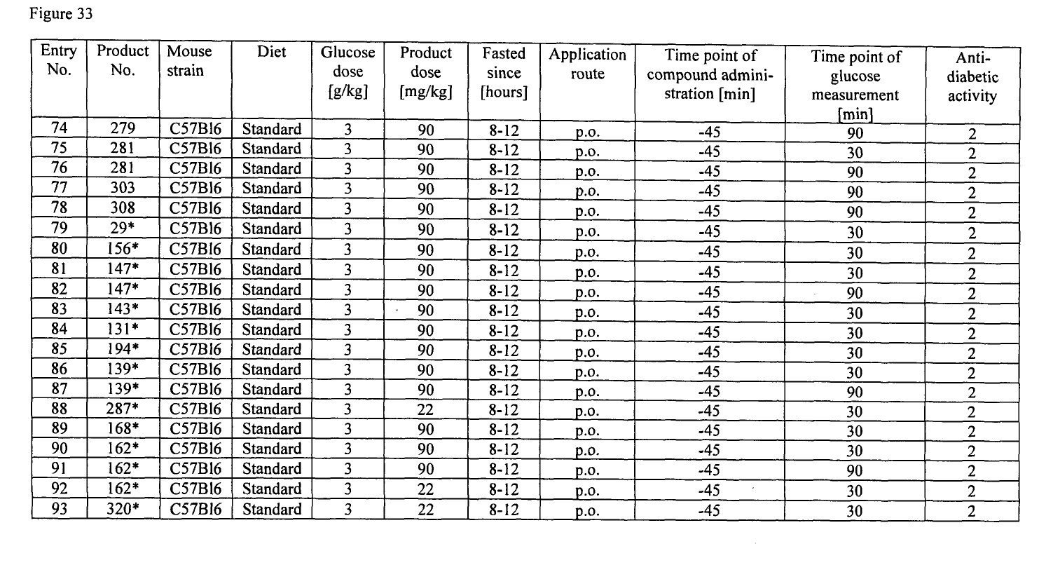

All animal experiments described below and listed in Table 2 (FIGS. 30 to 33) were performed in accordance to Austrian law and the principles of good laboratory animal care. Data shown in Table 2 are obtained using commercially available male mice purchased, e.g. from the breeding facilities of the Medical University of Vienna (Austria) or Charles River Lab. (USA). Male mice were used at the age of 7-30 weeks and had free access to food and water except for defined fasting periods before experimentation. They were maintained at room temperature and a 12 h/12 h light-dark cycle. Mice were fasted for 8-12 hours prior to oral glucose tolerance testing.

The antidiabetic activities of the products of formula I or II were evaluated in oral glucose tolerance tests in mice, in analogy to the procedure known to the general physician. Each mouse was either treated per os via gavage or injected intraperitoneally with a product of formula I or II as specified in Table 2. A control group treated with vehicle containing no product of formula I or II was examined in parallel in each test run. This was followed by oral administration via gavage of a glucose solution (1-3 g/kg as specified) at T=0 min. Blood was collected via puncture of the tip of the tail immediately before administration of products of formula I or II, immediately before administration of glucose, and at T=30 min and/or T=90 and/or T=150 as the case may be. Blood glucose was determined using portable glucometers as commonly used in human diabetes.

The increment in blood glucose at T=min over levels measured at T=0 min was calculated for each animal. Mean values of the increment for treatment group and vehicle group were compared (typical group size n=6-10 mice). Percent reduction induced by products of formula I or II versus vehicle was the readout parameter for glucose-lowering activity. As listed in Table 2, an effect of 1 means a reduction of 15-30% of incremental blood glucose at the given time point T=min versus vehicle group, an effect of 2 means a reduction of more than 30% of incremental blood glucose at the given time point T=min versus vehicle group. The statistical significance of differences was assessed by standard methodologies, for example 2-tailed unpaired Student's T test; p<0.05 was considered statistically significant.

For biological testing, products of formula I or II were dissolved or suspended in 0.5% carboxymethylcellulosis containing 1-2% acetic acid. The vehicle group received the same amount of a 0.5% carboxymethylcellulosis solution containing 1-2% acetic acid.

Mice were permanently held on a standard laboratory chow diet (kg/kg: <10% crude fat) or on a high fat diet 1 (HFD1; energy content: 72% fat, <1% carbohydrate) as specified. Experiments with mice on a high fat diet were performed after a pre-feeding period of 5-6 weeks on HFD1.

Antidiabetic effects of products are listed in Table 2 as evaluated in a glucose tolerance test in mice.

1. A compound having formula I: 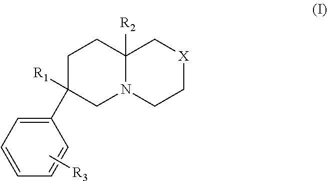
wherein:
R¹=C₁-C₆ alkyl, Phenyl, or substituted Phenyl;
R²=H, C₁-C₆ alkyl, or alkyl-cycloalkyl;
R³=(R³¹)ₖ wherein k=0, 1, 2, or 3 and R³¹=H, F, Cl, Br, CF₃, or C₁-C₆ alkyl; or
(R³²)ₖ wherein k=4, or 5 and R³²=H, or F;
X=Carbonyl, R⁹, CR⁴CN, CHR⁵, CH(COH(CH₃)₂), CR⁴(OR⁶), CR⁶(OR⁴), CR⁶benzyloxy, CR⁶(2-methoxyethoxy), CR⁶[(2-methoxyethoxy)methoxy], CR⁶[(2-methoxyethoxy)ethoxy], CR⁴(CO)OR⁴, CR⁴(CO)N(R⁴)₂, CR⁴(CO)R⁵, CR⁴(CO)R⁴ CR⁴(CH₂)ₖ(Y)ₘ(CH₂)ₙZ, C(OR⁴)(CH₂)ₖ(Y)ₘ(CH₂)ₙZ, or C(Otrimethylsilyl)(CH₂)ₖ(Y)ₘ(CH₂)ₙZ;
wherein k=1, 2, 3, or 4; m=0, or 1; n=0, 1, 2, or 3;
Y=CR⁴, R⁶, 1,1-cyclopentyl, or 1,1-cyclohexyl;
Z=R⁵, R⁶, R⁷, R⁸, CN, (CO)OR⁶, (CO)R⁴, OR⁶, OR⁷, O(CO)R⁵, (CO)R⁵, (CO)R⁸, O(CH₂)_{2 or 3}R⁵, O(CH₂)_{2 or 3}R⁶, O(CH₂)_{2 or 3}R⁷, O(CH₂)_{2 or 3}R⁸, O(CH₂)_{2 or 3}OR⁶, O(CH₂)_{2 or 3}OR⁷, NR⁴(CO)OR⁶, NR⁴(CO)R⁵, NR⁴(CH₂)_{2 or 3}R, NR⁴(CH₂)_{2 or 3}R⁶, NR⁴(CH₂)_{2 or 3}R⁷, NR⁴(CH₂)_{2 or 3}R⁸, NR⁴(CH₂)_{2 or 3}OR⁶, or NR⁴(CH₂)_{2 or 3}OR⁷;
wherein:
R⁴=H, or C₁-C₆ alkyl; 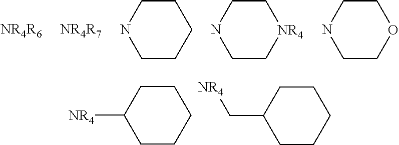
R⁵=one of: 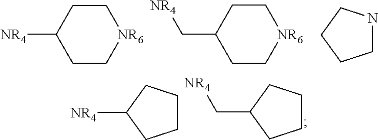
R⁶=H, C₁-C₆ alkyl, isopropyl, isobutyl, secbutyl, t-Butyl, cyclopentyl, cyclohexyl, cycloheptyl, cyclopentylmethylen, or cyclohexylmethylen;
R⁷=phenyl, monofluorophenyl, difluorophenyl, trifluorophenyl, trifluoromethylphenyl, chlorophenyl, dichlorophenyl, monofluoro-monochlorophenyl, difluoro-monochlorophenyl, monofluoro-monomethylphenyl, methylphenyl, or dimethylphenyl;
R⁸=one of: 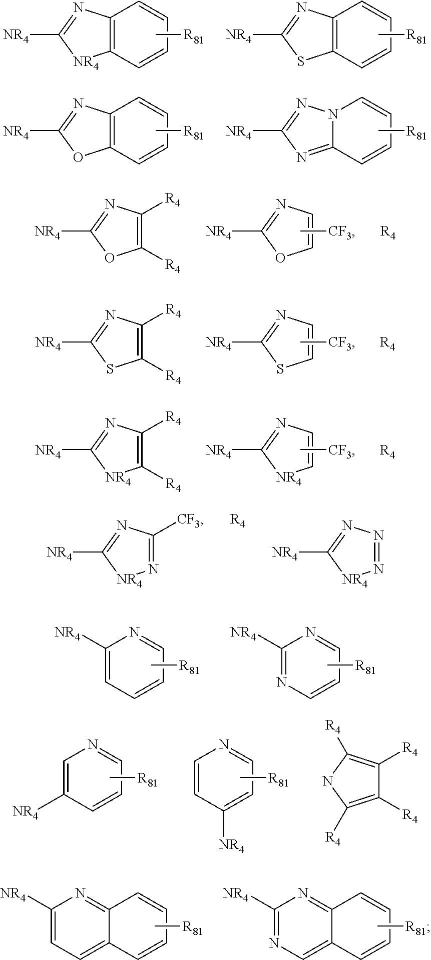
R⁸¹=(F, Cl, CF₃, or C₁-C₆ alkyl)_{0, 1 or 2} independently of each other;
R⁹=one of:

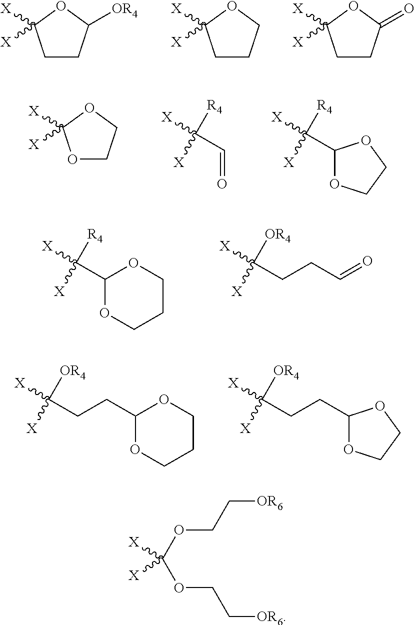

2. The compound according to claim 1 having formula II: 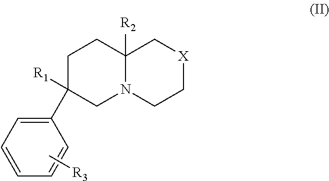
wherein:
R¹=Methyl, or Phenyl;
R²=H, or Methyl;
R³=H, F, Cl, CF₃, difluoro, trifluoro, dichloro, monofluoro-monochloro, methyl, dimethyl, or monofluoro-monomethyl;
X=Carbonyl, R⁹, CR⁴CN, CHR⁵, CH(COH(CH₃)₂), CR⁴(OR⁶), CR⁶(OR⁴), CR⁶benzyloxy, CR⁶(2-methoxyethoxy), CR⁶[(2-methoxyethoxy)methoxy], CR⁶[(2-methoxyethoxy)ethoxy], CR⁴(CO)OR⁴, CR⁴(CO)N(R⁴)₂, CR⁴(CO)R⁵, CR⁴(CO)R⁴, CR⁴(CH₂)ₖ(Y)ₘ(CH₂)ₙZ, C(OR⁴)(CH₂)ₖ(Y)ₘ(CH₂)ₙZ, or C(Otrimethylsilyl)(CH₂)ₖ(Y)ₘ(CH₂)ₙZ;
wherein k=1, 2, or 3; m=0, or 1; n=0, 1, or 2;
Y=CR⁴, R⁶, 1,1-cyclopentyl, or 1,1-cyclohexyl;
Z=R⁵, R⁶, R⁷, R⁸, CN, (CO)OR⁶, (CO)R⁴, OR⁶, (CO)R⁵, (CO)R⁸, or NR⁴(CO)R⁵;
wherein:
R⁴=H, or C₁-C₆ alkyl;
R⁵=one of: 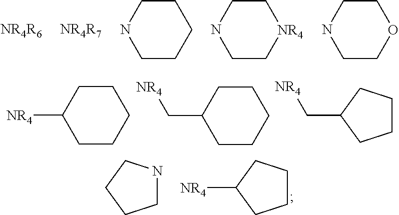
R⁶=H, C₁-C₆ alkyl, isopropyl, isobutyl, secbutyl, t-Butyl, cyclopentyl, or cyclohexyl;
R⁷=phenyl, monofluorophenyl, difluorophenyl, trifluorophenyl, trifluoromethylphenyl, chlorophenyl, dichlorophenyl, monofluoro-monochlorophenyl, difluoro-monochlorophenyl, monofluoro-monomethylphenyl, methylphenyl, or dimethylphenyl;
R⁸=one of: 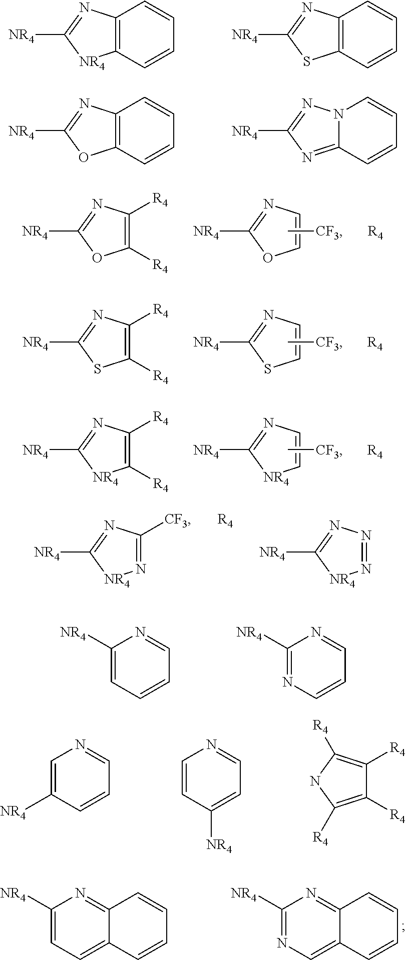
R⁹=one of:

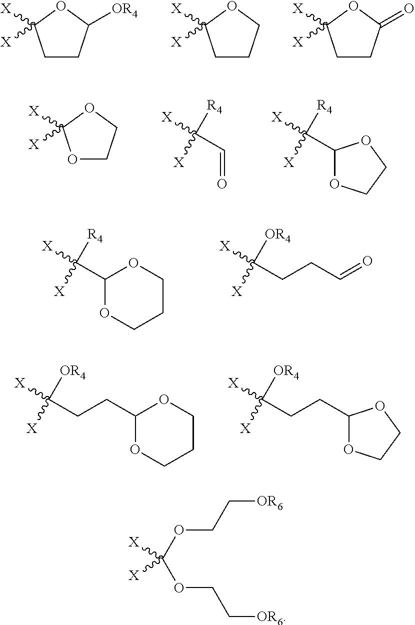

3. A compound having a structure of one of the following product numbers (PN): 29, 79, 83, 111, 131, 135, 139, 143, 147, 156, 158, 160, 162, 166, 168, 190, 194, 224, 230, 249, 287, or 320:

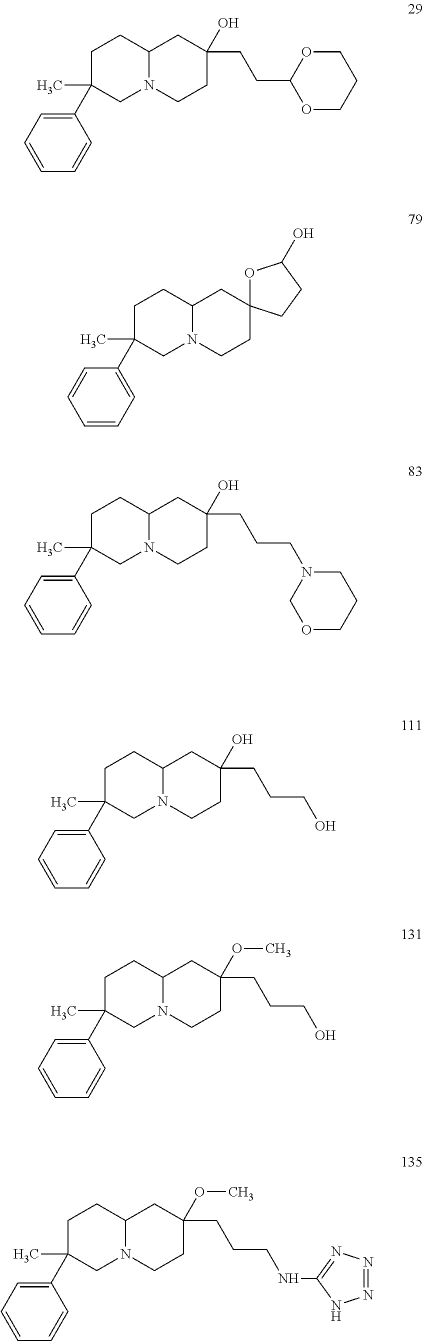

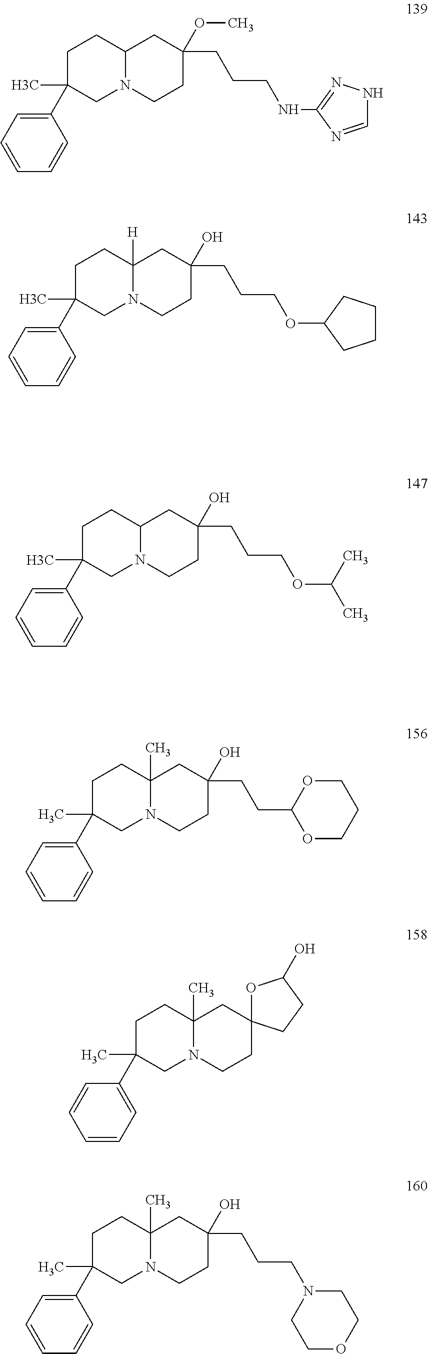

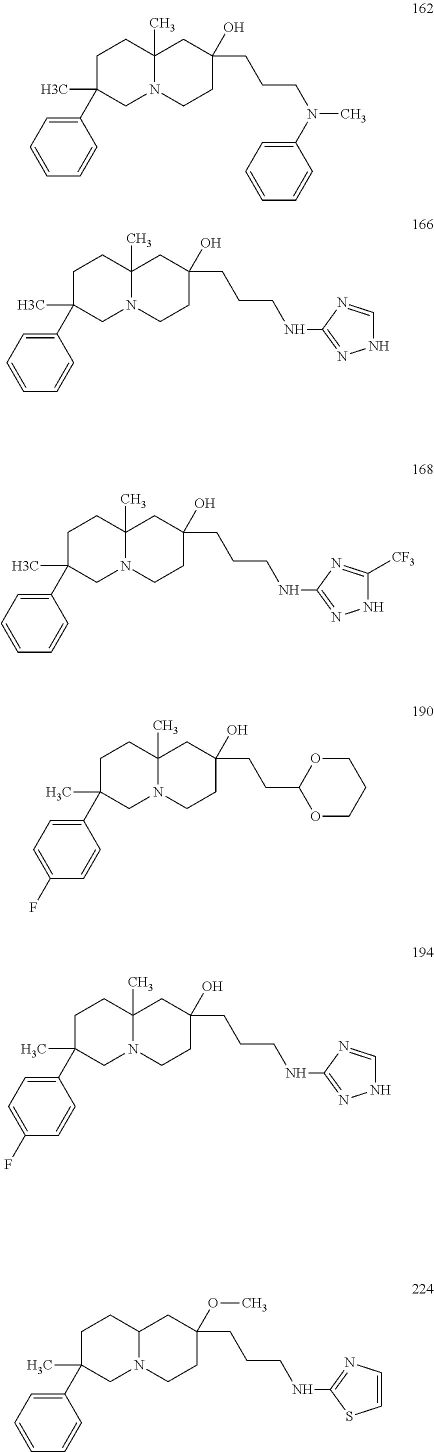

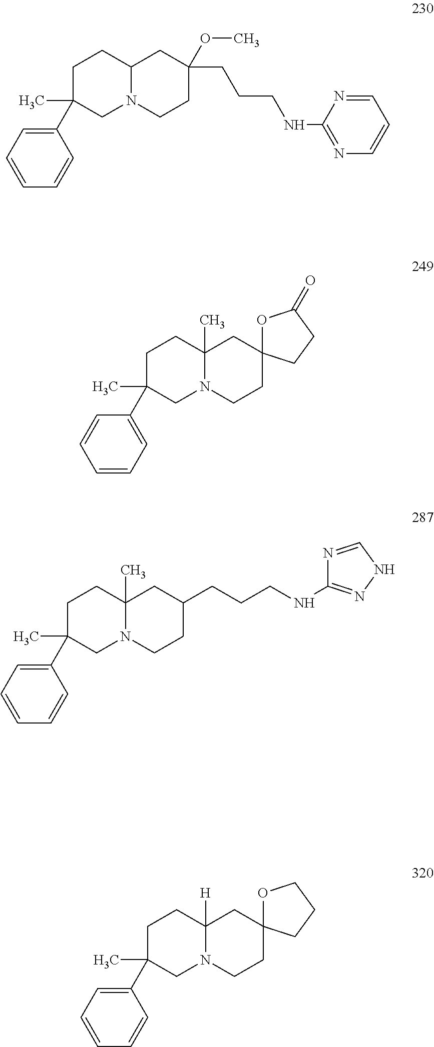

4. A pharmaceutical composition containing a compound of formula I of claim 1 as a drug substance together with one or more pharmaceutically acceptable carriers.

5.-10. (canceled)

11. A pharmaceutical composition containing a compound of formula II of claim 2 as drug substance together with one or more pharmaceutically acceptable carriers.

12. A pharmaceutical composition containing a compound of claim 3 as a drug substance together with one or more pharmaceutically acceptable carriers.

13. A method of treatment or prevention of diabetes mellitus, hyperlipidemia, diabetic dyslipidemia, metabolic syndrome, obesity, or diseases related to metabolic dysfunction comprising administering to a patient a therapeutically effective amount of a compound of formula I as defined in claim 1.

14. A method of treatment or prevention of diabetes mellitus, hyperlipidemia, diabetic dyslipidemia, metabolic syndrome, obesity, or diseases related to metabolic dysfunction comprising administering to a patient a therapeutically effective amount of a compound of formula II as defined in claim 2.

15. A method of treatment or prevention of diabetes mellitus, hyperlipidemia, diabetic dyslipidemia, metabolic syndrome, obesity, or diseases related to metabolic dysfunction comprising administering to a patient a therapeutically effective amount of a compound of claim 3.
